SEQUENCE LISTING (18 sequences)

SEQ ID NO: 1 - DNA, 1878 bp, Homo sapiens
gcaggaattcggcacgagctccagcccgcccagtggccgcagtcacccaggtccagaggcggcggtatcacaggctctccgacatgtctatgctggctgaacgtcggcggaagcagaagtgggctgtggatcctcagaacactgcctggagtaatgacgattccaagtttggccagcggatgctagagaagatggggtggtctaaaggaaagggtttaggggctcaggagcaaggagccacagatcatattaaagttcaagtgaaaaataaccacctgggactcggagctaccatcaataatgaagacaactggattgcccatcaggatgattttaaccagcttctggccgaactgaacacttgccatgggcaggaaaccacagattcctcggacaagaaggaaaagaaatcttttagccttgaggaaaagtccaaaatctccaaaaaccgtgttcactatatgaaattcacaaaagggaaggatctgtcatctcggagcaaaacagatcttgactgcatttttgggaaaagacagagtaagaagactcccgagggcgatgccagtccctccactccagaggagaacgaaaccacgacaaccagcgccttcaccatccaggagtactttgccaagccggtggcagcactgaagaacaagccccaggttccagttccagggtctgacatttctgagacgcaggtggaacgtaaaagggggaagaaaagaaataaagaggccacaggtaaagatgtggaaagttacctccagcctaaggccaagaggcacacggagggaaagcccgagagggccgaggcccaggagcgagtggccaagaagaagtgcgcgccagcagaaaaacagctcagaggcccctgctgggaccagagttccaaggcctctgctcaggatgcaggggaccatgtgcagccgcctgagggccgggacttcaccctgaagcccaaaaagaggagagggaagaaaaagctgcaaaaaccagtagagatagcagaggacgctacactagaagaaacgctagtgaaaaagaagaagaagaaagattccaaatgaatccttcccagccggggccttccgaccactcagctgtcagggcactgcgggggcagacacctctggcctgaagtcacagcagagttcaccccagagcgcctgggcgcatcttgtggcatgcccatgggctgccgagtcctgccctctcgccacatttcccccaagttacattcccaggaggacctttttaatgttctcaatcgtggctctcagacacaaataaattctcgtgccgaattcggcacgagctcgctctcattcctgatgtggacatcgactccgacggcgtcttcaagtatgtgttgatccgagtccactcggctccccgctccggggctccggctgcagagagcaaggagatcgtgcgcggctacaagtgggctgagtaccatgcggacatctacgacaaagtgtcgggcgacatgcagaagcaaggctgcgactgtgagtgtctgggcggcgggcgcatctcccaccagagtcaggacaagaagattcacgtgtacggctattccatggcctatggtcctgcccagcacgccatttcaactgagaaaatcaaagccaagtaccccgactacgaggtcacctgggctaacgacggctactgagcactcccagcccggggcctgctgcctccagcagccacttcagagcccccgcctttgcctgcactcctcttgcagggctggccctgcctgctcctgcggcagcctctggtgacgtgctgtccaccaggccttggagacaggctagcctggccacagaattaaacgtgttgccacaccaaaaaaaaaaaaaaaaaaaa

SEQ ID NO: 2 - DNA, 1036 bp, Homo sapiens
tgcgcgccagcagaaaaacagctcagaggcccctgctgggaccagagttccaaggcctctgctcaggatgcaggggaccatgtgcagccgcctgagggccgggacttcaccctgaagcccaaaaagaggagagggaagaaaaagctgcaaaaaccagtagagatagcagaggacgctacactagaagaaacgctagtgaaaaagaagaagaagaaagattccaaatgaatccttcccagccggggccttccgaccactcagctgtcagggcactgcgggggcagacacctctggcctgaagtcacagcagagttcaccccagagcgcctgggcgcatcttgtggcatgcccatgggctgccgagtcctgccctctcgccacatttcccccaagttacattcccaggaggacctttttaatgttctcaatcgtggctctcagacacaaataaattctcgtgccgaattcggcacgagctcgctctcattcctgatgtggacatcgactccgacggcgtcttcaagtatgtgttgatccgagtccactcggctccccgctccggggctccggctgcagagagcaaggagatcgtgcgcggctacaagtgggctgagtaccatgcggacatctacgacaaagtgtcgggcgacatgcagaagcaaggctgcgactgtgagtgtctgggcggcgggcgcatctcccaccagagtcaggacaagaagattcacgtgtacggctattccatggcctatggtcctgcccagcacgccatttcaactgagaaaatcaaagccaagtaccccgactacgaggtcacctgggctaacgacggctactgagcactcccagcccggggcctgctgcctccagcagccacttcagagcccccgcctttgcctgcactcctcttgcagggctggccctgcctgctcctgcggcagcctctggtgacgtgctgtccaccaggccttggagacaggctagcctggccacagaattaaacgtgttgccacaccaaaaaaaaaaaaaaaaaaaa

SEQ ID NO: 3 - protein, 328 aa, Homo sapiens [sequence omitted]

SEQ ID NO: 4 - protein, 75 aa, Homo sapiens
CAPAEKQLRGPCWDQSSKASAQDAGDHVQPPEGRDFTLKPKKRRGKKKLQKPVEIAEDATLEETLVKKKKKKDSK

SEQ ID NO: 5 - DNA, 1236 bp, Homo sapiens
atgttgatgctggctgagcagcagcagaagcagaagtgggctgtgaatactcaaaacactgcctggagtaatgctgattctaaatttggccagaggatactagagaagatggaatggtctaaaggaaggggtttaggggttcaggagcaaggaggcccagatgatattaaagttcaagttaaaaataacgacctgggacttcaagctacaatcaataatgaagccaactggattgcccatcaagatgattttaactggcttctggcggaactgaacacttgtcagaggcaggaaacagcagactccttagacaacaagaaaaagaaatattttagtcttgaagaaattccaaaatcttcaaaaaactgtgttcatcataggaaatttacaaaagaaaaggatctatcatctcggagcaaaacagatcgtgactgcatttttgggaaaaaacagagtaagaagactcccgagggtaattccagtccctccactccagacaagaacaaaaccacgatgacaacccatgccttcaccatccaggagcgttttgccaagcgaatggcagcactgaagaacaagccccaggttgcagctccagggcctgacatttccaagacccaagtggaatgcaaaagggggaagaaaagaaacaaagaggcaacaggtaaaaatggggagagttaccccccaacacagcctaaggccaagcggcctaaagagggaaagcctaagagagacaaggtccagaagtcggcatccaaggagaaaagagcacggacagacggacagtgcagaggcctctgctgggaagagagttctgaggcctctgctcagggtgcagggaattgtgtgcagccacctgatggccaggatttcaccctgaagcccaaaaagacaagaggaaaaaaaaaagctgcaaagccagtagaggtagcaatggacactacgctgaaagaaacaccaatgaaaaataagaaaaagaagaaaggttccaaatgaattctctccagccagggccttccgaccactcagcttgtcagggcgctgctggggcagacacctctggcctgaagtcagagcagagttcaccccagagagccggggcacatcttgtgacatgcctgtgggtggccgagtctcgccctctcaccacatttctccccaagttatgttcccaggagggctttttttaaatgttctaaatcatggctttcataaacaaatacatttttgtaa

SEQ ID NO: 6 - protein, 332 aa, Homo sapiens
MLMLAEQQQKQKWAVNTQNTAWSNADSKFGQRILEKMEWSKGRGLGVQEQGGPDDIKVQVKNNDLGLQATINNEANWIAHQDDFNWLLAELNTCQRQETADSLDNKKKKYFSLEEIPKSSKNCVHHRKFTKEKDLSSRSKTDRDCIFGKKQSKKTPEGNSSPSTPDKNKTTMTTHAFTIQERFAKRMAALKNKPQVAAPGPDISKTQVECKRGKKRNKEATGKNGESYPPTQPKAKRPKEGKPKRDKVQKSASKEKRARTDGQCRGLCWEESSEASAQGAGNCVQPPDGQDFTLKPKKTRGKKKAAKPVEVAMDTTLKETPMKNKKKKKGSK

SEQ ID NO: 7 - protein, 271 aa, Saccharomyces cerevisiae
MGLAATRTKQRFGLDPRNTAWSNDTSRFGHQFLEKFGWKPGMGLGLSPMNSNTSHIKVSIKDDNVGLGAKLKRKDKKDEFDNGECAGLDVFQRILGRLNGKESKISEELDTQRKQKIIDGKWGIHFVKGEVLASTWDPKTHKLRNYSHAKNRKREGDDSEDEDDADQEDKDSDKKKHKKHKKHKKDKKKDKKAKKEHKKHKKEEKRLKKEKRAEKTKETKKTSKLKSSESASNIPDAVNTRLSVRSKWIKQKRAALMDSKALNEIFMITND

SEQ ID NO: 8 - protein, 339 aa, Caenorhabditis elegans
MSILAEPKRKQKISIDPQNLTWKNDDQKLSKKLMEKMGWSEGDGLGRNRQGNADSVKLKANTSGRGLGADKMADYDSTWISHHDDFADLLAALNKNKEQEPEQTEEEKNAAAEKISIELKSKSIRRRIHYQKFTRAKDTSNYSDSHKKGILGYGRLKSDNAEEKIEEKTENSSVKSDSSDSQADSQEKKEGNNTVSTLSVGDYFAAKMAALKAKREAAAANQTEVKMEIKTEVEEEESDEEKARRKAEKKERKRLRREQRDKEETLETVETILEVKQEVKEEIIDEEFDEAERKRLKKEKKRLKRLREQQQPENEGAEGGEADEEEIPRKRKKHTEDEH

SEQ ID NO: 9 - DNA, 26 bp, Artificial Sequence
  Synthetic primer
ttagggttagggttaggggggggggg

SEQ ID NO: 10 - DNA, 26 bp, Artificial Sequence
  Synthetic sequence
ttagggttagggttgggggggggggg

SEQ ID NO: 11 - DNA, 26 bp, Artificial Sequence
  Synthetic Primer
ttagggttagggtggggggggggggg

SEQ ID NO: 12 - DNA, 16 bp, Artificial Sequence
  Synthetic Sequence
cccccccctaacccta

SEQ ID NO: 13 - DNA, 16 bp, Artificial Sequence
  Synthetic primer
ccccccccaaccctaa

SEQ ID NO: 14 - DNA, 16 bp, Artificial Sequence
  Synthetic primer
ccccccccaccctaac

SEQ ID NO: 15 - DNA, 6 bp, Artificial Sequence
  Synthetic sequence
ttaggg

SEQ ID NO: 16 - DNA, 6 bp, Artificial Sequence
  Synthetic probe
ttaggg

SEQ ID NO: 17 - protein, 419 aa, Homo sapiens
MAEDVSSAAPSPRRCADGRDADPTEEQMAETERNDEEQFECQELLECQVQVGAPEEEEEEEEDAGLVAEAEAVAAGWMLDFLCLSLCRAFRDGRSEDFRRTRNSAEAIIHGLSSLTACQLRTIYICQFLTRIAAGKTLDAQFENDERITPLESALMIWGSIEKEHDKLHEEIQNLIKIQAIAVCMENGNFKEAEEVFERIFGDPNSHMPFKSKLLMIISQKDTFHSFFQHFSYNHMMEKIKSYVNYVLSEKSSTFLMKAAAKVVESKRTRTITSQDKPSGNDVEMETEANLDTRKRSHKNLFLSKLQHGTQQQDLNKKERRVGTPQSTKKKKESRRATESRIPVSKSQPVTPEKHRARKRQAWLWEEDKNLRSGVRKYGEGNWSKILLHYKFNNRTSVMLKDRWRTMKKLKLISSDSED

SEQ ID NO: 18 - DNA, 1929 bp, Homo sapiens
atggcggaggatgtttcctcagcggccccgagcccgcggcggtgtgcggatggtagggatgccgaccctactgaggagcagatggcagaaacagagagaaacgacgaggagcagttcgaatgccaggaactgctcgagtgccaggtgcaggtgggggcccccgaggaggaggaggaggaggaggaggacgcgggcctggtggccgaggccgaggccgtggctgccggctggatgctcgatttcctctgcctctctctttgccgagctttccgcgacggccgctccgaggacttccgcaggacccgcaacagcgcagaggctattattcatggactatccagtctaacagcttgccagttgagaacgatatacatatgtcagtttttgacaagaattgcagcaggaaaaacccttgatgcacagtttgaaaatgatgaacgaattacacccttggaatcagccctgatgatttggggttcaattgaaaaggaacatgacaaacttcatgaagaaatacagaatttaattaaaattcaggctatagctgtttgtatggaaaatggcaactttaaagaagcagaagaagtctttgaaagaatatttggtgatccaaattctcatatgcctttcaaaagcaaattgcttatgataatctctcagaaagatacatttcattccttttttcaacacttcagctacaaccacatgatggagaaaattaagagttatgtgaattatgtgctaagtgaaaaatcatcaacctttctaatgaaggcagcggcaaaagtagtagaaagcaaaaggacaagaacaataacttctcaagataaacctagtggtaatgatgttgaaatggaaactgaagctaatttggatacaagaaaaaggtctcacaagaatcttttcttatctaagttgcaacatggaacccagcaacaagaccttaataagaaagaaagaagagtaggaactcctcaaagtacaaaaaagaaaaaagaaagcagaagagccactgaaagcagaatacctgtttcaaagagtcagccggtaactcctgaaaaacatcgagctagaaaaagacaggcatggctttgggaagaagacaagaatttgagatctggcgtgaggaaatatggagagggaaactggtctaaaatactgttgcattataaattcaacaaccggacaagtgtcatgttaaaagacagatggaggaccatgaagaaactaaaactgatttcctcagacagcgaagactgattgtgtttgtaaaagcttgatgaaaggacagttaagtattttgatcactgcattttgtttgaaacttgtgtcattgatgtaatttaaaacttttgtttaaagcattacagtatttttctgtgaccatcaattaatgagggtttgtgctaccagagttaaagcatatgctatcattgtattctttaagaaccttattttgataaaatgtaaatttgttgaaccctgccacatttagtatccccacccccaaatcctgttccaatgaaaaaattaaaacctgatacgaaaaaaaaaaaattccagttaacctattttgtgtctgtaggctgacctcaaccctgtaacgtaacccattaaaatgaatttctttttttttaagacagagtttctctctgttgcccaggctggagtgcagtggcgcaatttcagctcactgaacctctgcctcccaggtcaagtgattctcctgcctcagcctctgagtagctgggattacaggcacacaccaccagccagctaatttttgtatttttagtagaggcggggtttcaccatgctggtcaggatgttctccaactcctgacttcatgatccacccacctcggcctcccaaagtgctgagattacagacgtgagccactgcgtcctgcctaaaatgaattttctaga

(19) United States
(12) Patent Application Publication
(10) Pub. No.: US 2004/0142357 A1
(43) Pub. Date: Jul. 22, 2004

(54) Novel telomerase inhibitors and uses therefor

(75) Inventors: Kun Ping Lu, Newton, MA (US); Xiao Zhen Zhou, Newton, MA (US)

Correspondence Address: PALMER & DODGE, LLP, KATHLEEN M. WILLIAMS, 111 HUNTINGTON AVENUE, BOSTON, MA, 02199, US

(73) Assignee: Beth Israel Deaconess Medical Center

(21) Appl. No.: 10/705,531

(22) Filed: Nov. 10, 2003

Related U.S. Application Data
(63) Continuation of application No. PCT/US02/14927, filed on May 10, 2002.
(60) Provisional application No. 60/290,363, filed on May 11, 2001.

(51) Int. Cl. C12Q 1/68; C07H 21/04; C12N 9/99

(52) U.S. Cl. 435/6; 435/69.2; 435/184; 435/320.1; 435/325; 530/388.26; 536/23.2

(57) ABSTRACT

The present invention provides molecules including Pin2 and telomerase binding polypeptides, polynucleotides encoding such polypeptides, and antibodies specifically immunoreactive to said polypeptides. The invention also provides methods for screening agents which modulate the function or expression of said Pin2 and telomerase binding polypeptides. Methods are provided for disease diagnosis and treatment using said agents, said antibodies and oligonucleotides derived from the above polynucleotides.

### RELATED APPLICATIONS

This application is a continuation of International Application No. PCT/US02/14927 with an International Filing date of May 10, 2002, which claims priority to U.S. provisional application no. 60/290,363 filed on May 11, 2001.

### GOVERNMENT INTERESTS

This invention was made with government support under grant numbers R01 GM56230 and R01 GM58556, awarded by the National Institutes of Health. The government has certain rights in the invention.

### FIELD OF INVENTION

The invention is related to novel polynucleotides encoding polypeptides comprising telomerase inhibiting activities, as well as methods for cancer diagnosis, cancer treatment and aging prevention using said polynucleotides and polypeptides.

### BACKGROUND

Telomere and Telomerase

Telomerase is a ribonucleoprotein enzyme that synthesizes one strand of the telomeric DNA using as a template a sequence contained within the RNA component of the enzyme. The ends of chromosomes have specialized sequences, termed telomeres, comprising tandem repeats of simple DNA sequences which in humans is 5′-TTAGGG (SEQ ID No. 15, see Blackburn, 1991). Apart from protecting ends of chromosomes telomeres have several other functions, the most important of which appear to be associated with replication, regulating the cell cycle clock and ageing (Counter et al., 1992). Progressive rounds of cell division shorten telomeres by 50-200 nucleotides per round. Almost all cancer cells have shortened telomeres, which are maintained at a constant length (Allshire et al., 1988; Harley et al., 1990; Harley et al., 1994) and are associated with chromosome instability and cell immortalization.

With regard to human cells and tissues telomerase activity has been identified in immortal cell lines and in most tumors (Kim et al., 1994) but has not been detected at biologically significant levels (that are required to maintain telomere length over many cell divisions) in mortal cell strains or in normal non-germline tissues (Counter et al., 1992; Counter et al, 1994). These observations suggest telomerase activity is directly involved in telomere maintenance, linking this enzyme to cell immortality.

As described above, the immortalization of cells involves the activation of telomerase. More specifically, the connection between telomerase activity and the ability of many cancer cell lines, including skin, connective tissue, adipose, breast, lung, stomach, pancreas, ovary, cervix, uterus, kidney, bladder, colon, prostate, central nervous system (CNS), retina and blood cancer cell lines, to remain immortal has been demonstrated by analysis of telomerase activity (Kim, et al., 1994). This analysis, supplemented by data that indicates that the shortening of telomere length can provide the signal for replicative senescence in normal cells, see PCT Application No. 93/23572, incorporated herein by reference, demonstrates that inhibition of telomerase activity can be an effective ant-icancer therapy. Thus, telomerase activity can prevent the onset of otherwise normal replicative senescence by preventing the normal reduction of telomere length and the concurrent cessation of cell replication that occurs in normal somatic cells after many cell divisions. In cancer cells, where the malignant phenotype is due to loss of cell cycle or growth controls or other genetic damage, an absence of telomerase activity permits the loss of telomeric DNA during cell division, resulting i;n chromosomal rearrangements and aberrations that lead ultimately to cell death. However, in cancer cells having telomerase activity, telomeric DNA is not lost during cell division, thereby allowing the cancer cells to become immortal, leading to a terminal prognosis for the patient.

Many physiological changes occur as humans age. In addition to those observed at the phenotypic level such as change in hair color, appearance of skin, decreased lean body mass, etc., there are many changes at the cellular and biochemical levels. One such change that has been observed is a marked decrease in the length of telomeres in somatic cells as they age (Harley et al., 1990 nature, 345:458-460). Telomeres are repetitive DNA sequences that are localized to the ends of every chromosome, and are necessary for proper chromosome maintenance, replication, and localization of the chromosomes within the cell nucleus.

In most organisms, telomeres are synthesized and maintained by an enzyme known as telomerase. Telomerase is a ribonucleoprotein composed of RNA and protein components, and both types of components are necessary for activity (see for example, Greider, 1996 Annu. Rev. Biochem., 65:337-365; Greider et al., 1996 in Cellular Aging and Cell Death, Wiley-Liss Inc., New York, N.Y., pp. 123-138).

Most cells of adult humans do not have telomerase activity; exceptions include, for example, germline tissues (sperm cells and oocytes) and certain blood cells (Greider et al., Cellular Aging and Cell Death, supra). Telomeres have several functions apart from protecting the ends of chromosomes, the most important of which appear to be associated with senescence, replication, and the cell cycle clock (Counter et al., 1992). Progressive rounds of cell division result in a shortening of the telomeres by some 50-200 nucleotides per round. Almost all cancer cells have telomeres, which are maintained at a constant length (Allshire et al., 1988; Harley et al., 1990; Harley et al., 1994) and are associated with chromosome instability and cell immortalization.

Decreased telomere length correlates well with decreased replicative capacity of cells in culture (referred to as cellular senescence or cell age). It has been postulated that shortened telomeres may be involved in the inability of cells to continue dividing (Harley, supra; Levy et al., 1992 J. Mol. Biol., 225:951-960; and Harley et al., 1994 Cold Spring Harbor Symposium on Quantitative Biology, 59:307-315), thereby contributing to senescence of the cells.

The enzyme telomerase adds the telomeric repeat sequences onto telomere ends, ensuring the net maintenance of telomere length in cancer cells commensurate with successive roundus of cell division. A significant recent finding has been that approximately 85-90% of all human cancers are positive for telomerase, both in cultured cancer cells and primary cancer tissue, whereas most somatic cells appear to lack detectable levels of telomerase (Kim et al, 1994; Hiyama et al., 1995a). This finding has been extended to a wide range of human cancers (see, for example, references Broccoli, 1994 and Hiyama et al., 1995b) and is likely to be of use in diagnosis.

Human telomerase has since been proposed as a novel and potentially highly selective target for anticancer drug design (Feng et al., 1995; Rhyu et al., 1995; Parkinson, 1996).

Chemical agents have been reported as DNA-interactive agents which inhibits telomerase activity (Collier and Neidle, 1988; 1992; Agbandje et al., 1992). These compounds have been shown to act as selective DNA triplex interactive compounds, with reduced affinity for duplex DNA and only moderate conventional cytotoxicity in a range of cancer cell lines. US Pat. Nos. 5,863,936; 5,770,613; 5,767,278; 5,760,062; 5,703,116; 5,656,638; 6,087,493; 6,156,763 also describe chemical telomerase inhibitors for treating cancer and other diseases.

Studies with antisense constructs against telomerase RNA in HeLa cells show that telomere shortening is produced, together with the death of these otherwise immortal cells (Feng et al., 1995). Sequence-specific peptide-nucleic acids directed against telomerase RNA have also been found to exert an inhibitory effect on the enzyme (Norton et al., 1996; U.S. Pat. No. 6,194,206). Oligonucleotides have been designed to bind to a telomere to block the ability of telomerase to bind to that telomere (U.S. Pat. No. 6,194,206).

Despite the above discovery of telomerase inhibitors described above, there remains a need for identify naturally occurring molecules that act as telomerase inhibitors and for novel compositions and methods for treating cancer and other diseases related to telomerase activity. The present invention meets these and other needs.

Pin2 Protein

Telomeres are essential for preserving chromosome integrity during the cell cycle and have been specifically implicated in mitotic progression, but little is known about the signaling molecule(s) involved. The human telomeric repeat binding factor protein (TRF1) is shown to be important in regulating telomere length (Chong, L, Van Steensel, B., Broccoli, D., Erdjument, B. H., Hanish, J., Tempst, P. & de Lange, T. (1995) Science 270, 1663-1667; Bilaud, T., Koering, C. E., Binet, B. E., Ancelin, K., Pollice, A., Gasser, S. M. & Gilson, E. (1996) Nucleic Acids Res. 24, 1294-1303). However, nothing is known about its function and regulation during the cell cycle.

Pin2 protein is identical in sequence to TRF1 apart from an internal deletion of 20 amino acids; Pin2 and TRF1 may be derived from the same gene, Pin2/TRF1 (Shen et al., 1997). The crystal structure of the yeast telomeric protein Raplp reveals that both its HTH domains interact with the telomeric DNA (Konig, P., Giraldo, R., Chapman, L & Rhodes, D. (1996) Cell 85, 125-136). In contrast, Pin2 and TRF1 contain only a single HTH domain.

Shen et al. demonstrated Pin2 to be the major expressed product and to form homo- and heterodimers with TRF1; both dimers were localized at telomeres (Shen et al., 1997, Characterization and cell cycle regulation of the related human telomeric proteins Pin2 and TRF1 suggest a role in mitosis, Proc. Natl. Acad. Sci. USA, 94: 13618-13623, hereby incorporated by reference).

Pin2 directly binds the human telomeric repeat DNA in vitro, and is localized to all telomeres uniformly in telomerase-positive cells. In contrast, in cell lines that contain barely detectable telomerase activity, Pin2 is highly concentrated at only a few telomeres.

The protein level of Pin2 is highly regulated during the cell cycle, being strikingly increased in G2+M and decreased in G1 cells; overexpression of Pin2 results in an accumulation of HeLa cells in G2+M (Shen et al., supra).

These results indicate that Pin2 is the major human telomeric protein and is highly regulated during the cell cycle, with a possible role in mitosis. The results also suggest that Pin2/TRF1 may connect mitotic control to the telomere regulatory machinery whose deregulation has been implicated in cancer and aging.

It is an object of this invention to provide novel polynucleotides and polypeptides that modulate telomerase activity.

It is a further object to provide methods of altering the function or expression of such nucleic polynucleotides and polypeptides in the human body for the treatment of telomerase activity related diseases.

### BRIEF DESCRIPTION OF DRAWINGS

FIG. 1. The PINX1 Gene Encodes a Novel and Conserved Protein.

FIG. 1(A) Full length amino acid sequence of human PinX1 (SEQ ID NO:3).

FIG. 1(B) Domain structure of PinX1. Human PinX1 contains an N-terminal G-patch, a Gly-rich region, and a C-terminal TID domain (Amino acid 254 to Amino acid 328), a telomerase inhibitory domain and Pin2/TRF1-interacting domain.

FIG. 1(C) Human (Hs) PinX1 (SEQ ID NO:3) is a novel protein with sequence homology to ORFs present in other species, including Saccharomyces cerevisiae (Sc) (SCPinX1; (SEQ ID NO:7)) and Caenorhabditis elegans (Ce) (CePinX1; (SEQ ID NO:8)).

FIG. 1(D) cDNA sequence of human PinX1.

FIG. 2. Ubiquitous Expression of Human PINX1mRNA and Identification its Protein.

FIG. 2(A) Expression of PINX1 in human tissues. Human adult tissue Northern blot membranes were probed with PINX1 (top panels), stripped and re-probed with GAPDH for loading control (low panels).

FIG. 2(B) Characterization of anti-PinX1 antibodies. GST-PinX1 was purified and used to immunize rabbits and pre-immune, or immune sera or purified (Pur.) anti-PinX1 antibodies used to perform immunoprecipitation from HeLa cell lysates, followed by immunoblot with anti-PinX1 sera.

FIG. 2(C, D) Detection of endogenous PinX1 and transfected HA-PinX1 proteins. HeLa cells that were not transfected (None) or transfected with the control vector or PinX1 expression construct were subjected to immunoblotting analysis with anti-PinX1 (C) or 12CA5 antibody against the HA tag (D). A sharp arrow points to a non-specific 12CA5-reactive protein.

FIG. 3. Interaction between PinX1 and Pin2/TRF1 in vivo and in vitro.

FIG. 3(A) Co-immunoprecipitation of PinX1 and Pin2/TRF1. HeLa cells were co-transfected with PinX1 and Pin2 expression constructs and then subjected to immunoprecipitation with anti-PinX1 or pre-immune sera, followed by immunoblotting with anti-Pin2 antibodies.

FIG. 3(B) Co-localization of PinX1 with Pin2/TRF1 in cells. HeLa cells were co-transfected with expression constructs of GFP-PinX1 (GREEN) and RFP-Pin2 (RED) and then subjected to fluorescence microscopy after staining DNA with DAPI.

FIG. 3(C, D) Interaction of PinX1 with Pin2/TRF1 in vitro. GST or GST-Pin2 beads (Pin2) were incubated with cell extracts containing HA-PinX1 (C) or with 35S-Pin2 synthesized by in vitro transcription and translation (D). After washing and SDS-PAGE, bound HA-PinX1 was detected by immunoblotting with 12CA antibody and bound 35S-Pin2 by autoradiography.

FIG. 3(E, F) Pin2/TRF1-interacting domain in PinX1. HeLa cells were transfected with various GFP-PinX1 mutants (E) and then subjected to immunoblotting analysis with anti-GFP antibodies directly (Input) or first precipitated by GST or GST-Pin2 beads (Pin2) (F).

FIG. 4. Growth Curves of Stable Cell Lines Expressing PinX1, PinX1-C or PinX1AS.

FIG. 4(A, B) Establishment of stable cell lines expressing PinX1 or PinX1-C or PinX1AS. HT1080 cells were transfected with the control expression vector (vector) or a vector expressing HA-PinX1 or HA-PinX1-C (A) or an antisense PinX1 RNA (PinX1AS) (B). After selection, multiple stable cells were obtained and expression of transgenes was detected by immunoblotting analysis with anti-HA or anti-PinX1 antibodies.

FIG. 4(C, D) Growth curves of stable cell lines. The stable cell lines were maintained continuously in culture, splitting on every fourth day and seeding at the concentration of 6×105 cells per 10 cm culture dish at each subculture. Arrows point to that PinX1-C-expressing cells that have entered crisis.

FIG. 5. Stable Overexpression of PinX1 Induces a Fraction of HT1080 Cells to Enter Senescence-Like State and PinX1-C Forces Most Cells into Crisis.

FIG. 5(A) Reduced cell proliferation induced by PinX1-C and to a lesser extent by PinX1. Cell proliferation of stable cell lines at 28 PD (FIG. 3C) was assayed by labeling cells with BrdU for 30 min in triplicates. Incorporation of BrdU into cells was determined by staining with FITC-labeled anti-BrdU antibodies, followed by flow cytometry.

FIG. 5(B) Apoptosis induced by PinX1-C. After PDB28, a fraction of PinX1-C expressing stable cells were contracted, rounded and loosely attached from culture flasks, which were collected and stained with propidium iodide, followed to flow cytometry to analyze DNA content. Apoptotic cells were detected in these cells, as indicated by sub-G1 DNA content. Note, stable cell lines expressing vector, PinX1 or PinX1AS did not have obvious loosely attached cells.

FIG. 5(C) Senescence-like morphologies induced by PinX1-C and to a lesser extent by PinX1. Cells at 36 PD (FIG. 3C) were fixed and then subjected to senescence-associated β-galactosidase (SA-β-gal) staining, followed by microscopy.

FIG. 6. Overexpression of PinX1 partially and PinX1-C almost completely Inhibits Telomerase Activity, Whereas Depletion of Endogenous PinX1 Increases Telomerase Activity in vivo.

Stable HT1080 cell lines were harvested at 4 PD and telomerase-containing fractions prepared, followed by subjecting different amounts of proteins as indicated to the TRAP assay. Telomerase products were stained with SYBR green (A) and semi-quantified, as described in Experimental Procedures. The average and standard deviation from four experiments are present in (B), with the telomerase activity present in 250 ng extracts prepared from vector control cells being defined as 100%. To present the decrease and increase in telomerase activity induced by modulating PinX1 protein levels, they are presented in two separate panels (B). RNase was included in one assay. Arrows point to the 36 bp internal control (IC) for PCR amplification. Similar results were also obtained with other independent cell lines (not shown).

FIG. 7. PinX1, Pinx-C and PinX1-N bind hTERT, but only PinX1 and PinX1-C Potently Inhibits Telomerase Activity in vitro.

FIG. 7A-C: In vitro interaction of hTERT and PinX1, Pinx-C or PinX1-N. Glutathionne beads containing GST, GST-PinX1 or its N-terminal 142 amino acid fragment (PinX1-N) or C-terminal 74 amino acid fragment (PinX1-C) were incubated with cell extracts containing HA-hTERT (A) or GFP-hTERT (B), or with 35S-hTERT synthesized by in vitro transcription and translation (C). After extensive wash, the bound proteins were separated on SDS-containing gels, followed by detecting HA-hTERT and GFP-hTERT by immunoblot with anti-HA and GFP antibodies, respectively, and 35S-hTERT by autoradiography.

FIG. 7D-G: Potent inhibition of telomerase by PinX1 and PinX1-C, but neither PinX1-N nor the GST tag. Different concentrations of GST or GST-PinX1 (D, G), GST-PinX1-C (E, G), PinX1-N protein (F, G) or His-PinX1 (G) were incubated with telomerase prepared from HT1080 cells for 10 min, followed by the TRAP assay. The average from two experiments was present in (G), with the telomerase activity without protein addition being defined as 100%. Arrows point to the 36 bp internal control (IC) for PCR amplification.

FIG. 8. Functional properties of PinX1 and its mutants. The ability to bind Pin2/TRF1 or hTERT was determined by co-immunoprecipitation and/or GST-pulldown assay. +, binding; −, no binding. The ability to inhibit telomerase activity in vitro and to modulate telomerase in vivo was assayed by the TRAP assay. +, potently inhibition, −no inhibition. The ability to affect cell growth was determined by assaying growth properties, and senescence and/or apoptosis markers. N.D., not determiend; N.A. not applicable.

FIG. 9. Expression of PinX1 in some human tumor tissues as determined by immunostaining.

Human normal or cancer tissues were immunostated with affinity-purified anti-PinX1 antibodies, +, expression readily detectable; −expression significantly reduced as compared with that in normal tissue.

FIG. 10. Depletion of PinX1 by expression of antisense PinX1 increases the tumorigenecity of HT1080 cells. HT1080 cell lines that stably expressed PinX1, PinX1-C, antisense PinX1 (PinX1^{AS}) or control vector were injected to the back of nude mice. The appearance of tumors at the injection sites were monitored weekly, followed by removing the tumors at 8 weeks after injection.

FIG. 11. PinX1-L1 polynucleotide and polypeptide sequences.

FIG. 12. Sequence comparison between PinX1 and PinX1-L1 sequences.

FIG. 13. Pin2 polynucleotide (SEQ ID NO:18) and polypeptide sequences (SEQ ID NO:17).

### SUMMARY OF THE INVENTION

The present invention encompasses an isolated PinX1 polynucleotide comprising or consisting of a sequence of SEQ ID No. 1 or SEQ ID No. 2.

The invention also encompasses an isolated PinX1-L1 polynucleotide comprising or consisting of a sequence of SEQ ID No. 5.

In one embodiment, the above isolated polynucleotide is covalently coupled with a detectable label.

Preferably, the detectable label is one selected from the group consisting of: radiolabel, fluorescent label, chemiluminescent label and colorimetric label.

The invention further encompasses a vector comprising the above mentioned PinX1 nd PinX1-L1 polynucleotides.

The invention further encompasses a host cell comprising the DNA vector comprising the above mentioned PinX1 nd PinX1-L1 polynucleotides.

Preferably, the host cell is a prokaryotic or eukaryotic cell.

The invention encompasses an isolated PinX1 polypeptide comprising or consisting of SEQ ID No. 3 or SEQ ID No. 4.

The invention also encompasses an isolated PinX1-L1 polypeptide comprising or consisting of SEQ ID No. 6.

The invention further encompasses a polyclonal or monoclonal antibody specifically immunoreactive with the above-mentioned PinX1 or PinX1-L1 polypeptide and the hybridoma cell lines for producing said monoclonal antibodies.

Preferably, the antibody is covalently coupled with a detectable label.

More preferably, the detectable label is one selected from the group consisting of: radiolabel, fluorescent label, chemiluminescent label and colorimetric label.

The invention provides a method for diagnosis of a cancerous or precancerous condition in a mammal, said method comprising performing a detection step to detect a hybrid formed between a probe and a biological sample from said mammal, said probe comprising a sequence complementary to 15 or more (e.g., 20, 25, 30, 35, 40, 45, 50, 100, 200, 300, 400, 500, or more) consecutive nucleotide sequence of SEQ ID No. 1 or SEQ ID No. 5, wherein the absence of a detectable hybrid is indicative of said cancerous or precancerous condition.

In one embodiment, the method for dignosis further comprises the step of comparing the amount of said hybrid detected in said biological sample with the amount of a control hybrid detected comprising said probe and a target polynucleotide comprising SEQ ID No. 1 or SEQ ID No. 5 in a control sample, wherein a reduction of the amount of detectable hybrid relative to said control hybrid is indicative of said cancerous or precancerous condition.

Preferably, the probe used in said method for diagnosis is covalently coupled with a detectable label.

More preferably, said detectable label is one selected from the group consisting of: radiolabel, fluorescent label, chemiluminescent label, and calorimetric label.

The invention also provides a method for diagnosis of a cancerous or precancerous condition in a mammal, said method comprising performing a detection step to detect an amplification of 50 or more (e.g., 60, 70, 80, 90, 100, 150, 200, 250, 300, 400, 500, or more) consecutive nucleotide sequence of SEQ ID No. 1 or SEQ ID No. 5 in a biological sample from said mammal using one or more of primers, each said primer being complementary to said consecutive nucleotide sequence, wherein an absence of said amplification is indicative of said cancerous or precancerous condition.

In one embodiment, said method for diagnosis further comprises the step of comparing the amount of said amplification detected in said biological sample with the amount of a control amplification detected comprising said primers and a target polynucleotide comprising SEQ ID No. 1 or SEQ ID No. 5 of a control sample, wherein a reduction of the amount of said amplification relative to said control amplification is indicative of said cancerous or precancerous condition.

In one embodiment, said amplification is by a polymerase chain reaction.

The invention provides a method for diagnosis of a cancerous or precancerous condition in a mammal, said method comprising performing a detection step to detect the formation of a complex between an antibody and a polypeptide comprising SEQ ID No. 3, SEQ ID No. 4 or SEQ ID No. 6 in a biological sample from said mammal, wherein an absence of the formation of said complex is indicative of said cancerous or precancerous condition.

In one embodiment, said method for diagnosis further comprises the step of comparing the amount of said complex detected in said biological sample with the amount of a control complex detected comprising said antibody and a target polypeptide comprising SEQ ID No. 3, SEQ ID No. 4 or SEQ ID No. 6 of a control sample, wherein a reduction of the amount of said complex relative to the amount of said control complex is indicative of said cancerous or precancerous condition.

In some embodiments of the invention, said antibody used for said diagnosis is covalently coupled with a detectable label.

Preferably, said detectable label is one selected from the group consisting of: radiolabel, fluorescent label, chemiluminescent label and calorimetric label.

In some embodiments of the invention, said cancerous condition is selected from a solid tumor and a leukemia.

In some embodiments of the invention said mammal is human.

The invention further provides a method for reducing telomerase function in an eukaryotic cell comprising contacting said eukaryotic cell with a polynucleotide comprising SEQ ID No. 1, SEQ ID No. 2 or SEQ ID No. 5, and expressing said polynucleotide in said eukaryotic cell in an amount sufficient to reduce telomerase function.

The invention also provides a method for reducing telomerase function in an eukaryotic cell comprising contacting said eukaryotic cell with a polypeptide comprising SEQ ID No. 3, SEQ ID No. 4 or SEQ ID No. 6 in an amount sufficient to reduce telomerase function.

In some embodiment, said reduction of telomerase function is determined by measuring one or more of: a reduction in telomerase enzymatic activity, a reduction in telomere length, a reduction in cell proliferation, an induction of senescence, and an induction of crisis in said cell.

Preferably, said eukaryotic cell is a mammalian cell.

More preferably, said mammalian cell is a human cell.

The present invention provides a method for preventing or treating a cancerous condition in a mammal comprising administering a therapeutically effective amount of a polynucleotide comprising SEQ ID No. 1, SEQ ID No. 2 or SEQ ID No. 5.

The invention also provides a method for preventing or treating a cancerous condition in a mammal comprising administering a therapeutically effective amount of a polypeptide comprising SEQ ID No. 3, SEQ ID No. 4 or SEQ ID No. 6.

In one embodiment, said therapeutically effective administration results in a reduction in tumor size.

In another embodiment, said therapeutically effective administration results in a reduction in number of tumor cells.

In one embodiment, said mammal in the method for preventing or treating a cancerous condition is a human.

In another embodiment, said polynucleotide or polypeptide is administered a method for preventing or treating a cancerous condition as a pharmaceutical composition further comprising a pharmaceutically acceptable carrier.

The invention provides a method for increasing telomerase function in an eukaryotic cell comprising contacting said eukaryotic cell with a polynucleotide comprising an antisense polynucleotide complementary to the corresponding mRNA sequence comprising SEQ ID No. 1 or SEQ ID No. 5 in an sufficient amount to increase telomerase function.

The invention also provides a method of increasing telomerase function in an eukaryotic cell comprising contacting said eukaryotic cell with an antibody in an sufficient amount to increase telomerase function, said antibody being specifically immunoreactive with a polypeptide comprising SEQ ID No. 3 or SEQ ID No. 6.

In one embodiment, the increase of telomerase function in the method of increasing telomerase function is determined by measuring one or more of: an increase in telomerase enzymatic activity, an increase or maintenance in telomere length, an increase in cell proliferation, a reduction of senescence and a reduction of crisis in said cell.

In one embodiment, said eukaryotic cell is a mammalian cell.

In another embodiment, said mammalian cell is a human cell.

The invention further provides a method for preventing aging in a mammal comprising administering a therapeutically effective amount of an antisense polynucleotide complementary to the corresponding mRNA sequence comprising SEQ ID No. 1 or SEQ ID No. 5.

The invention also provides a method for preventing aging in a mammal comprising administering a therapeutically effective amount of an antibody, wherein said antibody is specifically immunoreactive with a polypeptide comprising SEQ ID No. 3 or SEQ ID No. 6.

In another embodiment, said mammalian cell in the method for preventing aging is a human cell.

In one embodiment, said antisense polynucleotide or antibody used in the method for preventing aging is administered as a pharmaceutical composition further comprising a pharmaceutically acceptable carrier.

The invention provides a pharmaceutical composition comprising a therapeutically effective amount of a polynucleotide comprising SEQ ID No. 1, SEQ ID No. 2 or SEQ ID No. 5.

The invention also provides a pharmaceutical composition comprising a therapeutically effective amount of a polypeptide comprising SEQ ID No. 3, SEQ ID No. 4 or SEQ ID No. 6.

The invention further provides a pharmaceutical composition comprising a therapeutically effective amount of an antibody specifically immunoreactive with a polypeptide comprising SEQ ID No. 3 or SEQ ID No. 6.

The invention further provides a pharmaceutical composition comprising a therapeutically effective amount of an antisense oligonucleotide complementary to the corresponding mRNA sequence comprising SEQ ID No. 1 or SEQ ID No. 5.

In one embodiment, the above-mentioned pharmaceutical composition further comprises a pharmaceutically acceptable carrier.

The invention provides a method for screening for an agent which modulates the binding between a polypeptide (SEQ ID No. 3, SEQ ID No. 4 or SEQ ID No. 6) and a Pin 2 polypeptide, said method comprising:

(a) incubating a mixture comprising said polypeptide (SEQ ID No. 3 or SEQ ID No. 4), a Pin2 polypeptide, and a candidate agent, wherein said incubating whereby, but for the presence of said agent, allows said polypeptide (SEQ ID No. 3, SEQ ID No. 4 or SEQ ID No. 6) to bind to said Pin2 polypeptide to form a complex;

(b) detecting said complex formation in (a); and

(c) comparing said complex detected in (b) with a control comprising said polypeptide (SEQ ID No. 3, SEQ ID No. 4 or SEQ ID No. 6) and said Pin2 polypeptide in the absence of a candidate agent, wherein an absence, an increase, or a reduction of said complex detected in (b) is indicative of said candidate agent modulating the binding activity of said polypeptide (SEQ ID No. 3, SEQ ID No. 4 or SEQ ID No. 6) to said Pin2 polypeptide.

The invention also provides a method for screening for an agent which modulates the binding between a polypeptide comprising SEQ ID No. 3, SEQ ID No. 4 or SEQ ID No. 6 and a Pin 2 polypeptide in an eukaryetic cell, said method comprising:

(a) contacting said eukaryotic cell with a candidate agent, wherein said contacting whereby, but for the presence of said agent, allows said polypeptide comprising SEQ ID No. 3 SEQ ID No.4 or SEQ ID No. 6 to bind to said Pin2 polypeptide to form a complex in said cell;

(b) detecting said complex formation in (a); and

(c) comparing said complex detected in (b) with a control cell without contacting said control cell to said candidate agent, wherein an absence, an increase, or a reduction of said complex formation in (b) is indicative of said candidate agent modulating the binding activity of said polypeptide comprising SEQ ID No. 3 or SEQ ID No. 4 or SEQ ID No. 6 to said Pin2 polypeptide.

The invention provides a method for screening for an agent which modulates the binding between a polypeptide (SEQ ID No. 3, SEQ ID No. 4 or SEQ ID No. 6) and a telomerase polypeptide, said method comprising:

(a) incubating a mixture comprising said polypeptide (SEQ ID No. 3 or SEQ ID No. 4), a telomerase polypeptide, and a candidate agent, wherein said incubating whereby, but for the presence of said agent, allows said polypeptide (SEQ ID No. 3, SEQ ID No. 4 or SEQ ID No. 6) to bind to said telomerase polypeptide to form a complex;

(b) detecting said complex formation in (a); and

(c) comparing said complex detected in (b) with a control comprising said polypeptide (SEQ ID No. 3, SEQ ID No. 4 or SEQ ID No. 6) and said telomerase polypeptide in the absence of a candidate agent, wherein an absence, an increase, or a reduction of said complex detected in (b) is indicative of said candidate agent modulating the binding activity of said polypeptide (SEQ ID No. 3, SEQ ID No. 4 or SEQ ID No. 6) to said telomerase polypeptide.

The invention also provides a method for screening for an agent which modulates the binding between a polypeptide comprising SEQ ID No. 3, SEQ ID No. 4 or SEQ ID No. 6 and a telomerase polypeptide in an eukaryotic cell, said method comprising:

(a) contacting said eukaryotic cell with a candidate agent, wherein said contacting whereby, but for the presence of said agent, allows said polypeptide comprising SEQ ID No. 3 SEQ ID No. 4 or SEQ ID No. 6 to bind to said telomerase polypeptide to form a complex in said cell;

(b) detecting said complex formation in (a); and

(c) comparing said complex detected in (b) with a control cell without contacting said control cell to said candidate agent, wherein an absence, an increase, or a reduction of said complex formation in (b) is indicative of said candidate agent modulating the binding activity of said polypeptide comprising SEQ ID No. 3 or SEQ ID No. 4 or SEQ ID No. 6 to said telomerase polypeptide.

In some embodiment, said complex detection in the above methods is through an antibody, said antibody being specifically immunoactive to a polypeptide comprising SEQ ID No. 3 or SEQ ID No. 6.

In a preferred embodiment, said antibody used for detecting said complex formation is covalently coupled with a detectable label.

In a more preferred embodiment, said detectable label is one selected from the group consisting of: radiolabel, fluorescent label, chemiluminescent label, and colorimetric label.

The invention provides a method for screening for an agent which modulates the expression of a polynucleotide comprising SEQ ID No. 1 or SEQ ID No. 5 in an eukaryotic cell, said method comprising:

(a) contacting said eukaryotic cell with a candidate agent;

(b) detecting the expression of said polynucleotide in said eukaryotic cell; and

(c) comparing the expression of said polynucleotide in (b) with a control cell without contacting said control cell to said candidate agent, wherein an increase or a decrease of the expression of said polynucleotide in (b) is indicative of said candidate agent modulating the expression of said polynucleotide.

In one embodiment, said expression detection is through a probe or a pair of primers, each said probe or primer having a sequence complementary to the sequence of said polynucleotide.

In a preferred embodiment, said expression detection is by a polymerase chain reaction.

In another embodiment, said expression detection is through an antibody, said antibody being specifically immunoactive to a polypeptide comprising SEQ ID No. 3 or SEQ ID No. 6.

In a preferred embodiment, said polynucleotide or said antibody used for expression detection is covalently coupled with a detectable label.

Preferably, said detectable label is one selected from the group consisting of: radiolabel, fluorescent label, chemiluminescent label, and colorimetric label.

The present invention provides a method for screening for an agent as a binding partner to a Pin2 polypeptide comprising SEQ ID No. 8, said method comprising:

(a) incubating a mixture comprising said Pin2 polypeptide and a candidate agent, wherein said incubating allows said Pin2 polypeptide to bind to its binding partners to form a complex; and

(b) detecting said complex formation between said Pin2 polypeptide and said candidate, wherein a presence of said complex formation is indicative of said candidate agent being a binding partner to said Pin2 polypeptide.

The present invention also provides a method for treating a cancerous condition in a mammal comprising administering a therapeutically effective amount of an agent which enhances the binding between a PinX1 polypeptide comprising SEQ ID No. 3 or SEQ ID No. 4 or SEQ ID No. 6 to a Pin2 polypeptide, wherein said administration restores the binding between said PinX1 polypeptide and said Pin2 polypeptide to a normal level.

The present invention further provides a method for treating a cancerous condition in a mammal comprising administering a therapeutically effective amount of an agent which increases the expression of a PinX1 polynucleotide comprising SEQ ID No. 1 or SEQ ID No. 5, wherein said administration restores the expression of said PinX1 polynucleotide to that of a normal level.

In some embodiments of the invention, said therapeutically effective administration results in a reduction in tumor size.

The other embodiments of the invention, said therapeutically effective administration results in a reduction in number of tumor cells.

### DETAILED DESCRIPTION OF THE INVENTION

Definitions

As used herein, “telomerase activity” refers to the ability of telomerase protein components to function either in vivo or in vitro into as part of a multi-component enzyme that elongates telomeric DNA. A preferred assay method for detecting telomerase activity is the TRAP assay (see also the commercially available TRAP-eze™ telomerase assay kit (Oncor); and Morin, 1989, Cell 59:521-529). This assay measures the amount of radioactive or non-radiaoactive labeled nucleotides incorporated into elongation products, polynucleotides, formed by nucleotide addition to a telomerase substrate or primer. The radioactivity or non-radioactive signals incorporated can be measured by methods well known in the art (e.g., using the Phosphorlmager™ screens for radio active labels). A test sample and a control sample can be compared. According to the invention, a 10%, 15%, 20% 25% 30%, 40%, 50% or higher, up to 5 fold, 10 fold, 20 fold or higher difference of the telomerase activity between the test sample and the control sample indicates the modulation of telomerase activity in said test sample.

As used herein, the term “senescence” is meant the loss of ability of a cell to replicate in the presence of normally appropriate cell replicative signals, and may be associated with the expression of senescence associated proteins, such as collagenase or senescence-associated β-galactosidase (Drmri et al., 1995; Shay et al., 1992, The two-stage mechanism controlling cellular senescence and immortalization. Exp Gerontol. 1992 Jul-Aug; 27(4):383-9). Senescense corelates well with a decrease of telomere length. According to the invention, the term “induction of senescence” means the inhibition of cell replication ability by inhibiting telomerase function, while the term “reduction of senescence” means increasing cell replication ability by enhancing telomerase function. According to the invention, a 5% or more (e.g., 10%, 15%, 20%, 30%, 40%, 50%, up to 2 fold, 5 fold, 10 fold or more) difference of a senescence marker (e.g., expression of a senescence-associated gene) between a test sample and a control sample is indicative a change or modulation of cell senescence in said test sample.

As used herein, a “senescence marker” refers to a characteristics exhibited by cells in senescence. Useful “senescence marker” according to the invention include, but are not limited to: cell morphology, senescence-associated gene, G1 arrest in cell cycle. Methods for examining senescence markers are well known in the art and examples are provided in the present invention.

“Senescent gene expression” refers to the expression of genes and gene products that are differentially expressed in a senescent as opposed to a young cell. Senescent gene expression can be altered by increasing the expression of young cell specific genes and/or decreasing expression of senescent cell specific genes. These cell specific genes are also denoted as “senescence-related genes”. The proteins encoded by the senescence-related genes are also referred to herein as “senescence-assoiated proteins.”

As used herein, the term “crisis” or “M2 senescence” refers to a state in a cell caused by shortening of telomeres (Shay et al., 1992, The two-stage mechanism controlling cellular senescence and immortalization. Exp Gerontol. 1992 Jul-Aug; 27(4):383-9). Most cells die in crisis. Rarely, mutants may occur to reactivates telomerase and stabilizes the telomere length so that a cell may past crisis and become immortal. According to the invention, a 5% or more (e.g., 10%, 15%, 20%, 30%, 40%, 50%, up to 2 fold, 5 fold, 10 fold or more) difference of a crisis marker between a test sample and a control sample is indicative a change or modulation of cell senescence in said test sample.

As used herein, a “crisis marker” refers to a characteristics exhibited by cells in crisis. Useful “cirsis marker” according to the invention include, but are not limited to: reduction of cell proliferation, cell morphology, cell cycle profile). Methods for examining crisis markers are well known in the art and examples are provided in the present invention.

As used herein, the term “proliferation” refers to the rate of cell division and the ability of a cell to continue to divide. One complete cell division process is referred to as a “cycle”. By an “increase in cell proliferation” is meant to increase the cell division rate so that the cell has a higher rate of cell division compared to normal cells of that cell type, or to allow the cell division to continue for more cycles without changing the rate of each cell division. By an “decrease in cell proliferation” is meant to decrease the cell division rate so that the cell has a lower rate of cell division compared to normal cells of that cell type, or to reduce the number of cycles of the cell division without changing the rate of each cell division. According to the invention, a 10% or higher (e.g., 20%, 30%, 40% 50%, up to 2 fold, 5 fold, 10 fold or higher) difference in cell proliferation between a test sample and a control sample is indicative of a change or a modulation in proliferation of said test sample.

As used herein, “binding” refers to the ability of a given polypeptide (e.g., PinX1) to associate with another polypeptide (e.g., Pin2) through specific amino acid side chain interaction. Therefore, the term “binding” does not encompass non-specific binding, such as non-specific adsorption to a surface. Non-specific binding can be readily identified by including the appropriate controls in a binding assay. As used herein, “binding partner” means a molecule or an agent which specifically binds a Pin2 protein or a telomerase polypeptide.

The term “expression modulation” or “modulate the expression” refers to the capacity of an agent to either enhance or inhibit transcription or translation of a polynucleotide sequence.

As used herein, the term “polynucleotide(s)” generally refers to any polyribonucleotide or poly-deoxyribonucleotide, which may be unmodified RNA or DNA or modified RNA or DNA. “Polynucleotide(s)” include, without limitation, single- and double-stranded nucleic acids. As used herein, the term “polynucleotide(s)” also includes DNAs or RNAs as described above that contain one or more modified bases. Thus, DNAs or RNAs with backbones modified for stability or for other reasons are “polynucleotide(s)”. The term “polynucleotide(s)” as it is employed herein embraces such chemically, enzymatically or metabolically modified forms of polynucleotides, as well as the chemical forms of DNA and RNA characteristic of viruses and cells, including, for example, simple and complex cells. “Polynucleotide(s)” also embraces short polynucleotides often referred to as oligonucleotide(s). A polynucleotide according to the invention may vary from 10 bp to 10 kb, or 100 kb or more in length and may be single or double stranded. A DNA polynucleotide according to the invention may be a cDNA or a genomic DNA or a recombinant DNA. For example, an amplified or assembled DNA may be inserted into a suitable DNA vector, such as a bacterial plasmid or a viral vector, and the vector can be used to transform or transfect a suitable host cell. The gene is then expressed in the host cell to produce the recombinant protein. A recombinant DNA may serve a non-coding function (e.g., promoter, origin of replication, ribosome-binding site, etc.) as well.

As used herein, “deletion” refers to a change in either a nucleotide or amino acid sequence wherein one or more nucleotides or amino acid residues, respectively, are absent.

As used herein, “insertion” or “addition” refers to a change in either nucleotide or amino acid sequence wherein one or more nucleotides or amino acid residues, respectively, have been added.

As used herein, “substitution” refers to a replacement of one or more nucleotides or amino acids by different nucleotides or amino acid residues, respectively.

“Polypeptide” and “protein” are used interchangeably herein to refer to a polymer of amino acid residues. The term “recombinant protein” refers to a protein that is produced by expression of a recombinant DNA molecule that encodes the amino acid sequence of the protein. Polynucleotides and recombinantly produced polypeptide, and fragments or analogs thereof, may be prepared according to methods known in the art and described in Maniatis et al., Molecular Cloning: A Laboratory Manual, 2nd Ed., (1989), Cold Spring Harbor, N.Y., and Berger and Kimmel, Methods in Enzymology, Volume 152, Guide to Molecular Cloning Techniques (1987), Academic Press, Inc., San Diego, Calif., which are incorporated herein by reference.

As used herein, the term “open reading frame” refers to a polynucleotide sequence that encodes a polypeptide and is bordered on the 5′-end by an initiation codon (ATG) or another codon that does not encode a stop codon and on the 3′-end by a stop codon but otherwise does not contain any in-frame stop codons between the codons at the 5′-border and the 3′-border.

As used herein the term “encoding” refers to the inherent property of specific sequences of nucleotides in a polynucleotide, such as a gene in a chromosome or an mRNA, to serve as templates for synthesis of other polymers and macromolecules in biological processes having a defined sequence of nucleotides (i.e., rRNA, tRNA, other RNA molecules) or amino acids and the biological properties resulting therefrom. Thus a gene encodes a protein, if transcription and translation of mRNA produced by that gene produces the protein in a cell or other biological system. Both the coding strand, the nucleotide sequence of which is identical to the mRNA sequence and is usually provided in sequence listings, and non-coding strand, used as the template for transcription, of a gene or cDNA can be referred to as encoding the protein or other product of that gene or cDNA. A polynucleotide that encodes a protein includes any polynucleotides that have different nucleotide sequences but encode the same amino acid sequence of the protein due to the degeneracy of the genetic code. Polynucleotides and nucleotide sequences that encode proteins may include introns and may be genomic DNA.

As used herein, “cDNA” refers to deoxyribonucleic acids produced by reverse-transcription and typically second-strand synthesis of mRNA or other RNA produced by a gene; if double-stranded, a cDNA molecule has both a coding or sense and a non-coding or antisense strand.

As used herein, the term “complementary to” refers to a polynucleotide sequence that can hybridize specifically to another polynucleotide sequence; for example, a nucleic acid comprising nucleotides in the sequence “5′-TATAC” is complementary to a nucleic acid comprising nucleotides in the sequence “5′-GTATA”.

As used herein, the term “specific hybridization” refers to the formation, by hydrogen bonding or nucleotide (or nucleobase) bases, of hybrids between a probe polynucleotide (e.g., a polynucleotide complementary to SEQ ID No. 1 of the invention and a specific target polynucleotide (e.g., SEQ ID No. 1 or its mRNA sequence of the invention), wherein the probe preferentially hybridizes to the specific target such that, for example, a single band corresponding to said hybridization can be identified on a Southern blot or a Northern blot of DNA or RNA prepared from a suitable source (e.g., cells from a cancer patient).

As used herein, the term “corresponds to” or “corresponding” refers to (i) a polynucleotide having a nucleotide sequence that is complementary to all or a fragment comprising 10 or more consecutive nucleotides of a reference polynucleotide sequence or encoding an amino acid sequence at least 70%, preferably 80%, more preferably 90% identical to an amino acid sequence in a peptide or protein; or (ii) a peptide or polypeptide having an amino acid sequence that is at least 70%, preferably 80%, more preferably 90% identical to at least 15 or more consecutive amino acid sequence in a reference peptide or protein. For example, a “corresponding mRNA sequence of SEQ ID No. 1” refers to a mRNA molecule transcribed from a polynucleotide comprising SEQ ID No. 1 and being complementary to SEQ ID No. 1.

An “expression control sequence” refers to a nucleotide sequence in a nucleic acid that regulates the expression (transcription and/or translation) of a nucleotide sequence operatively linked thereto. Expression control sequences can include, for example and without limitation, sequences of a promoter, enhancer, and transcription terminator, all of which can be involved in transcription of DNA to form RNA, and a ribosome-binding site, start codon (i.e., ATG), splicing signal for an intron/exon, and a stop codon, all of which can be involved in translation of RNA to form a protein.

As used herein, “primer” refers to a polynucleotide, i.e., a purified restriction fragment or a synthetic polynucleotide, that is capable of acting as a point of initiation of synthesis when placed under conditions in which synthesis of a primer extension product complementary to a polynucleotide strand (the “template”) is induced, i.e., in the presence of nucleotides and an agent for polymerization such as DNA polymerase and at a suitable temperature and pH. The primer is preferably single-stranded for maximum efficiency in amplification but may alternatively be double-stranded. If double stranded, the primer may need to be treated to separate its strands before being used to prepare extension products. A primer must be sufficiently long to prime the synthesis of extension products in the presence of the agent for polymerization. The length of a primer depends on many factors, including application, temperature to be employed, template, reaction conditions, other reagents, and source of primers. For example, depending on the complexity of the target sequence, the polynucleotide primer typically contains 15-200 or more nucleotides, although it may contain fewer nucleotides or up to several kilobases or more.

As used herein, “probe” refers to a molecule that binds to a specific sequence or subsequence or other moiety of another molecule. Unless otherwise indicated, the term “probe” typically refers to a polynucleotide probe that binds to another polynucleotide, often called the “target nucleic acid”, through complementary base pairing. Probes may bind target nucleic acids lacking complete sequence complementarity with the probe, depending upon the stringency of the hybridization conditions. A probe according to the invention is 25 to 5000 nucleotides, more preferably 50 to 250 nucleotides in length. The probe may be single or double stranded.

“Antisense” refers to oligonuleotides or polynucleotides comprising sequences of nucleotides that are complementary to a sequence in another oligonucleotide or polynucleotide (e.g., mRNA). Antisense oligonucleotides can be produced by a variety of methods, as is commonly known in the art. For example, but not limitation, antisense RNA can be synthesized by splicing the gene(s) or coding sequence of a gene of interest in a reverse orientation, relative to its orientation in nature, to a promoter that directs the synthesis of the antisense nucleic acid. An antisense oligonucleotide can bind to a complementary sequence in its “target” nucleic acid, such as a naturally occurring mRNA produced by a cell, via hydrogen bonding to form a duplex or double-stranded nucleic acid. Such duplex formation can reduce or completely inhibit the translation of proteins from the target mRNA or, if the antisense oligonucleotide is bound to DNA in a gene, transcription of that gene. In this manner, alteration or modulation of gene expression can be achieved.

As used herein, the term “antibody” refers to naturally occurring and recombinant polypeptides and proteins encoded by immunoglobulin genes, or fragments thereof, that specifically bind to or “recognize” an analyte or “antigen”. Immunoglobulin genes include the kappa, lambda, alpha, gamma, delta, epsilon and mu constant region genes, as well as myriad immunoglobulin variable region genes. An antibody can exist as an intact immunoglobulin or as any one of a number of well characterized fragments, e.g., Fab′ and F(ab)′₂ fragments, produced by various means, including recombinant methodology and digestion with various peptidases. An “antibody” according to the invention may be a polyclonal or a monoclonal antibody.

As used herein, “specifically immunoreactive” refers to the ability of an antibody to contact and associate its corresponding antigen. Thus, under designated immunoassay conditions, an antibody binds preferentially to a particular protein and not in a significant amount to other proteins present in the sample. Specific binding to a protein under such conditions requires an antibody selected for its specificity for a particular protein. To select antibodies specifically immunoreactive with a particular protein, one can employ a variety of means, i.e., solid-phase ELISA immunoassays are routinely used to select monoclonal antibodies specifically immunoreactive with a protein. See Harlow and Lane (1988), Antibodies, A Laboratory Manual, Cold Spring Harbor Publications, New York.

An “immunoassay” refers to an assay in which an antibody or fragment thereof is used to detect an analyte.

As used herein, “stringent condition” refers to temperature or ionic condition used in nucleic acid hybridization. The stringency required is nucleotide sequence dependent and also depends upon the various components present during hybridization. Generally, stringent conditions are selected to be about 5 to 20 degrees C. lower than the thermal melting point (Tm) for the specific sequence at a defined ionic strength and pH. The Tm is the temperature (under defined ionic strength and pH) at which 50% of a target sequence hybridizes to a complementary probe.

As used herein, the term “agent” refers to a molecule selected from the group consisting of a chemical compound, a polynucleotide, a polypeptide, or an antibody. A “agent” may exist as a mixture of molecules, an array of spatially localized molecules (e.g., a polypeptide array, polynucleotide array, and/or combinatorial small molecule array), a library, or an extract made from biological materials such as mammalian cells or tissues. The invention provides an “agent” that 1) modulates the binding between a PinX1 polypeptide and a Pin2 polypeptide; 2) modulates the expression of a PinX1 polynucleotide.

The term “endogenous DNA sequence” refers to naturally-occurring polynucleotide sequences contained in a eukaryotic cell. Such sequences include, for example, chromosomal sequences (e.g., structural genes, promoters, enhancers, recombinatorial hotspots, repeat sequences, integrated proviral sequences). An “exogenous polynucleotide” is a polynucleotide which is transferred into an eukaryotic cell.

As used herein, “sequence identity” refers to sequences that are identical (i.e., on a nucleotide-by-nucleotide or amino acid-by-amino acid basis) over the window of comparison. The term “percentage of sequence identity” is calculated by comparing two optimally aligned sequences over the window of comparison, determining the number of positions at which the identical nucleic acid base (e.g., A, T, C, G, U, or I) occurs in both sequences to yield the number of matched positions, dividing the number of matched positions by the total number of positions in the window of comparison (i.e., the window size), and multiplying the result by 100 to yield the percentage of sequence identity.

A “window of comparison” refers to a conceptual segment of typically at least 12 contiguous residues that is compared to a reference sequence; the window of comparison may comprise additions or deletions (i.e., gaps) of about 20 percent or less as compared to the reference sequence (which does not comprise additions or deletions) for optimal alignment of the two sequences. Optimal alignment of sequences for aligning a window of comparison may be conducted by computerized implementations of algorithms (GAP, BESTFIT, FASTA, and TFASTA in the Wisconsin Genetics Software Package Release 7.0, Genetics Computer Group, 575 Science Dr., Madison, Wis.) or by inspection, and the best alignment (i.e., resulting in the highest percentage of homology over the comparison window) generated by any of the various methods is selected.

As used herein, “isolated” when used in reference to a nucleic acid means that a naturally occurring sequence has been removed from its normal cellular (e.g., chromosomal) environment or is synthesized in a non-natural environment (e.g., artificially synthesized). Thus, an “isolated” sequence may be in a cell-free solution or placed in a different cellular environment. An “isolated DNA” may be DNA free of the genes that flank the gene of interest in the genome of the organism in which the gene of interest naturally occurs. The term therefore includes a recombinant DNA incorporated into a vector, into an autonomously replicating plasmid or virus, or into the genomic DNA of a prokaryote or eukaryote. It also includes a separate molecule such as a cDNA, a genomic fragment, a fragment produced by polymerase chain reaction (PCR), or a restriction fragment. It also includes a recombinant nucleotide sequence that is part of a hybrid gene, i.e., a gene encoding a fusion protein. Also included is a recombinant DNA that includes a portion of SEQ ID No. 1 or SEQ ID No. 5 and that encodes an alternative splice variant of SEQ ID No. 1 or SEQ ID No. 5.

The term “naturally occuring” refers to a molecule, typically an amino acid, nucleotide, polynucleotide, or polypeptide, that exists in nature without human intervention. In contradistinction, the term “recombinant” refers to a molecule listed above herein that exists only with human intervention.

As used herein, the term “operably linked” refers to a linkage of polynucleotide elements in a functional relationship. A nucleic acid is “operably linked” when it is placed into a functional relationship with another nucleic acid sequence. For instance, a promoter or enhancer is operably linked to a coding sequence if it affects the transcription of the coding sequence. Operably linked means that the DNA sequences being linked are typically contiguous and, where necessary to join two protein coding regions, contiguous and in reading frame. However, since enhancers generally function when separated from the promoter by several kilobases and intronic sequences may be of variable lengths, some polynucleotide elements may be operably linked but not contiguous. A structural gene (e.g., a PinX1 gene) which is operably linked to a polynucleotide sequence corresponding to a transcriptional regulatory sequence of an endogenous gene is generally expressed in the same temporal and cell type-specific pattern as is the naturally-occurring gene.

As used herein, the term “transcriptional unit” or “transcriptional complex” refers to a polynucleotide sequence that comprises a structural gene (exons), a cis-acting linked promoter and other cis-acting sequences necessary for efficient transcription of the structural sequences, distal regulatory elements necessary for appropriate tissue-specific and developmental transcription of the structural sequences, and additional cis sequences important for efficient transcription and translation (e.g., polyadenylation site, mRNA stability controlling sequences).

As used herein, the term “label” refers to a detectable marker and to the incorporation of such a marker into a polynucleotide, an antibody, or other molecule. The label may be a radioisotope (e.g., .³H, ¹⁴C., ³⁵S, ¹²⁵I, ¹³¹I), a fluorescent dye (e.g., fluorescein, rhodamine phycoerythin, phycocyanin, allophycocyanin, and fluorescamine), or chemiluminescent molecule (e.g., luminal, isoluminal, aromatic acridinium ester, imidazole, acridinium salt, oxalate ester, luciferin, luciferase, and aequorin), or an enzyme (e.g., horseradish peroxidase, β-galactosidase, luciferase, alkaline phosphatase).

As used herein, “host cell” refers to a cell that comprises a recombinant polynucleotide molecule, typically a recombinant plasmid or other expression vector. Thus, for example, host cells can express genes that are not found within the native (non-recombinant) form of the cell. The host cell may be prokaryotic or eukaryotic, including bacterial, mammalian, yeast, Aspergillus, and insect cells.

As used herein, the term “pharmaceutical composition” refers to a composition suitable for pharmaceutical use in a mammal. A pharmaceutical composition comprises a pharmacologically effective amount of an active agent and a pharmaceutically acceptable carrier. “Pharmacologically effective amount” or “therapeutically effective amount” refers to that amount of an agent effective to produce the intended pharmacological result. “Therapeutically effective”, according to the invention, refers to a modulation of telomerase function by at least 10%, for example, 20%, 30%, 40% or higher, up to 2 fold, 5 fold, 10 fold or higher. “Therapeutically effective” also refers to a reduction of tumor size or a reduction in the number of tumor cells by at least 5%, for example, 10%, 15%, 20%, 25%, 30%, 35%, 40%, 45%, 50%, 60%, 70%, 80%, 90% or more, up to 100%. “Pharmaceutically acceptable carrier” refers to any of the standard pharmaceutical carriers, buffers, and excipients, such as a phosphate buffered saline solution, 5% aqueous solution of dextrose, and emulsions, such as an oil/water or water/oil emulsion, and various types of wetting agents and/or adjuvants or diluents. Suitable pharmaceutical carriers and formulations are described in Remington's Pharmaceutical Sciences, 19th Ed. (Mack Publishing Co., Easton, 1995). Preferred pharmaceutical carriers depend upon the intended mode of administration of the active agent. Typical modes of administration include enteral (i.e., oral) or parenteral (i.e., subcutaneous, intramuscular, or intravenous intraperitoneal injection; or topical, transdermal, or transmucosal administration).

As used herein, the term “disease allele” refers to an allele of a gene which is capable of producing a recognizable disease. A disease allele may be dominant or recessive and may produce disease directly or when present in combination with a specific genetic background or pre-existing pathological condition. A disease allele may be present in the gene pool or may be generated de novo in an individual by somatic mutation.

As defined herein, “an individual” is a single organism and includes humans, animals, plants, multicellular and unicellular organisms.

As used herein, the term “cancer” refers to a malignant disease caused or characterized by the proliferation of cells which have lost susceptibility to normal growth control. “Malignant disease” refers to a disease caused by cells that have gained the ability to invade either the tissue of origin or to travel to sites removed from the tissue of origin.

As defined herein a “a biological sample” is a material suspected of comprising an analyte and includes a biological fluid, suspension, buffer, collection of cells, fragment or slice of tissue. A biological fluid includes blood, plasma, sputum, urine, and cerebrospinal fluid.

The invention is based upon the discovery of two Pin2 polypeptides PinX1 and PinX1-L1 (SEQ ID No. 3 and SE ID NO. 6 respectively) and polynucleotides encoding such polypeptides

PinX1 polypeptide inhibits telomerase activity in vitro and in vivo and influences cell growth. PinX1 co-immunoprecipitates and co-localizes with the human telomere binding protein Pin2/TRF1 in cells. Importantly, both PinX1 and its small C-terminal TID domain (SEQ ID No. 4) interact with the telomerase catalytic subunit hTERT and potently inhibit its activity in vitro, with an IC50 of ˜50 nM. When stably expressed in the human fibrosarcoma cell line HT1080, PinX1 significantly inhibits cellular telomerase activity and strikingly, its c-terminal TID domain (amino acids 254-328) almost completely inhibits telomerase activity and also forces the tumor cells into crisis. In contrast, depletion of endogenous PinX1 by expression of an antisense PinX1 RNA significantly increases telomerase activity in HT1080 cells. Interestingly, the human PINX1 gene is located at 8p23, a region with frequent loss of heterozygosity in a number of human cancers. Thus, PinX1 may function as a potent telomerase inhibitor and a potential tumor suppressor.

Included in the scope of this invention are PinX1 polypeptide molecules comprising the sequence of SEQ ID NO. 3 or SEQ ID NO. 4 and related biologically active polypeptide fragments and derivatives thereof. The invention also include PinX1-L1 polypeptides comprising the sequence of SEQ ID No. 6 and related biologically active polypeptide fragments and derivatives thereof. In some preferred embodiments, the PinX1 polypeptide comprises at least SEQ ID No. 4.

Further included within the scope of the present invention are polynucleotide molecules (such as SEQ ID No. 1, SEQ ID No. 2, SEQ ID No. 5) that encode the above mentioned polypeptides, and methods for preparing the polypeptides. Such molecules (both polynucleotides and polypeptides) may be useful as therapeutic agents in those cases where modulating telomerase function is desired. Polynucleotides useful according to the invention include the cDNA sequence encoding the full length polypeptide, or the genomic DNA comprising the cDNA sequence, and fragments thereof. In some embodiments of the invention, the PinX1 polynucleotide comprises at least SEQ ID No. 2. Polynucleotides complementary to the above sequences are also within the scope of the present invention.

The DNA containing a nucleotide sequence represented by SEQ ID No. 1, SEQ ID No. 2 SEQ ID No. 5 or an equivalent thereof according to the present invention may be cloned and obtained, for example, by the following techniques:

Gene recombination techniques may be conducted, for example, by the methods disclosed in T. Maniatis et al., “Molecular Cloning”, 2nd Ed., Cold Spring Harbor Laboratory, Cold Spring Harbor, N. T. (1989); Nippon Seikagaku Kai (Biochemical Society of Japan) ed., “Zoku-Seikagaku Jikken Kouza 1, Idenshi Kenkyuho II (Lectures on Biochemical Experiments (Second Series; 1), Methods for Gene Study II)”, Tokyo Kagaku Dojin, Japan (1986); Nippon Seikagaku Kai (Biochemical Society of Japan) ed., “Shin-Seikagaku Jikken Kouza 2, Kakusan III (Kumikae DNA Gijutsu) (New Lectures on Biochemical Experiments 2, Nucleic Acids III (Recombinant DNA Technique))”, Tokyo Kagaku Dojin, Japan (1992); R. Wu (ed.), “Methods in Enzymology”, Vol. 68, Academic Press, New York (1980); R. Wu et al. (ed.), “Methods in Enzymology”, Vols. 100 & 101, Academic Press, New York (1983); R. Wu et al. (ed.), “Methods in Enzymology”, Vols. 153, 154 & 155, Academic Press, New York (1987), etc. as well as by techniques disclosed in the references cited therein, the disclosures of which are hereby incorporated by reference, or by the same techniques as they disclose or modified techniques thereof. Such techniques and means may also be those which are individually modified/improved from conventional techniques depending upon the object of the present invention.

For the above stated uses, the PinX1 or PinX1-L1 polynucleotide is typically cloned into an expression vector, i.e., a vector wherein PinX1 or PinX1-L1 polynucleotides is operably linked to expression control sequences. The need for, and identity of, expression control sequences will vary according to the type of cell in which the PinX1 or PinX1-L1 polynucleotides is to be expressed. Generally, expression control sequences include a transcriptional promoter, enhancer, suitable mRNA ribosomal binding sites, and sequences that terminate transcription and translation. Suitable expression control sequences can be selected by one of ordinary skill in the art. Standard methods can be used by the skilled person to construct expression vectors. See generally, Sambrook et al., 1989, Molecular Cloning: A Laboratory Manual (2nd Edition), Cold Spring Harbor Press, Cold Spring Harbor, N.Y.

Expression vectors are defined herein as DNA sequences that are required for the transcription of cloned copies of genes and the translation of their mRNAs in an appropriate host. Such vectors can be used to express eukaryotic genes in a variety of hosts such as bacteria including E. coli, bluegreen algae, plant cells, insect cells, fungal cells including yeast cells, and animal cells.

A promoter is defined as a DNA sequence that directs RNA polymerase to bind to DNA and initiate RNA synthesis. A strong promoter is one which causes mRNAs to be initiated at high frequency. Expression vectors may include, but are not limited to, cloning vectors, modified cloning vectors, specifically designed plasmids or viruses.

Vectors useful in this invention include plasmid vectors and viral vectors. Preferred viral vectors are those derived from retroviruses, adenovirus, adeno-associated virus, SV40 virus, or herpes viruses.

A variety of mammalian expression vectors may be used to express recombinant PinX1 or PinX1-L1 polynucleotides in mammalian cells. Commercially available mammalian expression vectors which may be suitable for recombinant PinX1 or PinX1-L1 polynucleotide expression, include but are not limited to, pMAMneo (Clontech), pcDNA3 (Invitrogen), pMC1neo (Stratagene), pXT1 (Stratagene), pSG5 (Stratagene), EBO-pSV2-neo (ATCC 37593) pBPV-1(8-2) (ATCC 37110), pdBPV-MMTneo(342-12) (ATCC 37224), pRSVgpt (ATCC 37199), pRSVneo (ATCC 37198), pSV2-dhfr (ATCC 37146), pUCTag (ATCC 37460), 1ZD35 (ATCC 37565), and pEe12 (Celltech).

Recombinant host cells may be prokaryotic or eukaryotic, including but not limited to bacteria such as E. coli, fungal cells such as yeast, insect cells including but not limited to drosophila and silkworm derived cell lines, and mammalian cells and cell lines.

Cell lines derived from mammalian species which may be suitable for expression and which are commercially available, include but are not limited to, CV-1 (ATCC CCL 70), COS-1 (ATCC CRL 1650), COS-7 (ATCC CRL 1651), CHO-K1 (ATCC CCL 61), 3T3 (ATCC CCL 92), NIH/3T3 (ATCC CRL 1658), HeLa (ATCC CCL 2), C127I (ATCC CRL 1616), BS-C-1 (ATCC CCL 26), MRC-5 (ATCC CCL 171), L-cells, HEK-293 (ATCC CRL1573), NS0 (ECACC85110503) and HT1080.

The expression vector may be introduced into host cells via any one of a number of techniques including but not limited to transformation, transfection, protoplast fusion, lipofection, and electroporation. The expression vector-containing cells are clonally propagated and individually analyzed to determine whether they produce PinX1 or PinX1-L1 protein. Identification of PinX1 or PinX1-L1 expressing host cell clones may be done by several means.

In one embodiment, the expression of PinX1 or PinX1-L1 polypeptide is identified using antibodies that are specifically immunoreactive to PinX1 or PinX1-L1 polypeptides. In another embodiment, the expression of PinX1 or PinX1-L1 polypeptide is identified by the presence of host cell-associated PinX1 or PinX1-L1 activity (e.g., binding to Pin2, inhibiting telomerase function).

Examples of the plasmid suitable for host Escherichia coli are pBR322, pUC18, pUC19, pUC118, pUC119, pSP64, pSP65, pTZ-18R/-18U, pTZ-19R/-19U, pGEM-3, pGEM-4, pGEM-3Z, pGEM-4Z, pGEM-5Zf(-), pbluescript KS.TM. (Stratagene) etc. Examples of the plasmid vector suitable for expression in Escherichia coli are pAS, pKK223 (Pharmacia), pMC1403, pMC931, pKC30, etc. The plasmid for host animal cells may include SV40 vector, polyomavirus vector, vaccinia virus vector, retrovirus vector or the like. Examples of the plasmid for host animal cells are pcD, pcD-SRα, CDM8, pCEV4, pME18S, pBC12BI, pSG5 (Stratagene) or the like. Examples of the plasmid for host yeasts are YIp vector, YEp vector, YRp vector, YCp vector, etc., including pGPD-2, etc. Escherichia coli host cells may include those derived from Escherichia coli K12 strains, such as NM533 XL1-Blue, C600, DH1, HB101 and JM109.

Preferably, suitable promoters may be used. For example, such promoters may include tryptophan (trp) promoter, lactose (lac) promoter, tryptophan-lactose (tac) promoter, lipoprotein (1pp) promoter, λ phage P_{L} promoter, etc. in the case of plasmids where Escherichia coli is used as a host; SV40 late promoter, MMTV LTR promoter, RSV LTR promoter, CMV promoter, SRα promoter, etc. in the case of plasmids where an animal cell is used as a host; and GAL1, GAL10 promoters, etc. in the case of plasmids where yeast is used as a host.

Further, the proteins thus obtained can be modified chemically for amino acid residues. The protein can also be modified or partially degraded with enzymes such as pepsin, chymotrypsin, papain, bromelain, endopeptidase, exopeptidase or the like to produce a derivative. In addition, the proteins may be expressed as fusion proteins when they are produced using gene recombinant techniques, which are subjected to in vivo and in vitro conversion into and/or processing to those having a biological activity equivalent to native PinX1 or PinX1-L1. By “a biological activity equivalent to native PinX1 or PinX1-L1, it is meant that a polypeptide comprising a Pin2 or telomerase binding activity. The fusion protein production conventionally used in gene engineering can be employed. Further, such fusion proteins can be isolated and/or purified by means of affinity chromatography or the like wherein the technique employs a fusion portion thereof. The structure of proteins can be modified, improved, etc. by means of methods as described in Nippon Seikagaku Kai (Biochemical Society of Japan) ed., “Shin-Seikagaku Jikken Kouza 1, Tanpakushitsu VII, Tanpakushitsu Kougaku (New Lectures on Biochemical Experiments 1, Proteins VII, (Protein Engineering))”, Tokyo Kagaku Dojin, Japan (1993), the disclosures of which are hereby incorporated by reference, or by techniques as described in references cited therein as well as methods equivalent thereto.

Antibodies Specifically Immunoreactive to PinX1 or PinX1-L1 Polypeptides

One important application of the peptides and proteins of the invention is the generation of antibodies that are specifically to PinX1 or PinX1-L1 polypeptide. The proteins and peptides of the invention can be used to generate antibodies specific for PinX1 or PinX1-L1, or for particular epitopes on those proteins. The PinX1 or PinX1-L1 polypeptide, fragments thereof, or analogs thereof, can be used to immunize an animal for the production of specific antibodies. For the production of antibodies, various hosts, including goats, rabbits, rats, and mice, may be immunized by injection with PinX1 or PinX1-L1 or any portion, fragment or oligopeptide which retains immunogenic properties. Depending on the host species, various adjuvants may be used to increase immunological response. Such adjuvants are commercially available, and include but are not limited to Freund's, mineral gels such as aluminum hydroxide, and surface active substances such as lysolecithin, pluronic polyols, polyanions, peptides, oil emulsions, keyhole limpet hemocyanin, and dinitrophenol. BCG (Bacillus Calmette-Guerin) and Corynebacterium parvum are potentially useful adjuvants.

Monoclonal antibodies to PinX1 or PinX1-L1 can be prepared using any technique which provides for the production of antibody molecules by continuous cell lines in culture. These include but are not limited to the hybridoma technique (originally described by Koehler and Milstein, 1975, Nature 256:495-497, the human B-cell hybridoma technique); (Kosbor et al., 1983, Immunol. Today 4:72; Cote et al., 1983, Proc. Natl. Acad. Sci. 80:2026-2030) and the EBV-hybridoma technique (Cole et al., Monoclonal Antibodies and Cancer Theraphy, Alan R Liss Inc., New York N.Y., pp. 77-96 (1985)). Antibodies may also be produced by inducing in vivo production in the lymphocyte population or by screening recombinant immunoglobulin libraries or panels of highly specific binding reagents as disclosed in Orlandi et al., 1989, Proc. Natl. Acad. Sci. 86: 3833; and Winter and Milstein, 1991, Nature 349:293

Additionally, spleen cells can be harvested from the immunized animal (typically rat or mouse) and fused to myeloma cells to produce a bank of monoclonal antibody-secreting hybridoma cells. The bank of hybridomas can be screened for clones that secrete immunoglobulins that bind the protein of interest specifically, i.e., with an affinity of at least 1×10⁷ M⁻¹. A variety of animals may be used to raise antibodies; for example, mice, rats, goats, rabbits, sheep, and chickens may also be employed to raise antibodies reactive with PinX1 or PinX1-L1. Transgenic animals having the capacity to produce human antibodies also may be immunized and used for a source of antiserum and/or for making monoclonal antibody secreting hybridomas.

Alternatively, or in combination with a recombinantly produced polypeptide, a chemically synthesized peptide having an amino acid sequence corresponding to a PinX1 or PinX1-L1 polypeptide may be used as an immunogen to raise antibodies which bind a PinX1 or PinX1-L1. Immunoglobulins that bind the target protein with a binding affinity of at least about 1×10⁶ M⁻¹ can be harvested from the immunized animal as an antiserum, and may be further purified by immunoaffinity chromatography or other means.

Procedures for raising polyclonal antibodies are also well known. Typically, such antibodies can be raised by administering the protein or polypeptide of the present invention subcutaneously to New Zealand white rabbits which have first been bled to obtain pre-immune serum. The antigens can be injected at six different sites. Each injected material will contain synthetic surfactant adjuvant pluronic polyols, or pulverized acrylamide gel containing the protein or polypeptide after SDS-polyacrylamide gel electrophoresis. The rabbits are then bled two weeks after the first injection and periodically boosted with the same antigen three times every six weeks. A sample of serum is then collected 10 days after each boost. Polyclonal antibodies are then recovered from the serum by affinity chromatography using the corresponding antigen to capture the antibody. Ultimately, the rabbits are euthenized with pentobarbital 150 mg/Kg IV. This and other procedures for raising polyclonal antibodies are disclosed in E. Harlow, et. al., editors, Antibodies: A Laboratory Manual (1988), which is hereby incorporated by reference.

In addition to utilizing whole antibodies, the processes of the present invention encompass use of binding portions of such antibodies. Such binding portions include Fab fragments, F(ab′)₂ fragments, and Fv fragments. These antibody fragments can be made by conventional procedures, such as proteolytic fragmentation procedures, as described in J. Goding, Monoclonal Antibodies: Principles and Practice, pp. 98-118 (N.Y. Academic Press 1983), which is hereby incorporated by reference.

Thus, the invention provides polyclonal and monoclonal antibodies that specifically bind to PinX1 or PinX1-L1. In particular, the present invention also provides antibodies that binds specifically to a polypeptide comprising at least a portion of the amino acid sequence of SEQ ID No. 3 or SEQ ID No. 5. In one embodiment, the present invention provides a pharmaceutical composition comprising at least one antibody, and a pharmaceutically acceptable excipient.

Primers and Probes

The DNA sequences of the present invention are useful for designing primers and probes for isolating and detecting mammal, most preferably human, genomic DNA and cDNA, coding for PinX1 or PinX1-L1 or related proteins thereof. To isolate genes, PCR techniques or PCR using reverse transcriptase (RT) (RT-PCR) can be used. PinX1 or PinX1-L1 cDNA and associated DNA thereof can be used in isolating and detecting PinX1 or PinX1-L1-related genes, via selecting characteristic sequence regions based on amino acid sequences deduced from the cloned and sequenced PinX1 or PinX1-L1 cDNA sequence, then designing and chemically synthesizing DNA primers, and carrying out PCR, RT-PCR, or any other techniques with the obtained DNA primers.

The primers and probes of the present invention may be used for disease diagnosis as described herein.

Antisense DNA

The present invention also provides antisense molecules comprising the nucleic acid sequence complementary to at least 4 consecutive nucleotides (e.g., 10, 20, 50, 100 or more consecutive nucleotides or the full lenth) of the polynucleotide of SEQ ID No. 1 or SEQ ID No. 5. In an alternatively preferred embodiment, the present invention also provides pharmaceutical compositions comprising an antisense molecules complementary in sequence to a sequence of SEQ ID No. 1 or SEQ ID No. 5, and a pharmaceutically acceptable excipient and/or other compound (e.g., adjuvant). Such antisense oligonucleotides have application in reducing transcription of the PinX1 or PinX1-L1 gene and translation of PinX1 or PinX1-L1 mRNA. These antisense oligonucleotides will be administered to patients and cells in which it is desired to reduce the activity or amount of PinX1 or PinX1-L1.

Modulation of PinX1 or PinX1-L1 gene expression can be obtained by using the antisense molecules (DNA, RNA, PNA, and the like) of the invention to target the control regions of the PinX1 or PinX1-L1 gene (i.e., the promoters, enhancers, and introns). Oligonucleotides derived from the transcription initiation site (e.g., between −10 and +10 regions of the mRNA) are often preferred. The antisense molecules may also be designed to block translation of mRNA by preventing the transcript from binding to ribosomes. Similarly, inhibition can be achieved using “triple helix” base-pairing methodology. Triple helix pairing compromises the ability of the double helix to open sufficiently for the binding of polymerases, transcription factors, or regulatory molecules (for a review of recent therapeutic advances using triplex DNA; see Gee et al., in Huber and Carr, Molecular and Immunologic Approaches, Futura Publishing Co, Mt Kisco N.Y. (1994).

Antisense molecules of the invention may be prepared by a wide variety of methods known in the art. These include techniques for chemically synthesizing oligonucleotides, such as solid phase phosphoramidite chemical synthesis. Alternatively, RNA molecules may be generated by in vitro and in vivo transcription of DNA sequences complementary to either strand of the coding sequence of the PinX1 or PinX1-L1 gene. Such DNA sequences may be incorporated into a wide variety of vectors with suitable promoters. Alternatively, antisense cDNA constructs that synthesize antisense RNA constitutively or inducibly can be introduced into cell lines, cells or tissues.

The antisense molecules of the invention may be modified to increase intracellular stability and half-life. Such modifications include, but are not limited to, the addition of flanking sequences at the 5′ and/or 3′ ends of the molecule or the use of phosphorothioate or 2′ O-methyl rather than phosphodiesterase linkages within the backbone of the molecule. The use of PNAs and the inclusion of nontraditional bases such as inosine, queosine and wybutosine as well as acetyl-, methyl-, thio- and similarly modified forms of adenine, cytidine, guanine, thymine, and uridine which are not as easily recognized by endogenous endonucleases can also increase stability.

Detectable Label

In some embodiments of the invention, the primer, the probe, or the antibody specifically immunoreactive to PinX1 or PinX1-L1 polypeptide may be coupled to a detectable label. Various types of detectable labels can be linked to, or incorporated into, a probe, a primer or an antibody of this invention. Examples of useful label types include radioactive, non-radioactive isotopic, fluorescent, chemiluminescent, paramagnetic, enzyme, or colorimetric.

Examples of useful enzyme labels include malate hydrogenase, staphylococcal dehydrogenase, delta-5-steroid isomerase, alcohol dehydrogenase, alpha-glycerol phosphate dehydrogenase, triose phosphate isomerase, peroxidase, alkaline phosphatase, asparaginase, glucose oxidase, β-galactosidase, ribonuclease, urease, catalase, glucose-6-phosphate dehydrogenase, and glucoamylase, acetylcholinesterase. Examples of useful radioisotopic labels include ³H, 131I, 125I, ³²P, ³⁵S, and ¹⁴C. Examples of useful fluorescent labels include fluorescein, rhodamine, phycoerythrin, phycocyanin, allophycocyanin, and fluorescamine. Examples of useful chemiluminescent label types include luminal, isoluminal, aromatic acridinium ester, imidazole, acridinium salt, oxalate ester, luciferin, luciferase, and aequorin.

Suitable labels can be coupled to (e.g., covalently coupled), or incorporated into polynucleotides, polypeptides, antibodies or antibody fragments through standard techniques known to those of ordinary skill in the art. See, for example, Kennedy et al., (1976) Clin. Chim. Acta 70, 1-31; and Schurs et al., (1977) Clin. Chim. Acta 81, 1-40.

The labelling can be accomplished by utilizing the reaction of a thiol group with a maleimide group, reaction of a pyridyldisulfide group with a thiol group, the reaction of an amino group with an aldehyde group, etc. Additionally, it can be selected from widely known methods, methods that can be easily put into practice by an artisan skilled in the art, or any of methods modified therefrom. The coupling agents used for producing the foregoing immunoconjugate or for coupling with carriers are also applicable and usable.

The coupling agents include, for example, glutaraldehyde, hexamethylene diisocyanate, hexamethylene diisothiocyanate, N,N′-polymethylene bisiodoacetamide, N,N′-ethylene bismaleimide, ethylene glycol bissuccinimidyl succinate, bisdiazobenzidine, 1-ethyl-3-(3-dimethylaminopropyl) carbodiimide, succinimidyl 3-(2-pyridyldithio)propionate (SPDP), N-succinimidyl 4-(N-maleimidometyl)cyclohexane-1-carboxylate (SMCC), N-sulfosuccinimidyl 4-(N-maleimidomethyl)-cyclohexane-1-carboxylate, N-succinimidyl (4-iodoacetyl)-aminobenzoate, N-succinimidyl 4-(1-maleimidophenyl)butyrate, N-(.epsilon.-maleimidocaproyloxy)succinimide (EMCS), iminothiolane, S-acetylmercaptosuccinic anhydride, methyl-3-(4′-dithiopyridyl)propionimidate, methyl-4-mercapto-butyrylimidate, methyl-3-mercaptopropionimidate, N-succinimidyl-S-acetylmercaptoacetate, etc.

Diagnosis

Diagnosis of disease associated with telomerase activity may be performed using probes, primers or antibodies of the present invention. The diagnosis, according to the invention, include the detection of a diseased allele (e.g., PinX1 or PinX1-L1) or the detection of the expression of a polynucleotide (e.g., PinX1 or PinX1-L1). A diseased allele may be an insertion, a deletion or substitution of nucleotides in the genomic sequence. The disease include, but are not limited to cancer and aging related disease as described herein.

Diagnosis, according to the invention, include the identification of a disease allele comprising PinX1 or PinX1-L1 polynucleotide, wherein the disease allele comprises a deletion, an insertion, or a point mutation within PinX1 or PinX1-L1 sequence. Diagnosis also refers to any deletion, insertion, or point mutation (e.g., substitution) at polynucleotide sequences flanking PinX or PinX1-L1 polynucleotide, wherein said flanking sequences may affect the expression of PinX1 or PinX1-LS.

Use of Probes

Probes of the present invention may be used to detect disease alleles by any hybridization method well known in the art (e.g., Southern blot, Northern blot, in situ hybridization).

In one embodiment, in situ hybridization is used to quantitate the expression levels of PinX1 or PinX1-L1 mRNA in a mammal. Labeled RNA or DNA that is complementary to a specific mRNA, e.g., PinX1 or PinX1-L1 mRNA, is prepared. Cells or tissue slices are briefly exposed to heat or acid, which fixes the cell contents, including the mRNA, in place on a glass slide, the fixed cell or tissue is then exposed to the labeled complementary RNA for hybridization. Removal of unhybridized labeled RNA and coating the slide with a photographic emulsion is followed by autoradiogaphy to reveal the presence and even the location of specific mRNA within individual cells.

Alternatively, the amount of mRNA in a sample can be measured and quantitated by competition hybridization. In this method, a measured sample of a specific labeled RNA is exposed to just enough complementary DNA to completely hybridize with it, and a sample of unlabeled RNA is then added. If the unlabeled RNA sample contains the same sequence as the labeled RNA, they compete for the DNA, increasing the ratio of unlabeled to labeled samples decreases the amount of labeled RNA hybridized. The extent to which this takes place is a measure of the amount of competing RNA in the unlabeled sample.

The probe may be labeled with a detectable label (e.g., covalently coupled), usually biotin or digoxygenin for in situ hybridization. Following annealing to prepared tissue sections or cells, the label is revealed histochemically, usually using autoradiography (if the label were radioactive), using avidin/streptavidin (if the label were biotin) or using antidigoxygenin antibodies (if the label were digoxygenin).

Genomic DNA, mRNA or cDNA preparation may be used for detection using other hybridization methods (e.g., Southern or Northern blot).

The stringency of the above hybridizations may be changed according to the specific probes used. Methods are well known in the art to detect as low as a single mismatch between the probe and the target sequence in the test sample using high stringent conditions.

Use of Primers

The primers provided by the present invention may be used for detecting a disease allele or the expression of a PinX1 polynucleotide in a mammal. Genomic DNA, mRNA or cDNA preparation may be used.

PCR provides a method for rapidly amplifying a particular polynucleotide sequence by using multiple cycles of DNA replication catalyzed by a thermostable, DNA-dependent DNA polymerase to amplify the target sequence of interest. PCR requires the presence of a nucleic acid to be amplified, two single stranded oligonucleotide primers flanking the sequence to be amplified, a DNA polymerase, deoxyribonucleoside triphosphates, a buffer and salts.

The method of PCR is well known in the art. PCR, is performed as described in Mullis and Faloona, 1987, Methods Enzvmol., 155: 335, herein incorporated by reference.

PCR may be performed using template DNA (at least 1 fg; more usefully, 1-1000 ng) and at least 25 pmol of oligonucleotide primers. A typical reaction mixture includes: 2 μl of DNA, 25 pmol of oligonucleotide primer, 2.5 μl of 10×PCR buffer 1 (Perkin-Elmer, Foster City, Calif.), 0.4 μl of 1.25 μM dNTP, 0.15 μl (or 2.5 units) of Taq DNA polymerase (Perkin Elmer, Foster City, Calif.) and deionized water to a total volume of 25 to 100 μl. Mineral oil may be overlaid and the PCR is performed using a programmable thermal cycler.

The template DNA according to the invention may be genomic extraction or cDNA preparation from a biological sample of a mamnmal.

The length and temperature of each step of a PCR cycle, as well as the number of cycles, are adjusted according to the stringency requirements in effect. Annealing temperature and timing are determined both by the efficiency with which a primer is expected to anneal to a template and the degree of mismatch that is to be tolerated. The ability to optimize the stringency of primer annealing conditions is well within the knowledge of one of moderate skill in the art. An annealing temperature of between 30° C. and 72° C. is used. Initial denaturation of the template molecules normally occurs at between 92° C. and 99° C. for 4 minutes, followed by 20-40 cycles consisting of denaturation (94-99° C. for 15 seconds to 1 minute), annealing (temperature determined as discussed above; 1-2 minutes), and extension (72° C. for 1 minute). The final extension step is generally carried out for 4 minutes at 72° C., and may be followed by an indefinite (0-24 hour) step at 4° C.

Several techniques for detecting PCR products quantitatively without electrophoresis may be useful according to the invention. One of these techniques, for which there are commercially available kits such as Taqman™ (Perkin Elmer, Foster City, Calif.), is performed with a transcriptspecific antisense probe. This probe is specific for the PCR product (e.g. a nucleic acid fragment derived from a gene) and is prepared with a quencher and fluorescent reporter probe complexed to the 5′ end of the oligonucleotide. Different fluorescent markers are attached to different reporters, allowing for measurement of two products in one reaction. When Taq DNA polymerase is activated, it cleaves off the fluorescent reporters of the probe bound to the template by virtue of its 5′-to-3′ exonuclease activity. In the absence of the quenchers, the reporters now fluoresce. The color change in the reporters is proportional to the amount of each specific product and is measured by a fluorometer; therefore, the amount of each color is measured and the PCR product is quantified. The PCR reactions are performed in 96 well plates so that samples derived from many individuals are processed and measured simultaneously. The Taqman™ system has the additional advantage of not requiring gel electrophoresis and allows for quantification when used with a standard curve.

Antibodies

In the present invention, detection and measurement can be carried out by immunostaining including, for example, staining of tissues and cells, immunoassays including, for example, competitive immunoassay and non-competitive immunoassay, radioimmunoassay, ELISA, or the like. Preferably, the detection and measurement is carried out by means of radioimmunoassay, enzyme immunoassay or sandwich assay. In the sandwich-type assay, one of the antibody pair against PinX1 or PinX1-L1 is detectably labeled (e.g., covalently coupled). The other antibody capable of recognizing the same antigen is immobilized on a solid phase. Incubation is carried out to sequentially react a sample to be assayed, labeled antibodies, and immobilized antibodies as required. After the non-binding antibodies are separated, the label or marker is detected or measured. The amount of the measured label is proportional to the amount of antigen, i.e., PinX1 or PinX1-L1. For this assay, simultaneous sandwich assay, forward sandwich assay, or reverse-sandwich assay or the like is called according to the addition sequence of the insolubilized antibody and the labeled antibody. For example, washing, stirring, shaking, filtration, pre-extraction for antigen, etc. is optionally adopted in the measurement process under specific conditions. The other measurement conditions such as specific regents, concentration of buffering solution, temperature or incubation time can vary according to the elements, such as concentration of the antigens in the sample or the nature of samples to be measured. Any person ordinary skilled in the art can suitably select and determine optimal conditions effective for each measurement while using the general experimentation and perform the selected measurement.

Modulating Telomerase Function

Useful modulation, according to the invention, include both increase or decrease telomerase function.

“Enhance” and “increase” are used interchangeable to simply mean to activate or increase a telomerase function in vitro or in vivo by any agent. “Inhibit” or “decrease” are used interchangeable to simply mean to inhibit or decrease a telomerase activity in vitro or in vivo by any agent.

The present invention provides polypeptides which inhibit telomerase function. It's one of the aspect of the present invention to modulate the expression or function of such polypeptide so that the function of telomerase may be modulated to achieve the therapeutic benefits described below.

Inhibiting Telomerase Function

Telomerase activity is detectable in adult somatic cells that have abnormally reactivated the enzyme during the transformation of a normal cell into an immortal tumor cell. Inhibiting telomerase activity provides important benefits to efforts at treating cancer as cancer cells express telomerase activity and normal human somatic cells do not express telomerase activity at biologically relevant levels (i.e. at levels sufficient to maintain telomere length over many cell divisions).

Agents or methods for inhibiting telomerase activity in cancer cells offer therapeutic benefits with respect to a wide variety of cancers and other conditions (for example, fungal infections) in which immortalized cells telomerase activity are a factor in disease progression or in which inhibition of telomerase activity is desired for treatment purposes. In addition, the inhibition of telomerase function in germ line cells may be useful for contraceptive purposes.

Enhancing Telomerase Function

Since the loss of telomeric repeats leads to senescence in somatic cells and is occuring due to the absence of adequate telomerase activity, enhancing telomerase function would have the effect of adding arrays of telomeric repeats to telomeres, thereby imparting to mortal somatic cells increased replicative capacity, and imparting to senescent cells the ability to proliferate and appropriately exit the cell cycle (in the absence of growth factor stimulation with associated appropriate regulation of cell cycle-linked genes typically inappropriately expressed in senescence e.g., collagenase, urokinase, and other secreted proteases and protease inhibitors). Agents and methods for derepressing telomerase in somatic cells may be used transiently or chronically to increase telomere length, and then removed, thereby allowing the somatic cells to again repress the expression of the enzyme utilizing the natural mechanisms of repression.

Enhancing telomerase function would be useful in therapy to forestall and reverse cellular senescence, including but not limited to conditions associated with cellular senescence, e.g., (a) cells with replicative capacity in the central nervous system, including astrocytes, endothelial cells, and fibroblasts which play a role in such age-related diseases as Alzheimer's disease, Parkinson's disease, Huntington's disease, and stroke, (b) cells with finite replicative capacity in the integument, including fibroblasts, sebaceous gland cells, melanocytes, keratinocytes, Langerhan's cells, and hair follicle cells which may play a role in age-related diseases of the integument such as dermal atrophy, elastolysis and skin wrinkling, sebaceous gland hyperplasia, senile lentigo, graying of hair and hair loss, chronic skin ulcers, and age-related impairment of wound healing, (c) cells with finite replicative capacity in the articular cartilage, such as chondrocytes and lacunal and synovial fibroblasts which play a role in degenerative joint disease, (d) cells with finite replicative capacity in the bone, such as osteoblasts, bone marrow stromal fibroblasts, and osteoprogenitor cells which play a role in osteoporosis, (e) cells with finite replicative capacity in the immune system such as B and T lymphocytes, monocytes, neutrophils, eosinophils, basophils, NK cells and their respective progenitors, which may play a role in age-related immune system impairment, (f) cells with a finite replicative capacity in the vascular system including endothelial cells, smooth muscle cells, and adventitial fibroblasts which may play a role in age-related diseases of the vascular system including atherosclerosis, calcification, thrombosis, and aneurysms, and (g) cells with a finite replicative capacity in the eye such as pigmented epithelium and vascular endothelial cells which may play an important role in age-related macular degeneration.

Measuring Telomerase Function

According to the present invention, the modulation (inhibition or activation) of telomerase function may be measured by values of the following five activities: i) telomerase enzymatic activity; ii) telomere length; iii) cell proliferation; iv) cell senescence; and v) cell crisis. Examples are given for each activity, but other methods for measuring all five values are well known in the art and are not limited by the examples listed.

Measuring Telomerase Activity

The enzymatic activity of telomerase can be measured as described herein, or by any other existing methods or equivalent methods well known in the art. By “increase” of such activity is meant that the absolute level of telomerase activity in the particular cell is elevated compared to normal cells in that individual, or compared to normal cells in other individuals not suffering from the condition. Examples of such conditions include cancerous conditions, or conditions associated with the presence of cells which are not normally present in that individual, such as protozoan parasites or opportunistic pathogens, which require telomerase activity for their continued replication.

Human telomerase activity may be determined by measuring the rate of elongation of an appropriate repetitive sequence (primer), having 2 or more, usually 3 or more, repeats of the telomere unit sequence, TTAGGG (SEQ ID No. 15, see Yegorov, Y. E., Chemov, D. N., Akimov, S. S., Bolsheva, N. L., Kraevsky, A. A., and Zelenin, A. V. (1997) Mol. Biol. (Moscow), 31, 130-136). The sequence is labeled with a specific binding pair member at a convenient site, e.g., the 5′-terminus, and the specific binding pair member allows for separation of extended sequences. By using one or more radioactive nucleoside triphosphates or other labeled nucleoside triphosphate, as described previously, one can measure the incorporated radioactivity as cpm per unit weight of DNA as a function of unit of time, as a measure of telomerase activity. Any other detectable signal and label may also be used, e.g., fluorescein.

The activity may be measured with cytoplasmic extracts, nuclear extracts, lysed cells, whole cells, and the like (e.g., Morin, G. B. (1989) Cell, 59, 521-529). The particular sample which is employed and the manner of pretreatment will be primarily one of convenience. The pretreatment will be carried out under conditions which avoids denaturation of the telomerase, so as to maintain the telomerase activity. The primer sequence will be selected or labeled so as to allow it to be separated from any other DNA present in the sample. Thus, a haptenic label may be used to allow ready separation of the elongated sequence, which represents the telomerase activity of the sample. The nucleoside triphosphates which may be employed may include at least one nucleoside triphosphate which is labeled. The label will usually be radiolabel, but other labels may also be present. The labels may include specific binding pair members, where the reciprocal member may be labeled with fluorescent, enzymes, or other detectable label. Alternatively, the nucleoside triphosphates may be directly labeled with other labels, such as fluorescent labels.

The sequence elongation usually will be carried out at a convenient temperature, generally from about 20-40° C. and for a time sufficient to allow for at least about 100 bp to be added on the average to the initial sequence, generally about 30-90 minutes. After the incubation time to allow for the telomerase catalyzed elongation, the reaction may be terminated by any convenient means, such as denaturation, e.g., heating, addition of an inhibitor, rapid removal of the sequence by means of the label, and washing, or the like. The separated DNA may then be washed to remove any non-specific binding DNA, followed by a measurement of the label by any conventional means.

Other techniques for measuring telomerase activity can use antibodies specific for the telomerase protein, where one may determine the amount of telomerase protein in a variety of ways. For example, one may use polyclonal antisera bound to a surface of monoclonal antibody for a first epitope bound to a surface and labeled polyclonal antisera or labeled monoclonal antibody to a second epitope dispersed in a medium, where one can detect the amount of label bound to the surface as a result of the telomerase or subunit thereof bridging between the two antibodies. Alternatively, one may provide for primers to the telomerase RNA and using reverse transcriptase and the polymerase chain reaction, determine the presence and amount of the telomerase RNA as indicative of the amount of telomerase present in the cells.

Measuring Telomere Length

Procedures for measuring telomere length are known in the art and can be used in this invention (e.g., Harley, C. B., Futcher, A. B., and Greider, C. W. (1990) Nature, 345, 458-460. Levy, M. Z., Allsopp, R. C., Futcher, A. B., Grieder, C. W., and Harley, C. B. (1992) J. Mol. Biol., 225, 951-960; Lindsey, J., McGill, N. I., Lindsey, L. A., Green, D. K., and Cooke, H. J. (1991) Mutat. Res., 256, 45-48; Allsopp, R. C., Vaziri, H., Patterson, C., Goldstein, S., Younglai, E. V., Futcher, A. B., Greider, C. W., and Harley, C. B. (1992) Proc. Natl. Acad. Sci. USA, 89, 10114-10118). Typically, restriction endonuclease digestion is used (with enzymes which do not cleave telomeric DNA), and the length of the fragment having detectable telomere DNA is separated according to molecular weight by agarose gel electrophoresis. Given that the DNA sequence of a telomere is known, detection of such DNA is relatively easy by use of specific oligonucleotides. Examples of these methods are provided below.

In detection of the telomeric length, one may study just a particular cell type, all cells in a tissue (where various cells may be present), or subsets of cell types, and the like. The preparation of the DNA having such telomeres may be varied, depending upon how the telomeric length is to be determined.

Conveniently, the DNA may be isolated in accordance with any conventional manner, freeing the DNA of proteins by extraction, followed by precipitation. Whole genomic DNA may then be melted by heating to at least about 80° C., usually at least about 94° C., or using high salt content with chaotropic ions, such as 6×SSC, quanidinium thiocyanate, urea, and the like. Depending upon the nature of the melting process, the medium may then be changed to a medium which allows for DNA synthesis.

(a) DNA Synthesis

In one method, a primer is used having at least about 2 repeats, preferably at least about 3 repeats of the telomeric sequence, generally not more than about 8 repeats, conveniently not more than about 6 repeats. The primer is added to the genomic DNA in the presence of only 3 of the 4 nucleoside triphosphates (having the complementary nucleosides to the protruding or G-rich strand of a telomere, e.g., A, T and C for human chromosomes), DATP, dTTP and dCTP. Usually at least the primer or at least one of the triphosphates is labeled with a detectable label, e.g., a radioisotope, which label is retained upon incorporation in the chain. If no label is used, other methods can be used to detect DNA synthesis. The primer is extended by means of a DNA polymerase, e.g., the Klenow fragment of DNA polymerase I, T7 DNA polymerase or Taq DNA polymerase.

The length of the extended DNA can then be determined by various techniques, e.g., those which separate synthesized DNA on the basis of its molecular weight, e.g., gel electrophoresis. The DNA synthesized may then be detected based on the label, e.g., counts incorporated per μg of DNA, where the counts will be directly proportional to telomere length. Thus, the measure of radioactivity in relation to the amount of DNA will suffice to quantitate telomere length.

If desired, telomeres of known length may be used as standards, whereby a determination of radioactivity may be read of f a standard curve as related to telomere length. Instead, one may prepare tissues where individual cells may be assayed for relative telomere length by in situ hybridization. In this approach, for example, the primer is labeled with a detectable label, usually biotin or digoxygenin. Following annealing to prepared tissue sections or cells, the label is revealed histochemically, usually using autoradiography (if the label were radioactive), using avidin/streptavidin (if the label were biotin) or using antidigoxygenin antibodies (if the label were digoxygenin). The amount of signal per cell is proportional to the number of telomeric repeats, and thus to the telomere length. This can be quantitated by microfluorometry or analogous means, and compared to the signal from standard cells of known telomere length to determine the telomere length in the test sample.

(b) Restriction Endonuclease Digestion

Alternatively, one may use primers which cause covalent cross-linking of the primer to telomere DNA. In this situation, one may totally digest the DNA with restriction endonucleases which have 4 base recognition sites, which results in the production of relatively short fragments of DNA, except for telomeric DNA which lacks the recognition site. Restriction endonucleases which may find use include AluI, HinfI, MspI, RsaI, and Sau3A, where the restriction endonucleases may be used individually or in combination. After digestion of the genomic DNA, the primer may be added under hybridizing conditions, so as to bind to the protruding chain of the telomeric sequence. By providing for two moieties bound to the primer, one for covalent bonding to the telomeric sequence and the other for complex formation with a specific binding pair member, one can then provide for linking of a telomeric sequence to a surface. For example, for covalent bonding to the telomeric sequence, psoralen, or isopsoralen, may be linked to one of the nucleotides by a bond or chain and upon UV-radiation, will form a bridge between the primer and the telomere.

The specific binding pair member will normally be a hapten, which binds to an appropriate complementary member, e.g., biotin and strept/avidin, trinitrobenzoic acid and anti-trinitrobenzamide antibody, or methotrexate and dihydrofolate reductase. Rather than having the moiety for covalent bonding covalently bonded to the primer, one may add a compound into the medium which is intercalatable into the nucleic acid, so as to intercalate between double-stranded nucleic acid sequences. In this manner, one may achieve the same purpose. Use of a substantial excess of the intercalatable compound will cause it to also intercalate into other portions of DNA which are present. Various modifications of this process may be achieved, such as size separation, to reduce the amount of label containing DNA.

The specific binding pair member may be used for separation of telomeric DNA free of contaminating DNA by binding to the complementary pair member, which may be present on beads, on particles in a column, or the like. In accordance with the nature of the separation, the covalently bonded telomere strand may now be purified and measured for size or molecular weight. Again, if desired, standards may be employed for comparison of distribution values.

The specific binding pair member hapten can be present at the 5′-terminus of the primer or at intermediate nucleotides. Specifically, biotin-conjugated nucleotides are generally available and may be readily introduced into synthetic primer sequences in accordance with known ways.

The above-described techniques can also be used for isolating and identifying DNA contiguous to the telomere.

(c) Average Telomere Length

In methods of this invention it may be useful to determine average telomere length by binding a primer to a telomere prior to separation of the telomeric portion of the chromosomes from other parts of the chromosomes. This provides a double-stranded telomeric DNA comprising the telomeric overhang and the primer. A reaction may then be carried out which allows for specific identification of the telomeric DNA, as compared to the other DNA present. The reaction may involve extension of the primer with only 3 of the nucleotides (dNTPs), using a labeled nucleotide, covalent bonding of the primer to the telomeric sequence, or other methods which allow for separation of the telomeric sequence from other sequences. The length of the synthesized DNA detected then represents the average telomere length.

Telomere length can also be measured directly by the “anchored terminal primer” method. In this method, the 3′ ends of genomic DNA are first “tailed” with dG nucleotides using terminal transferase. Telomeres, which are known to have 3′ overhangs, then would have one of the three following conformations:

| | |
|---|---|
| 5′TTAGGGTTAGGGTTAGGGGGGGGGGG...3′ | (SEQ ID No. 9) |
| 5′TTAGGGTTAGGGTTGGGGGGGGGGGG...3′ | (SEQ ID No. 10) |
| 5′TTAGGGTTAGGGTGGGGGGGGGGGGG...3′ | (SEQ ID No. 11) |

Other ends of the genomic DNA which were generated by shearing would be tailed with G's but would not have the adjacent TTAGGG repeats. Thus, a mix of the following 3 biotinylated oligonucleotides would anneal under stringent conditions specifically to all possible telomere ends:

| | |
|---|---|
| 5′B-CCCCCCCCTAACCCTA | (SEQ ID No. 12) |
| 5′B-CCCCCCCCAACCCTAA | (SEQ ID No. 13) Oligo Mix [M] |
| 5′B-CCCCCCCCACCCTAAC | (SEQ ID No. 14) |

Oligo mix [M] consists of 16-base oligonucleotides with 5′ biotin (B), but other combinations of 5′-C-tracts adjacent to the C-rich telomeric repeats could provide specific hybridization to the 3′ end of the native telomeres.

Extension of the primer with a DNA polymerase such as Klenow, DNA Polymerase I, or Tag polymerase, in the presence of dCTP, DATP, dTTP (no dGTP, and with or without ddGTP) would stabilize the primer-template configuration and allow selection, using streptavadin beads, of the terminal fragments of DNA containing the telomeric DNA. The length of primer extension using Klenow (monitored with labeled nucleotides) would indicate the length of the telomeric (GTR) 3′ overhang, since Klenow lacks 5′-3′ exonuclease activity and would stall at the CTR. This length distribution could be indicative of the level of telomerase activity in telomerase-positive cells (i.e., longer extensions correspond to greater telomerase activity). In contrast, extension of the primer with DNA polymerase I, an enzyme with 5′-3′ exonuclease activity as well as polymerase activity, would allow extension through the CTR until C's are encountered in the template strand (subtelomeric to the GTR). The length distribution of this reaction, monitored by labeled nucleotides, would be indicative of the length distribution of the GTR. In both cases, labeled products arising from biotinylated primers are selected with the streptavadin beads to reduce the signal from non-specific priming. Alternatively, re-priming and extension of the tailed chromosome end can take place after selection of the partially extended products with the streptavadin beads, and after denaturation of the C-rich strand from the duplex.

Experiments have confirmed that the G-tailing of chromosome ends can be carried out efficiently such that about 50 G residues are added per end, that the priming with the junction oligonucleotide mix is highly specific for the tailed telomeric ends, and that streptavadin beads select specifically for the extension products that originate from the biotinylated primers and not from other fortuitous priming events. The length of the extension products under the conditions outlined above thus provide a direct estimate of the length of the terminal TTAGGG (SEQ ID No. 15) repeat tract. This information is especially important in cases where stretches of TTAGGG (SEQ ID No. 15) repeats occur close to but not at the termini of chromosomes. No other method described to date is capable of distinguishing between the truly terminal TTAGGG (SEQ ID No. 15) repeats and such internal repeats.

It is possible to determine the amount of telomeric DNA on individual chromosomes by FISH using fluorescently labeled oligo- or polynucleotide probes. Chromosomes can be collected from metaphase cells, wherein they are identified by shape and/or banding patterns using staining procedures or secondary probes of a different fluorescent color, or they can be spread and stretched from interphase cells. In the later case, it is possible again to identify specific chromosomes with fluorescently labeled secondary probes complementary to sequences close to the telomere. Quantitative FISH with confocal microscopy or imaging systems using signal integration or contour length allows one to obtain an objective measure of the distribution of telomere lengths on different chromosomes and to identify chromosomes which have potentially lost a critical amount of telomeric DNA.

d) Modified Maxam-Gilbert Reaction

The most common technique currently used to measure telomere length is to digest the genomic DNA with a restriction enzyme with a four-base recognition sequence like HinfI, electrophorese the DNA and perform a Southern blot hybridizing the DNA to a radiolabeled (TTAGGG)₃ (SEQ ID No. 16) probe. A difficulty with this technique is that the resulting terminal restriction fragments (TRFs) contain a 3-5 kb stretch of subtelomeric DNA that lacks restriction sites and thereby adds significantly to the size of the measured telomere length. Another approach to eliminate this DNA and improve accuracy of telomere length assays utilizes the fact that this subtelomeric DNA contains G and C residues in both strands, and thus should be cleaved under conditions that cause breaks at G residues. In contrast, DNA composed exclusively of telomeric repeats will have one strand lacking G residues, and this strand should remain intact under G-cleavage conditions. The Maxam-Gilbert G-reaction uses piperidine to cleave guanine residies that have been methylated by dimethylsulfate (DMS) treatment. Although the original conditions of the Maxam-Gilbert G-reaction (treatment in 1M piperidine for 30 min at 90° C.) breaks unmethylated DNA into fragments of 1-2 kb and is thus non-specific, milder conditions (0.1 M piperidine for 30 min at 37° C.) leave untreated DNA intact. The DNA is therefore treated with DMS and piperidine as described above, precipitated with ethanol, electrophoresed, and hybridized on a Southern blot to the a (TTAGGG)₃ (SEQ ID No. 16) probe.

Cell Proliferation

Proliferation measurement may be carried out by methods well known in the art.

Cell proliferation rate can be measured by cell doubling time as described in, for example, Harley, C. B., Futcher, A. B., and Greider, C. W. (1990) Nature, 345, 458-460.

For the quantification of cell proliferation, a ³H incorporation assay or a calorimetric ELISA BrdU incorporation assay (Boehringer Mannheim) may be used.

Cell Senescence and Crisis

As cells progress to a senescent state, the cells exhibit an elongation of the G1 phase of the cell cycle, leading to a longer cell time of cycle transit (Hayflick, L. and Moorhead, P. S., Exp. Cell Res. 25:585-621 (1961); Hayflick, L., Exp. Cell Res. 37:614-636 (1965. As the progression from a mitotically active to a senescent state continues, cells fail to respond to mitotic signals and remain in G1. This inability of senescent cells to enter the cell cycle represents a significant difference between young and old cells. Unlike old cells, young cells become quiescent entering G0 but can be subsequently induced to reenter the cell cycle and divide. However, senescent cells, while remaining viable and metabolically active, become refractile to entering the cell cycle.

Cells arrest in the G1 phase of the cell cycle and contain a 2N chromosomal complement (Cristofalo, V. J., et al., Exp. Gerontol. 24:367 (1989)). This in phase, or clonal, senescence of the HDFs is accompanied by a characteristic morphological change; cells enlarge as they senesce (Angello, J. C., et al., J. Cell. Physiol. 132:125-130 (1987) and Cristofalo, V. J. and Kritchevsky, D., Med. Exp. 19:313-320 (1969). In fact, this direct correlation between cell size and senescence can be demonstrated by incubating young HDFs in low serum-medium, in which they enlarge, but do not leave the G1 phase of the cell cycle (Angello, J. C., et al., J. Cell. Physiol. 140:28-294 (1989). When these cells are returned to medium containing adequate serum for cell division, their program of senescence has been advanced compared to smaller cells which have divided the same number of times.

A characteristic of replicative senescence is that changes in the pattern of gene expression can be observed as the cell progresses through its replicative lifespan. These changes are reflected in a decrease in the expression of “young-specific” genes and an increase in the expression of “old-specific” genes. Together, these young- and old-specific genes are referred to herein as “senescence-associated” genes, where a senescence-associated gene is any gene for which the product of the gene is differentially expressed between young quiescent cells and senescent cells. Not only do these changes affect the structure and function of the senescent cell, but also such changes can influence the physiology of surrounding cells and the tissue matrix by altering the extracellular environment, i.e., in a paracrine fashion through the release of different proteins or through changes in cell-cell interactions. Several senescence-specific genes have been described (Linskens et al., PCT No. WO 96/13610, published May 9, 1996 and incorporated herein by reference).

Examples of “senescence-associated” genes and markers include: β-galactosidase, collagenase, interferon gamma, collagen I, collagen III, elastase, elastin, TIMP3, or IL-Ia, autofluorescence, acridine-orange fluorescence, and telomere length

The above senescence associated genes and markers, cell-arrest, and cell morphology can be examined by methods well known in the art.

Cells when grown to crisis, wherein the M2 mechanism is preventing their growth may be detected by using one of the crisis markers such as cell morphology, cell cycle profile, reduction of cell proliferation, etc. by methods well known in the art. For example, cell proliferation may be measured by BrdU incorporation assay as described herein.

Screening Assays

The invention can be used to screen candidate agents for the ability to i) bind to Pin2 protein; ii) modulate the interaction between Pin2 and PinX1; iii) modulate PinX1 or PinX1-L1 expression.

In some embodiments, the two-hybrid expression system described below is used to screen for the above candidate agents in vivo. The two-hybrid method is a well known yeast-based genetic assay to detect protein-protein interactions in vivo (See, e.g., Bartel et al., 1993, In Cellular Interactions in Development: A Practical Approach, Oxford University Press, Oxford, pp. 153-179; Chien et al., 1991, Proc. Natl. Acad. Sci. USA, 88:9578-9582; Fields et al., 1989, Nature, 340:245-247; Fritz et al., 1992, Curr. Biol., 2:403-405; Guarente, L., 1993, Proc. Natl. Acad. Sci. USA, 90:1639-1641).

In one embodiment for screening agents bind to Pin2, a GAL4 binding site, linked to a reporter gene such as lacZ, is contacted with a GAL4 binding domain linked to a Pin2 polynucleotide and a GAL4 transactivation domain II linked to a cDNA expression library. Expression of the reporter gene is monitored, and the presence of expression from such reporter gene indicates a candidate Pin2 binding polypeptide encoded by such cDNA library.

The preparation of cDNA is well-known and well-documented in the art.

mRNA samples can be isolated from various human tissues (placenta, oral tumor, lung cancer, etc.), culture cells (human fibrosarcoma HT1080 cell line, human monocytic leukemia U937 cell line, etc.) and the like. In particular, mRNA can preferably be isolated from a human oral tumor cell (oral malignant melanoma). Although, in an embodiment, mRNA may be isolated with a method known in the art or by the same method as it is or modifications thereof, the isolation and purification of mRNA can be conducted by methods disclosed in, for example, T. Maniatis, et al., “Molecular Cloning”, 2nd Ed., Chapter 7, Cold Spring Harbor Laboratory, Cold Spring Harbor, N. T. (1989); L. Grossman, et al. ed., “Methods in Enzymology”, Vol. 12, Parts A & B, Academic Press, New York (1968); S. L. Berger et al. ed., “Methods in Enzymology”, Vol. 152, p. 33 & p. 215, Academic Press, New York (1987); Biochemistry, 18, 5294-5299, 1979; etc., the disclosures of which are hereby incorporated by reference. Examples of such mRNA isolating and purifying techniques are a guanidine-cesium chloride method, a guanidine thiocyanate method, a phenol method, etc. If necessary, the resulting total RNA may be subjected to a purification process using an oligo(dT)-cellulose column, etc. to give poly(A).+sup. mRNA.

cDNAs are prepared by using, as a template, the resulting mRNA and a reverse transcriptase, etc. The reverse transcriptase synthesis of cDNA using mRNA may be carried out by standard techniques known in the art, by the same techniques or by modified techniques thereof. Detailed techniques are found in, for example, H. Land et al., “Nucleic Acids Res.”, Vol. 9, 2251 (1981); U. Gubler et al., “Gene”, Vol. 25, 263-269 (1983); S. L. Berger et al. ed., “Methods in Enzymology”, Vol. 152, p. 307, Academic Press, New York (1987); etc., the disclosures of which are hereby incorporated by reference.

Then, based upon the cDNA thus prepared, cDNA libraries can be constructed. Besides the technique using a phage vector, transformations of host cells including Escherichia coli may be conducted according to techniques known in the art, such as a calcium technique and a rubidium/calcium technique, or the same methods (D. Hanahan, J. Mol. Biol., Vol. 166, p. 557 (1983), etc.). Various commercially available cDNA libraries derived from human tissues (for example, obtainable by CLONTECH, etc.) can also be used directly. A polymerase chain reaction (PCR) is conducted using the prepared cDNA as a template. In an embodiment, primers are synthesized which have degenerate oligonucleotides designed from highly conserved regions selected from amino acid sequences identical between PinX1 and PinX1-L1. Preparation of primers may be carried out by techniques which are known in the art. For example, the primers may be synthesized by means of a phosphodiester method, a phosphotriester method, a phosphoamidite method, etc. using an automatic DNA synthesizer. The PCR amplification is carried out using said primers and the template cDNA thus prepared. The PCR may be carried out by techniques known in the art or by methods equivalent thereto or modified techniques. The reaction may be conducted by the methods disclosed, for example, in R. Saiki, et al., Science, Vol. 230, pp. 1350 (1985); R. Saiki, et al., Science, Vol. 239, pp. 487 (1985); and PCR Technology, Stockton Press; etc., the disclosures of which are hereby incorporated by reference.

The resulting PCR products are cloned, and sequenced. Sequencing of nucleotide sequences may be carried out by a dideoxy technique (such as an M13 dideoxy method), a Maxam-Gilbert method, etc. or may be carried out using a commercially available sequencing kit such as a Taq dyeprimer cycle sequencing kit or an automated nucleotide sequencer such as a fluorescent DNA sequencer.

In another ambodiment for screening agents bind to Pin2, the Pin2 polypeptide is immobilized. The immobilized Pin2 protein is then contacted with a protein extract to allow a candidate polypeptide in the protein extract to form a complex with the immobilized Pin2. Unbound protein can be removed by washing. The complex then can be solubilized and analyzed to determine the identity and amount of bound candidate polypeptide.

Polypeptides can be immobilized using methods known in the art. Such methods include adsorption onto a plastic microtiter plate or specific binding of a glutathione-S-transferase (GST)-fusion protein to a polymeric bead containing glutathione.

In one embodiment for screening candidate agent modulating the binding between Pin2 and PinX1, a GAL4 binding site, linked to a reporter gene such as lacZ, is contacted with a GAL4 binding domain linked to a Pin2 polynucleotide and a GAL4 transactivation domain II linked to a PinX1 polypeptide comprising the c-terminal domain in the presence and absence of a candidate agent. Expression of the reporter gene is monitored, and a decrease or an increase of expression from such reporter gene indicates a candidate agent modulating the binding between Pin2 and PinX1.

In one embodiment for screening candidate agent modulating the binding between Pin2 and PinX1, one of the protein is immobilized. The immobilized protein (e.g., Pin2 ) is then contacted with a labeled protein to which it binds (PinX1 in this example) in the presence and absence of a candidate agent. Unbound protein can be removed by washing. The complex then can be solubilized and analyzed to determine the amount of bound (labeled) protein. A decrease or an increase in binding is an indication that the candidate agent modulates binding between Pin2 and PinX1.

Another important use of the oligonucleotide and antibody probes of the present invention is in a method for screening compounds to identify compounds that can alter PinX1 or PinX1-L1 gene expression, which method comprises: (a) contacting said cells with an agent; (b) measuring an amount of a PinX1 or PinX1-L1 gene product of said treated cells; (c) comparing said measured amount of said PinX1 or PinX1-L1 gene product with a measured amount of said PinX1 or PinX1-L1 gene product of a control cell not contacted with said agent; and (d) identifying as agents that alter senescent gene expression in cells as any agent that produces an increased or decreased amount of said PinX1 or PinX1-L1 gene product in said treated cells in relative to said control cells.

Any PinX1 or PinX1-L1 gene product can be used in the method; for example, PinX1 or PinX1-L1 mRNA or polypeptide.

This screening method identifies agents with the capacity to reverse, partially reverse, inhibit, or enhance PinX1 or PinX1-L1 gene expression. The present invention also encompasses the compounds identified by this method and the use of those agents to alter telomerase function in disease cells.

In general, the basic format of the screen is as follows. Cells are cultured in 96-well microtiter plates. After an incubation period, i.e., three days in culture, the medium will be removed and the cells can optionally be assayed for PinX1 or PinX1-L1 gene products, providing a “before treatment” baseline, if desired. The medium will be replaced with fresh medium containing a test agent or its vehicle. The cells will be cultured for an additional period, i.e., two to four days or more in culture, in the presence of the test agent. The cells and/or medium will then be assayed for PinX1 or PinX1-L1 gene products (“after treatment” measurement) and compared to non-treated controls.

Cell-based screens have traditionally been labor intensive and so have not often been used for high-throughput screening. However, the present method is amenable to high-throughput screening. Liquid handling operations can be performed by a Microlab 2000™ pipetting station (Hamilton Instruments). Other equipment needed for the screen (e.g., incubators, plate washers, plate readers) can either be adapted for automated functioning or purchased as automated modules. Movement of samples through the assay can be performed by an XPTM robot mounted on a 3 m-long track (Zymark).

In addition, PinX1 or PinX1-L1 or its c-terminal fragments or oligopeptides thereof can be used for screening therapeutic compounds in any of a variety of other drug screening techniques. In particular, the PinX1 or PinX1-L1 gene product is a useful target for therapeutic intervention because that gene product may be involved in disease pathology and a change in its expression parallels that of gene products involved in disease pathology. One can quantitate changes in the level of gene expression caused by a agent using high-throughput screening techniques. The PinX1 or PinX1-L1 gene product or fragment thereof employed in such a test may be free in solution, affixed to a solid support, born on a cell surface, or located intracellularly. The formation of binding complexes, between the gene product and the agent being tested, may be measured.

Through these screens, libraries of synthetic organic compounds, natural products, peptides, and oligonucleotides can be evaluated for their capacity to alter i) binding between Pin2 and PinX1 or PinX1-L1; ii) PinX1 or PinX1-L1 expression that may be useful in disease treatment. Active agents can be optimized, if desired, via medicinal chemistry. Initially, one can define a pharmacophore(s) using modem computational chemistry tools representative of the structures found to be active in the high throughput screens. Once a consensus pharmacophore is identified, one can design focused combinatorial libraries of agents to probe structure-activity relationships. Finally, one can improve the biopharmaceutical properties, such as potency and efficacy, of a set of lead structures to identify suitable agents for clinical testing.

Therapies

The polynucleotides, proteins, antisense DNAs, antibodies, and PinX1 or PinX1-L1 agonists, antagonists, or inhibitors, are employed to treat disease related to telomerase function.

Because telomerase is active only in cancer, germline, and certain stem cells of the hematopoietic system, other normal cells are not affected by telomerase inhibition therapy. Steps also can be taken to avoid contact of telomerase inhibitor with germline or stem cells, although this may not be essential. For instance, because germline cells express telomerase activity, inhibition telomerase may negatively impact spermatogenesis and sperm viability, suggesting that telomerase inhibitors may be effective contraceptives or sterilization agents. This contraceptive effect may not be desired, however, by a patient receiving a telomerase inhibitor of the invention for treatment of cancer. In such cases, one can deliver a telomerase inhibitor of the invention in a manner that ensures the inhibitor will only be produced during the period of therapy, such that the negative impact on germline cells is only transient.

Gene Therapy

Once a therapeutic gene is defined, the gene sequence is subcloned into a vector suitable for the purpose of gene therapy. Murine leukemia virus (MLV)-based retroviral vectors are one of the most widely used gene delivery vehicles in gene therapy clinical trials and have been employed in almost 70% of approved protocols (Ali, M. et al., Gene Ther., 1:367-384, 1994; Marshall, E., Science, 269:1050-1055, 1995). Other useful vectors are also known in the art (e.g., Carter and Samulski, 2000, Int. J. Mol. Med. 6:17-27; Lever et al., 1999, Biochem. Soc. Trans. 27: 841-7. Methods for gene therapy of human diseases are described in U.S. Pat. Nos. 6,190,907; 6,187,305; 6,140,087; and 6,129,705.

Transfection of cells, whether in vitro, ex vivo or in vivo, involves not only delivery of the transfecting DNA to the cell nucleus, but also expression of the delivered DNA in the cell. Some gene delivery systems involve transfection of cells using a delivery complex in which DNA is condensed with cationic polymers such as cationic lipids or polylysine (see, for example, Cotten and Wagner (1993) Curr. Opin. Biotech., 4: 705).

One promising strategy for agent delivery involves somatic gene therapy. Cells in a desired region of the body are engineered to express a gene corresponding to a therapeutically or diagnostically useful protein. Genetic information necessary to encode and express the protein is transferred to the cells by any of a number-of techniques, including viral vectors, electroporation, receptor-mediated uptake, liposome masking, precipitation, incubation and others. Gene therapy can be a direct in vivo process where genetic material is transferred to cells in the desired region of the patient's body. Most current in vivo strategies rely on viral vectors. Alternatively, the process can be an indirect in vitro process where cells from the desired region are harvested, genetic material is transferred to the cells, and the cells are implanted back in the patient's body. In vitro techniques allow for more flexibility in transfer methods and may be safer since viral vectors need not be introduced into the patient's body, thus avoiding the theoretical risk of insertional mutations, replication reactivation and other harmful consequences.

One region of interest for gene therapy is the circulatory system. Researchers have transferred genetic material to the vascular walls, particularly the smooth muscle and endothelial cells. Suitable delivery techniques include ligation of the vessel (Lynch et al., supra.), dual-balloon catheters (Leclerc G et al., J. Clin. Invest. 90:936-44 (1992)), perforated balloon catheters (Flugelman M Y et al., Circulation 85:1110-17 (1992)); stents seeded with transduced endothelial cells (Dichek D A et al., Circulation 80:1347-53 (1989)) and vascular grafts lined with transduced endothelial cells (Wilson J M et al., Science 244:1344-46 (1989).

Polypeptide and Antibody Therapy

PinX1 or PinX1-L1 polypeptides and antibodies can be administered in many possible formulations, including pharmaceutically acceptable media. In the case of a short peptide, the peptide can be conjugated to a carrier, such as KLH, in order to increase its ability to cause an effect on the immune system. The composition can include or be administered in conjunction with an adjuvant, of which several are known to those skilled in the art. After initial immunization with the vaccine, further boosters can be provided. The compositions are administered by conventional methods, in dosages which are sufficient to cause an effect on the immune system. Such dosages can be easily determined by those skilled in the art.

U.S. Pat. No. 6,043,339 provides a method of importing a biologically active molecule into a cell ex vivo comprising administering to the cell, under import conditions, a complex comprising the molecule linked to an importation competent signal peptide, thereby importing the molecule into the cell. Molecules that can be delivered by this method can include, for example, peptides, polypeptides, proteins, nucleic acids, carbohydrates, lipids, glycolipids, and therapeutic agents.

U.S. Pat. No. 6,187,330 provides a composition for the controlled release of a peptide or protein comprising a biocompatible, bioerodable polymer having dispersed therein a glassy matrix phase comprising the peptide or protein and a thermoprotectant, said glassy matrix phase having a glass transition temperature above the melting point of the polymer. Since the peptide or protein drug is stable within the composition, it can conveniently be formed, in its melt stage, into suitably shaped devices to be used as drug delivery implants, e.g. in the form of rods, films, beads or other desired shapes.

Pharmaceutical Formulations and Administration

The invention further comprises the therapeutic prevention/treatment of cancer or aging through telomerase modulation by the administration of an effective dose of i) PinX1 or PinX1-L1 polynucleotide or fragments thereof; ii) PinX1 or PinX1-L1 polypeptide or fragments thereof; iii) an antibody specifically immunoreactive to PinX1 or Pin X1 polypeptides; or iv) an antisense polynucleotide complementary to PinX1 or PinX1-L1 polynucleotide. Where clinical applications are contemplated, it will be necessary to prepare pharmaceutical compositions of drugs in a form appropriate for the intended application. Generally, this will entail preparing compositions that are essentially free of pyrogens, as well as other impurities that could be harmful to humans or animals.

The use of pharmaceutically acceptable carrier for pharmaceutically active agents is well know in the art. Supplementary active ingredients also can be incorporated into the compositions.

The active compositions of the present invention may include classic pharmaceutical preparations. Administration of these compositions according to the present invention will be via any common route so long as the target tissue is available via that route. This includes oral, nasal, buccal, rectal, vaginal or topical. Alternatively, administration may be by orthotopic, intradermal, subcutaneous, intramuscular, intraperitoneal or intravenous injection. Such compositions would normally be administered as pharmaceutically acceptable compositions, described supra. A preferred route is direct intra-canceral injection, injection into the cancer vasculature or local or regional administration relative to the cancer site.

The active compounds may also be administered parenterally or intraperitoneally. Solutions of the active compounds as free base or pharmacologically acceptable salts can be prepared in water suitably mixed with a surfactant, such as hydroxypropylcellulose. Dispersions can also be prepared in glycerol, liquid polyethylene glycols, and mixtures thereof and in oils. Under ordinary conditions of storage and use, these preparations contain a preservative to prevent the growth of microorganisms.

The pharmaceutical forms suitable for injectable use include sterile aqueous solutions or dispersions and sterile powders for the extemporaneous preparation of sterile injectable solutions or dispersions. In all cases the form must be sterile and must be fluid to the extent that easy syringability exists. It must be stable under the conditions of manufacture and storage and must be preserved against the contaminating action of microorganisms, such as bacteria and fungi. The carrier can be a solvent or dispersion medium containing, for example, water, ethanol, polyol (for example, glycerol, propylene glycol, and liquid polyethylene glycol, and the like), suitable mixtures thereof, and vegetable oils. The proper fluidity can be maintained, for example, by the use of a coating, such as lecithin, by the maintenance of the required particle size in the case of dispersion and by the use of surfactants. The prevention of the action of microorganisms can be brought about by various antibacterial an antifimgal agents, for example, parabens, chlorobutanol, phenol, sorbic acid, thimerosal, and the like. In many cases, it will be preferable to include isotonic agents, for example, sugars or sodium chloride. Prolonged absorption of the injectable compositions can be brought about by the use in the compositions of agents delaying absorption, for example, aluminum monostearate and gelatin.

Sterile injectable solutions are prepared by incorporating the active compounds in the required amount in the appropriate solvent with various of the other ingredients enumerated above, as required, followed by filtered sterilization. Generally, dispersions are prepared by incorporating the various sterilized active ingredients into a sterile vehicle which contains the basic dispersion medium and the required other ingredients from those enumerated above. In the case of sterile powders for the preparation of sterile injectable solutions, the preferred methods of preparation are vacuum-drying and freeze-drying techniques which yield a powder of the active ingredient plus any additional desired ingredient from a previously sterile-filtered solution thereof.

Pharmaceutical compositions for oral administration are formulated using pharmaceutically acceptable carriers well known in the art in dosages suitable for oral administration. Such carriers enable the pharmaceutical compositions to be formulated as tablets, pills, dragees, capsules, liquids, gels, syrups, slurries, suspensions and the like, for ingestion by the patient.

Pharmaceutical preparations for oral use are obtained through a combination of active compounds with solid excipient, optionally grinding a resulting mixture, and processing the mixture of granules, after adding suitable auxiliaries, if desired, to obtain tablets or dragee cores. Suitable excipients are carbohydrate or protein fillers such as sugars, including lactose, sucrose, mannitol, or sorbitol; starch from corn, wheat, rice, potato, or other plants; cellulose such as methyl cellulose, hydroxypropylmethyl-cellulose, or sodium carboxymethyl cellulose; and gums including arabic and tragacanth; and proteins such as gelatin and collagen. If desired, disintegrating or solubilizing agents may be added, such as the cross-linked polyvinyl pyrrolidone, agar, alginic acid, or a salt thereof, such as sodium alginate.

Dragee cores are provided with suitable coatings such as concentrated sugar solutions, which may also contain gum arabic, talc, polyvinylpyrrolidone, carbopol gel, polyethylene glycol, and/or titanium dioxide, lacquer solutions, and suitable organic solvents or solvent mixtures. Dyestuffs or pigments may be added to the tablets or dragee coatings for product identification or to characterize the quantity of active compound, i.e., dosage.

Pharmaceutical preparations which are used orally include push-fit capsules made of gelatin, as well as soft, sealed capsules made of gelatin and a coating such as glycerol or sorbitol. Push-fit capsules can contain active ingredients mixed with a filler or binders such as lactose or starches, lubricants such as talc or magnesium stearate, and, optionally, stabilizers. In soft capsules, the active compounds may be dissolved or suspended in suitable liquids, such as fatty oils, liquid paraffin, or liquid polyethylene glycol with or without stabilizers.

For nasal administration, penetrants appropriate to the particular barrier to be permeated are used in the formulation. Such penetrants are generally known in the art.

The pharmaceutical compositions of the present invention may be manufactured in a manner known in the art, e.g. by means of conventional mixing, dissolving, granulating, dragee-making, levitating, emulsifying, encapsulating, entrapping or lyophilizing processes.

After pharmaceutical compositions comprising a therapeutic agent of the invention formulated in a acceptable carrier have been prepared, they are placed in an appropriate container and labeled for treatment of an indicated condition with information including amount, frequency and method of administration.

The exact dosage is chosen by the individual physician in view of the patient to be treated. Dosage and administration are adjusted to provide sufficient levels of the active moiety or to maintain the desired effect. Additional factors which may be taken into account include the severity of the disease state (e.g., location of the disease, age, weight, and gender of the patient, diet, time and frequency of administration, drug combination(s), reaction sensitivities, and tolerance/response to therapy). Long acting pharmaceutical compositions might be administered every 3 to 4 days, every week, or once every two weeks depending on half-life and clearance rate of the particular formulation. General guidance as to particular dosages and methods of delivery for other applications is provided in the literature (see U.S. Pat. Nos. 4,657,760; 5,206,344; and 5,225,212, herein incorporated by reference). Those skilled in the art will typically employ different formulations for oligonucleotides and gene therapy vectors than for proteins or their inhibitors. Similarly, delivery of polynucleotides or polypeptides will be specific to particular cells, conditions, locations, and the like.

Animal Models

There are a number of animal models for cancer that can be used to test and adjust the compositions and methods of this invention. Certain models involve injecting in-bred animals with established syngeneic tumor lines. The tumors can be co-injected with a potentially therapeutic composition, allowed to establish before therapy is commenced, or administered as a challenge at some time following vaccination of a naive animal. Illustrations are provided in the Example section. Also useful are chimeric animal models, described in U.S. Pat. Nos. 5,663,481, 5,602,305 and 5,476,993; EP application 379,554; and International application WO 91/01760.

### EXAMPLES

### Example 1

### Yeast Two-Hybrid Screen for Pin2 Binding Polypeptides and the Cloning of PinX1

To screen for Pin2 binding polypeptides, a yeast two-hybrid screen was carried out, as described (Lu et al., 1996). Briefly, the cDNA encoding the Pin2 isoform of Pin2/TRF1 was fused to the GAL4 DNA-binding domain in the pAS2 vector and transformed into the Y190 yeast strain to establish stable transformants. The stable strains were transformed again with a HeLa cDNA library fused to the GAL4 activation domain in the pGAD-GH vector, followed by His and LacZ screening. Of the 10⁸ yeast colonies screened, about 500 clones were positive and 273 of them confirmed to be strongly positive on the secondary screen. The cDNA inserts were recovered and sequenced. An additional 5′ or 3′ sequences were obtained by screening a HeLa cell cDNA library, as described (Lu et al., 1996).

Out of 10⁸ clones screened, we obtained 274 strongly positive clones after two runs of the screen. From sequencing, we identified a total of six known genes and four unknown genes. The known genes included the Pin2 isoform (two clones), nm23-H1 (eleven) and Tin2 (five). Since Pin2/TRF1 has been shown to form dimers (Bianchi et al., 1997; Shen et al., 1997), and to interact with Tin2 and nm23-H1 (Kim et al., 1999; Nosaka et al., 1998), these results validate our interaction screen. PinX1 is one of the four unknown Pin2/TRF1-interacting protein Xs (PinX1-4).

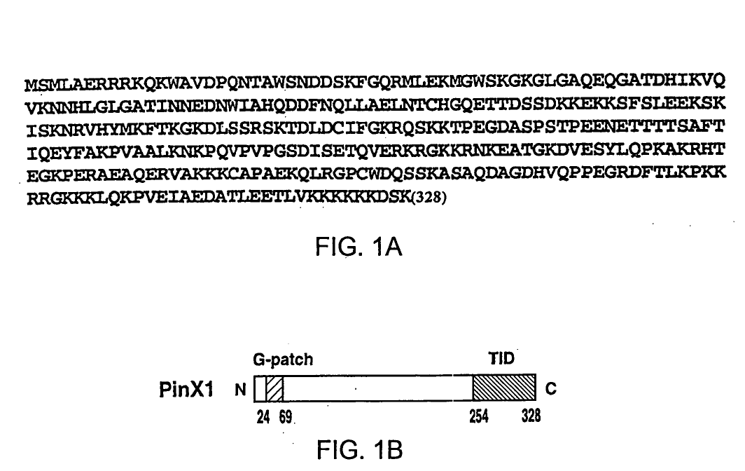

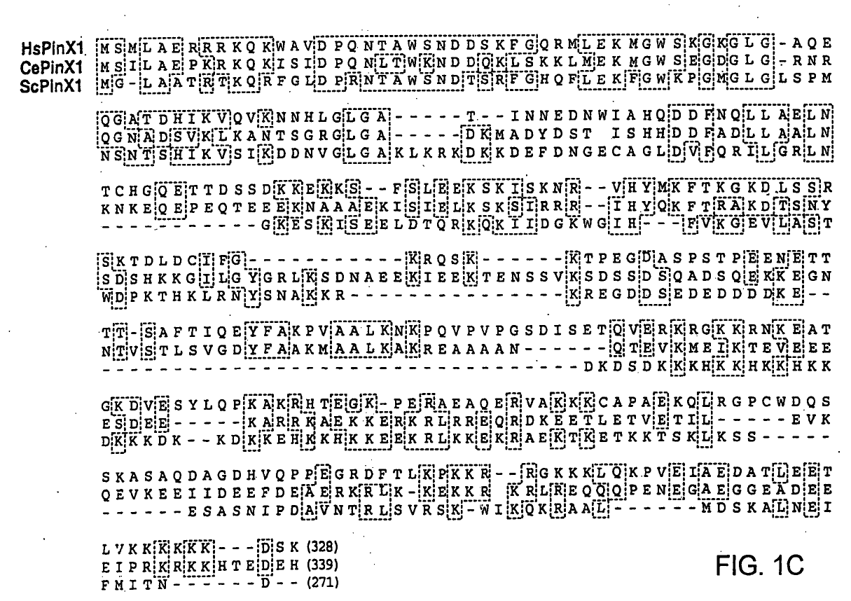

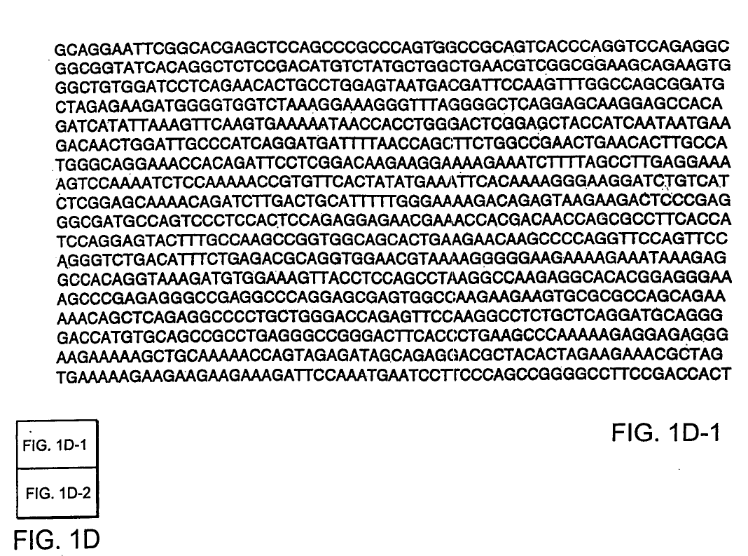

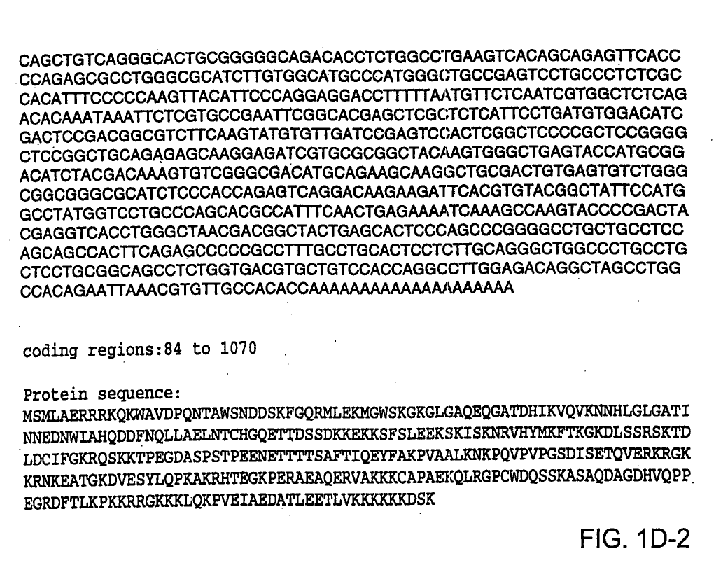

Three positive clones contained PinX1 inserts that overlapped in sequence and the longest 1878 bp cDNA clone contained a 984 bp open reading frame (ORF) encoding a 328-amino acid protein (FIG. 1A). A database search revealed no known domain structure in PinX1 with the exception of a Gly-rich patch located between amino acid 24 to 69 (FIG. 1B), which in other proteins has been hypothesized to bind RNA, but its binding activity or function has not been shown (Aravind and Koonin, 1999). A GenBank database search revealed PinX1 ORFs present in the genome of other eukaryotic cells including the budding yeast and C. elegans, which encode similar numbers of amino acids with an overall ˜30% identity (˜50% similarity) to the human protein (FIG. 1C). These results indicate that PinX1 proteins are conserved.

The human PinX1 gene localizes to chromosome 8p23 close to the microsatellite marker D8S277, based on the human genome sequence (http://www.ncbi.nlm.nih.gov/genemap99/map.cgi?CHR=8) (Deloukas et al., 1998). Interestingly, loss of heterozygosity (LOH) at 8p23 has been shown to occur at a high frequency in a number of human cancers including liver, breast, colorectal, prostate, lung, head and neck, pancreatic and urinary bladder carcinomas (Baffa et al., 2000; Bockmuhl et al., 2001; Emi et al., 1992; Ishwad et al., 1999; Matsuyama et al., 1994; Nielsen and Briand, 1989; Perinchery et al., 1999; Pineau et al., 1999; Shao et al., 2000; Sun et al., 1999), suggesting that PinX1 may be a potential tumor suppressor.

### Example 2

### Identification of Full Length PinX1 cDNA

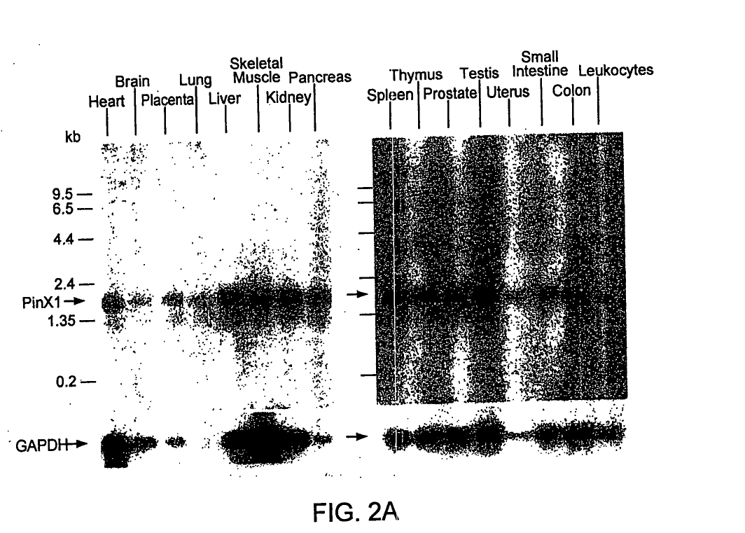

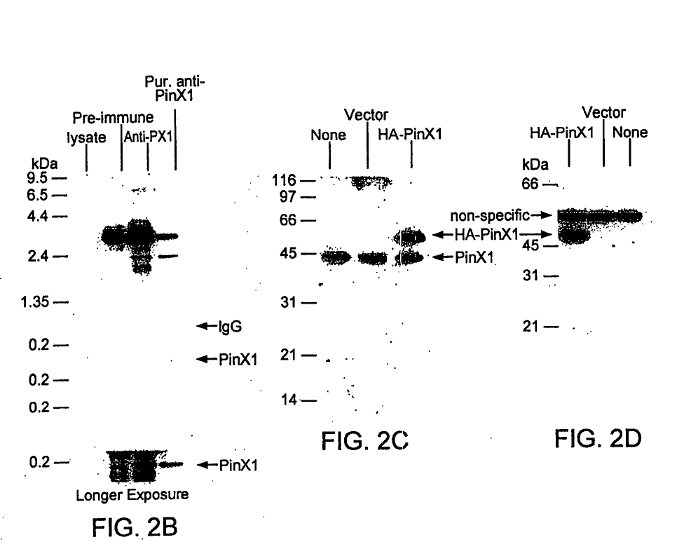

To verify that the PinX1 cDNA encodes a full length protein that is expressed in human cells, we first examined expression of the PinX1 gene in human tissues by Northern analysis of various normal human tissues. A single 1.9 kb PinX1 mRNA transcript was detected in all 16 human tissues examined at various levels, with relatively high expression being observed in liver, kidney, spleen and testis, and low expression in brain and peripheral blood leukocytes (FIG. 2A). These results support that the isolated PinX1 cDNA is the full length sequence and indicate the ubiquitous expression of PinX1 in human tissues.

### Example 3

### Production of Anti-PinX1 Antibodies and Immunostaining

We raised anti-PinX1 polyclonal antibodies against recombinant glutathione-S-transferase (GST)-PinX1 fusion protein to detect endogenous PinX1 protein in cells. To raise antibodies against PinX1, recombinant GST-PinX1 was used to immunize two rabbits, as described (Lu et al., 1996). To purify the antibodies, GST-PinX1 was covalently coupled to glutathione beads and used as an affinity column for purifying the anti-PinX1 antibodies, as described (Lu et al., 1999). Immunostaining using affinity-purified PinX1 antibodies or anti-HA antibody (12CA5) was performed, as described (Lu et al., 1996; Lu and Hunter, 1995).

GST pulldown, immunoprecipitation and immunoblotting analysis were performed as described (Lu et al., 1999; Shen et al., 1998). Briefly, relevant proteins were expressed in HeLa or HT1080 cells by transient transfection, or translated in vitro using the TNT coupled transcription/translation kit (Promega) in the presence of [³⁵S]-Met, followed by lysis or dilution in a buffer containing 50 mM HEPES, pH 7.5, 150 mM NaCl, 100 mM NaF, 1 mM sodium orthovanadate, 10% glycerol, 1% Triton X100, 10 μg/ml aprotinin, 10 μg/ml leupeptin, 50 μg/ml phenylmethylsulfonyl fluoride and 1 mM DTT. The cellular supernatants were incubated with 1 μM GST or GST fusion proteins for 1 hr at 4° C. and 15 μl of glutathione agarose beads were then added, followed by further incubation for 2 h at 4° C. The precipitated proteins were washed 4 times in the same buffer and subjected to immunoblotting analysis. Other antibodies used for immunoprecipitation and immunoblotting analysis include anti-Pin2/TRF1 (Shen et al., 1997) and anti-hTERT antibodies (Novus). Membranes containing RNAs isolated from different human tissues (Clontech) were hybridized to the C-terminal coding sequence of PinX1 and then stripped, followed by reprobing with the house-keeping gene GAPDH for loading control.

When human HeLa cell lysates were subjected to immunuprecipiation, followed by immunoblotting analysis with anti-PinX1 antibodies, only the anti-PinX1 sera, but not the pre-immune control, specifically immunoprecipitated a single 45 kDa protein (FIG. 2B). The same protein was also immunoprecipitated by anti-PinX1 antibodies that were affinity purified using the GST-PinX1 column (FIG. 2B). In addition, when the anti-PinX1-specific antibodies in the sera were first depleted using the GST-PinX1 column, the depleted sera failed to immunoprecipitate the 45 kDa protein from HeLa cells. These results indicate that anti-PinX1 antibodies specifically recognize a 45 kDa protein.

### Example 4

### Expression and Purification of Recombinant Proteins

To generate an N-terminally GST- or His-PinX1 fusion proteins, cDNAs encoding full length PinX1 and its mutants were subdloned into a pGEX or pET28a vector, respectively, and the resulting fusion proteins expressed and purified by glutathione or Ni²⁺-NTA agarose column, as described (Lu et al., 1999; Zhou et al., 2000). GST-Pin2 proteins were produced and purified, as described (Shen et al., 1997).

Since the 45 kDa molecular weight is slightly bigger than that predicted from the deduced sequence, which is 36,958 Da, we needed to confirm that this 45 kDa protein is PinX1. We therefore expressed PinX1 in HeLa cells with an N-terminal HA epitope tag, and subjected it to immunoblotting analysis with anti-PinX1 antibodies or anti-HA antibody. Both anti-PinX1 and anti-HA antibodies recognized a ˜50 kDa protein only in PinX1-transfected cells, but not in non-transfected or vector-transfected cells (FIG. 2C, D). In addition, the same 50 kDa HA-PinX1 protein was produced when synthesized by in vitro transcription and translation (data not shown). Since the molecular weight of the HA tag plus linker sequences is expected to be about 5 kDa, these results indicate that PinX1 encodes a 45 kDa protein that is expressed in human cells.

### Example 5

### Establishment of Stably PinX1 Transfected HT1080 Cell Lines

To generate stable cell lines, the HA-PinX1 cDNA was subcloned in an expression vector in a sense or antisense orientation and then were transfected into fibrosarcoma cell line HT1080, along with the vector as a control, as described (Lu et al., 1996). To generate stable cell lines expressing PinX1 mutants, PinX1-N and PinX1-C expression constructs were transfected into HT1080 cells. After selection with antibiotics and limiting dilution, multiple independent single clones were isolated and checked for protein expression by immunoblotting analysis with anti-HA or anti-PinX1 antibodies. Although several clones expressing PinX1-N were obtained initially, they all lost expression during expansion. However, multiple independent stable clones with other constructs expressed the expected proteins and exhibited similar properties.

### Example 6

### Growth Curves and Phenotypic Analysis of Stable Cell Lines

To monitor growth property and morphology of these stable cell lines, we maintained the stable cell lines continuously in culture, splitting on every fourth day and seeding at the concentration of 6×105 cells per 10 cm culture dish. Cell growth curves were determined by counting the cell number at each subculture and cell morphology was observed under a microscope. In addition, cell proliferation was assayed by incubation with 10 μM BrdU for 30 min and incorporation of BrdU into cells was determined by staining cells with FITC-conjugated anti-BrdU monoclonal antibody according to the manufacture's protocol (Pharmingen), followed by flow cytometry. To detect phenotype of the rounded and loosely attached cells in PinX1-C-expressing cells, they were harvested by aspiration and fixed in 70% ethanol. After incubation with propidium iodide (10 μg/ml) and 250 μg/ml of ribonuclease A, the DNA content was determined by flow cytometry analysis (Becton-Dickinson), as described (Kishi et al., 2000; Lu and Hunter, 1995). To stain for senescence-associated β-galactosidase (SA-β-Gal), cells grown in dishes or on coverslips were washed and then fixed with 2% formaldehyde/0.2% glutaraldehyde in PBS for 5 min at room temperature. SA-β-gal (pH 6.0) was detected, as reported previously (Dimri et al., 1995). Cells were rinsed in PBS, followed by determining the staining and cell morphology under a microscope.

### Example 6

### PinX1 Interacts with Pin2/TRF1 in vivo and in vitro

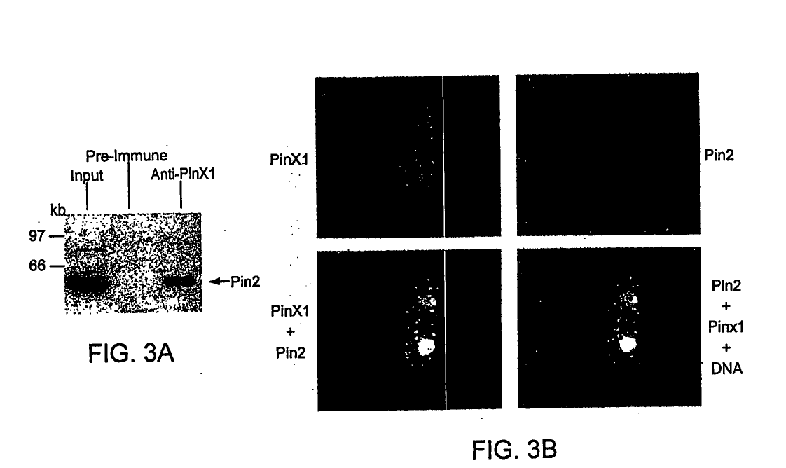

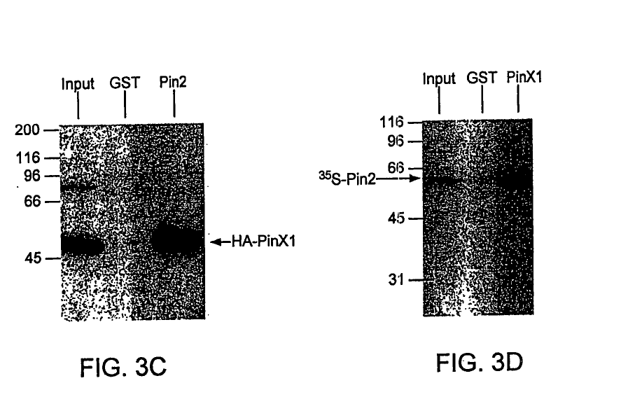

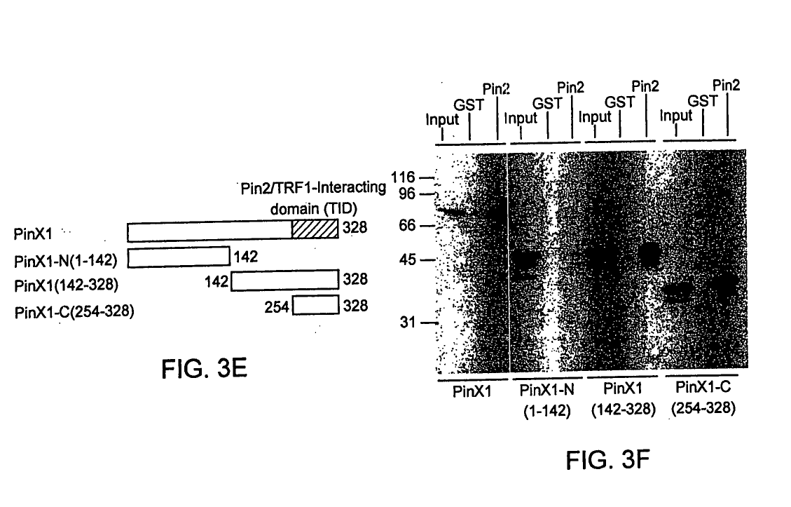

Since PinX1 was originally isolated as a Pin2/TRF1-interacting protein in the yeast two-hybrid screen, it suggested that PinX1 might interact with Pin2/TRF1. To confirm this apparent interaction, we performed co-immunoprecipitation and co-localization experiments. HeLa cells were co-transfected with PinX1 and Pin2 expression constructs, and then subjected to immunoprecipitation with anti-PinX1 or preimmune sera, followed by immunoblotting with anti-Pin2/TRF1 antibodies. Pin2 was detected in anti-PinX1 immunoprecipitates, but not in the preimmune control (FIG. 3A), indicating that PinX1 forms stable complexes with Pin2/TRF1 in cells.

To examine whether PinX1 co-localizes with Pin2/TRF1 in cells, we co-transfected HeLa cell with GFP-PinX1 (green) and RFP-2 (red), or HA-PinX1 and GFP-Pin2, and then examined their subcellular localization directly or after immunostaining with anti-HA or affinity-purified anti-PinX1 antibodies. Although anti-PinX1 antibodies failed to detect the endogenous protein likely due to very low level of PinX1 expression in established cell lines, they readily immunostained the transfected PinX1 protein (data not shown). Both HA-PinX1 and GFP-PinX1 colocalized with Pin2/TRF1 in the nucleus, especially at the nuclear speckles and in nucleoli (FIG. 3B, data not shown). Since multiple studies have established that these Pin2/TRF1 speckles are telomeres (Chong et al., 1995; Kim et al., 1999; Shen et al., 1997), these results suggest that PinX1 co-localizes with Pin2/TRF1 at telomeres. These results indicate that PinX1 and Pin2/TRF1 not only co-immunoprecipitate, but also co-localize in cells.

Having demonstrated in vivo association, we performed in vitro binding assays using GST pulldown experiments, as described previously (Lu et al., 1999; Shen et al., 1998). HeLa cells were transfected with HA-PinX1 and then incubated with glutathione beads containing GST or GST-Pin2, followed by subjecting bound proteins to immunoblotting analysis with anti-HA antibody. Only the GST-Pin2, but not GST, specifically precipitated HA-PinX1 (FIG. 3C). Conversely, when in vitro translated [35S]-Pin2 was incubated with glutathione beads containing GST or GST-PinX1, only PinX1, but not GST precipitated [35S]-Pin2 (FIG. 3B). These results indicate that PinX1 interacts with Pin2 in vitro.

### Example 7

### Identification of Pin2 Binding Domain on PinX1

To map the region in PinX1 that interacts with Pin2, we expressed different PinX1 fragments in HeLa cells as GFP fusion proteins (FIG. 3E), and then subjected them to GST pulldown experiments with GST-Pin2. Although there was no binding between GST and any PinX1 fragments, GST-Pin2 bound PinX1, its C-terminal 142-328 and 254-328 amino acid fragments (FIG. 3F). In contrast, Pin2 did not bind the N-terminal 142 amino acid fragment of PinX1 (FIG. 3F). These results indicate that the C-terminal 74 amino acids of PinX1 are required for binding Pin2/TRF 1. For clarity, we referred the N-terminal 142 and C-terminal 74 amino acid fragments of PinX1 as PinX1-N and PinX1-C, respectively (FIG. 3E).

### Example 8

### Overexpression of PinX1 Induces a Fraction of Transformed HT1080 Cells to Enter Senescence-Like State and PinX1-C Forces Most Cells into Crisis

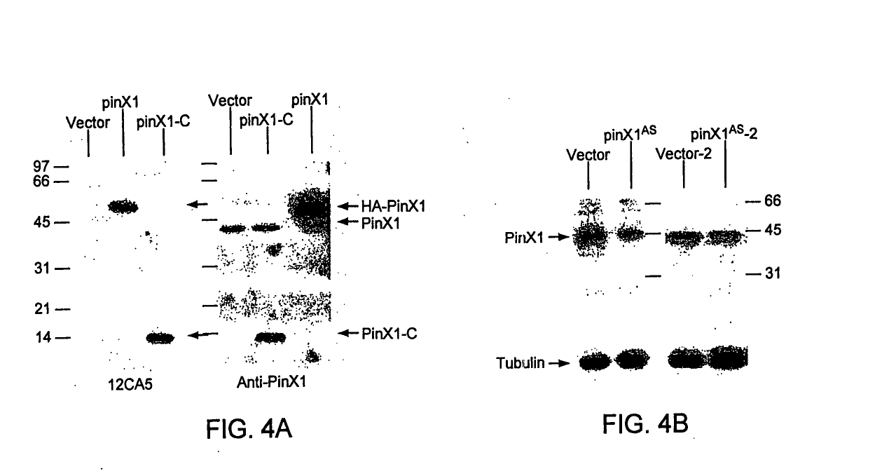

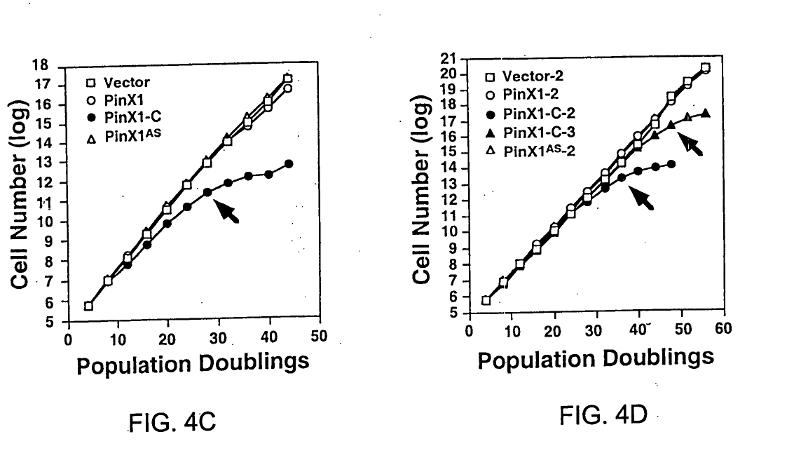

Pin2/TRF1 and its binding proteins, tankyrase and Tin2, have been shown to regulate telomere length in telomerase-positive fibrosarcoma cell line HT1080 (Kim et al., 1999; Smith et al., 1998; van Steensel and de Lange, 1997). To examine whether PinX1 affects telomere maintenance, we tried to establish HT1080 cell lines stably expressing PinX1, PinX1-N or -C. To deplete endogenous PinX1 protein, we also expressed the full length PinX1 in an antisense orientation (PinX1AS). After multiple attempts, we could not obtain cell lines stably expressing PinX1-N. However, we were able to establish multiple independent cell lines that stably expressed PinX1, PinX1-C or PinX1 AS, along with cell lines that were stably transfected with the control vector (FIG. 4A, B). Importantly, expression of PinX1AS in HT1080 cells resulted in significant decreases in the level of endogenous PinX1 protein in multiple clones, as compared with those in vector control clones (FIG. 4B). These results demonstrate that we are able to manipulate PinX1 protein levels in stable HT1080 clones.

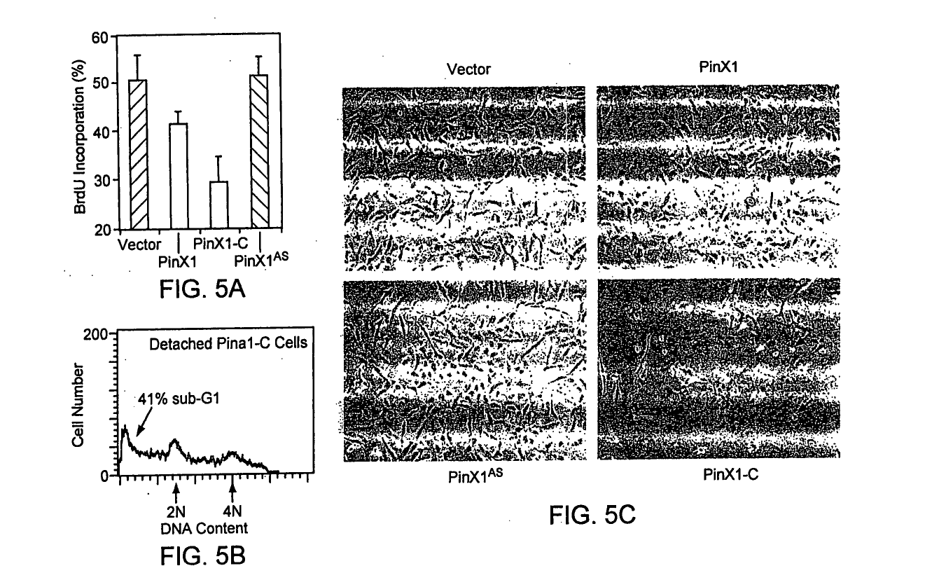

To examine the effects of overexpression and depletion of PinX1 on growth property and morphology, we maintained these stable cell lines continuously in culture, splitting on every fourth day and seeding at the concentration of 6×10⁵ cells per 10 cm culture dish. Cells expressing PinX1AS and control vector grew normally and there was no detectable difference in the growth rate or cell morphology over the period of about 60 population doublings (PD) (FIG. 4C, D, FIG. 5). Initially, all PinX1 or PinX1-C stable cell lines grew at the same rates as those of vector control cells (FIG. 4C, D), indicating that these proteins do not have non-specific toxic effects on cell growth. After about 20-30 PD, the growth of PinX1-expressing cells slightly slowed down, as indicated by a slight decrease in BrdU incorporation, as compared with vector or PinX1AS cells (FIG. 5A). Notably, on third day after subculture when vector-transfected cells usually were at about 90% confluency, the PinX1-expressing cells were only at about 75% confluency (data not shown). Interestingly, a fraction of cells exhibited morphological characteristics that were more typical of senescent cells. These cells exhibited increased size and a flattened morphology (FIG. 5C). Furthermore, they also stained positively for the senescence-associated β-galactosidase (SA-β-Gal) (FIG. 5C), a biomarker for identifying senescent human cells in culture and in aging skin in vivo (Dimri et al., 1995). Such huge and SA-β-Gal-positive senescence-like cells were readily detected in multiple independent PinX1-expressing cell lines after 20-30 PD, but were rarely found in the vector- or PinX1AS-expressing cell lines (FIG. 5C, data not shown). However, most other PinX1-expressing cells were able to continue to divide and reach confluency on the fourth day, when they were harvested, counted and propagated further using the same number of cells (6×10⁵ per 10 cm flask. Therefore, there were no obvious effects on overall growth between PinX1-expressing cells and vector control cells (FIG. 4C, D). These results indicate that overexpression of PinX1 causes a fraction of transformed cells to enter a senescence-like state, which likely accounts for the slight decrease in cell proliferation.

A most striking phenotype was observed in PinX1-C-expressing cells. All three PinX1-C-expressing independent cell lines tested underwent crisis characterized by an overall reduction in growth rate, although the exact time point at when the crisis occurred varied slightly in different cell lines, between 24 to 40 PD (FIG. 4C, D). Incorporation of BrdU into PinX1-C-expressing cells was significantly reduced, as compared with that of control vector cells (FIG. 5A). In some instances, reduced growth appeared to be due to an increased rate of cell death, with foci of dying cells being observed among patches of proliferative cells. Cells in this death phase were contracted, rounded and loosely attached to culture flask. When these loosely attached cells were harvested and subjected to flow cytometrical analysis, more than 40% of the cells contained a sub-G1 DNA content (FIG. 5B), a characteristic of apoptotic death. However, in most cases, we observed that the cells exhibited increased size and a flattened morphology with elongated cellular processes (FIG. 5C). Furthermore, these cells were stained positive for the SA-β-Gal (FIG. 5C). Since similar crisis phenotypes characterized by cellular senescence and/or apoptosis have been reported in many other cell types including HeLa cells (Feng et al., 1995; Hahn et al., 1999; Herbert et al., 1999; Zhang et al., 1999). These results suggest that overexpression of PinX1-C forces transformed HT1080 cells into crisis.

### Example 9

### Overexpression of PinX1 Partially and PinX1-C Almost Completely Inhibits Telomerase Activity without Significantly Affecting hTERT Protein Levels in vivo

It has been shown that inhibition of telomerase by the expression of antisense hTR forces transformed HeLa cells into crisis and that telomerase activity is sufficient to allow transformed cells to escape from crisis (Feng et al., 1995; Hahn et al., 1999; Halvorsen et al., 1999; Herbert et al., 1999; Zhang et al., 1999). Our findings that expression of PinX1-C forces transformed HT1080 cells into crisis and PinX1 induces senescence-like state in a fraction of cells suggest that PinX1-C and PinX1 might affect telomerase activity. To examine this possibility, we measured telomerase activity in HT1080 cell lines stably expressing PinX1 or PinX-C or the control vector.

For assaying telomerase activity, cells were harvested and lysed in 1X CHAPS lysis buffer (10 mM Tris-HCl, pH 7.5, 1 mM MgC12, 1 mM EGTA, 0.1 mM benzamidine, 5 mM β-mercaptoethanol, 0.5% CHAPS, 10% glyccro, 0.3 U/μl Rnase inhibitor) for 30 min and telomerase-containing fraction was prepared by centrifugation at 12,000×g for 20 min at 4° C. The telomerase activity was assayed using the TRAP-eze (Telomeric Repeat Amplification Protocol) telomerase detection kit (Intergen), which includes a 36-bp internal standard for semiquantitative measurements, as recommended by the manufacturer. To examine the effect on telomerase activity in vitro, different concentrations of PinX1 proteins were incubated with telomerase for 10 min at 4° C. before subjecting to telomerase extension at room temperature for 30 min. After the extension, samples were subjected to a 94° C. hot start, followed by a two-step PCR (94° C. for 30 s, 60° C. for 30 s) for 30 cycles. Telomerase products were electrophoresed on 10% polyacrylamide gels. After electrophoresis, gels were stained with SYBR Green for 30 min according to the manufacturer's instructions. Telomerase activity was semi-quantified by normalizing the band intensities of the characteristic 6-bp telomerase-specific ladder to that of the 36-bp internal standard using NIH image software, as described (Kim et al., 1994; Wright et al., 1995). Experiments were repeated multiple times with different preparations of telomerase and PinX1 proteins, with similar results being obtained.

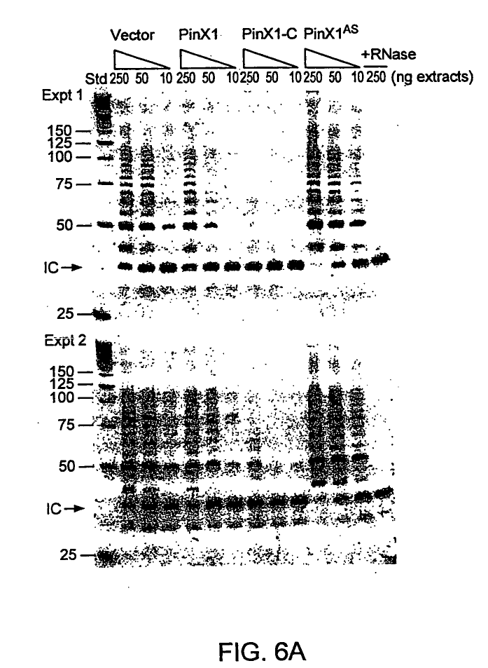

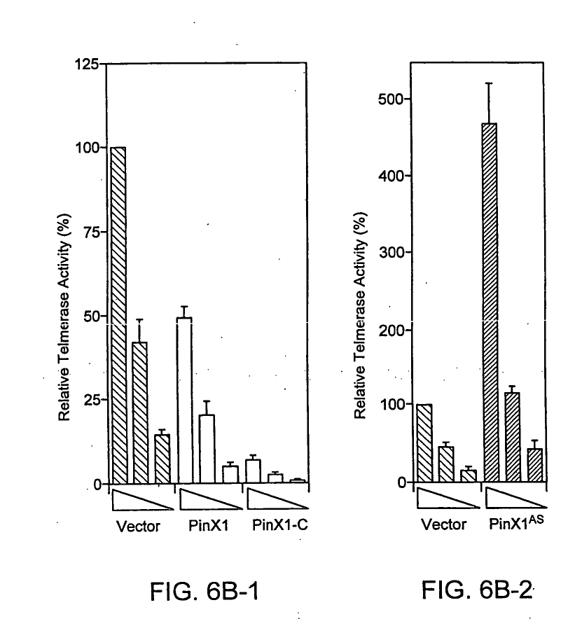

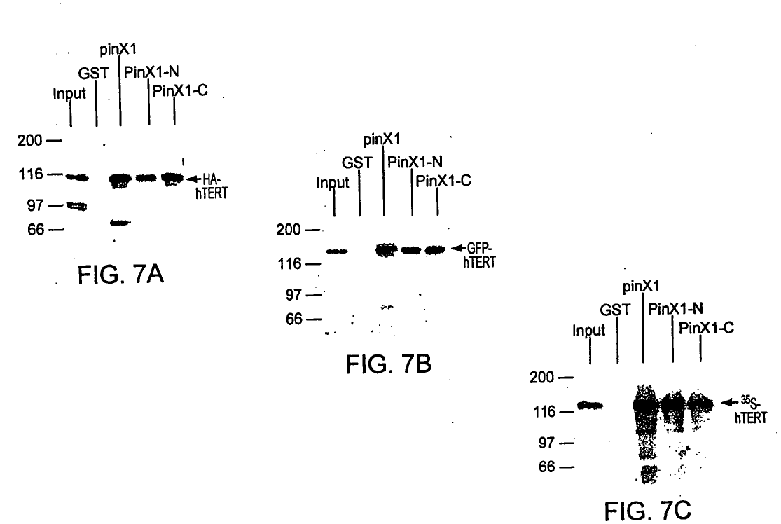

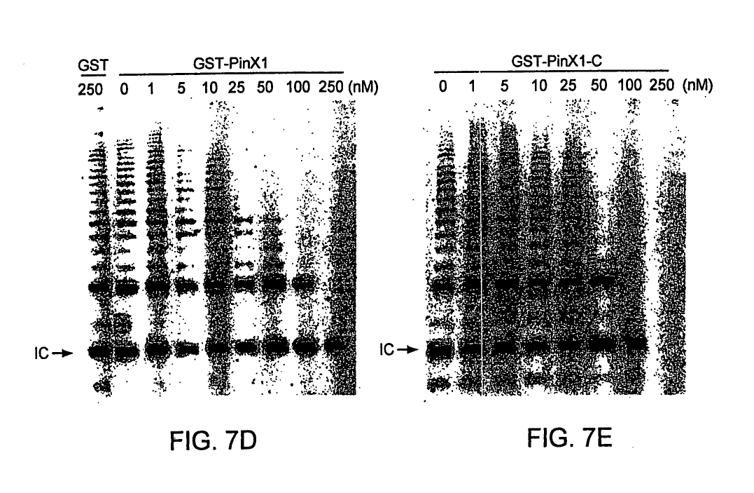

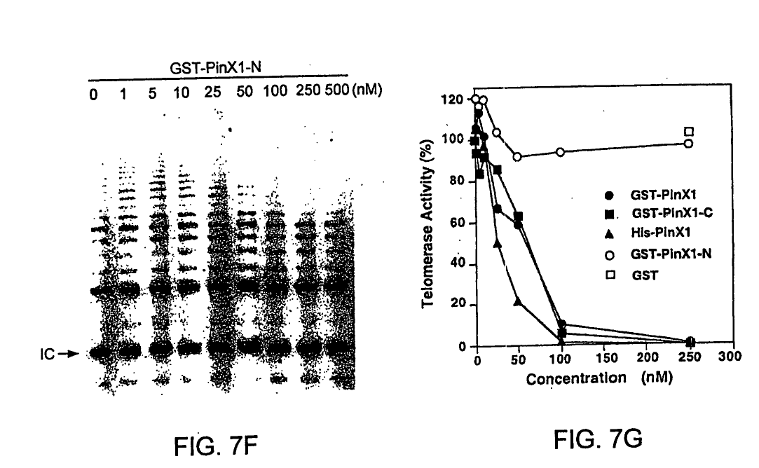

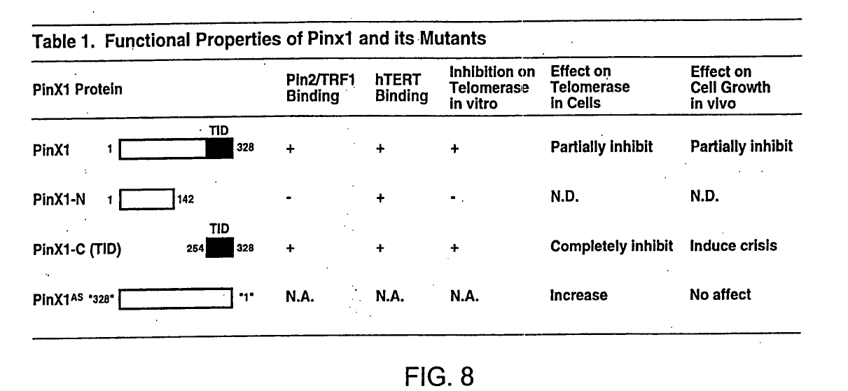

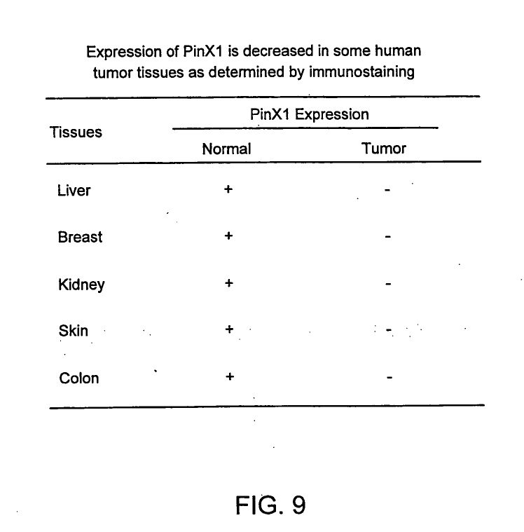

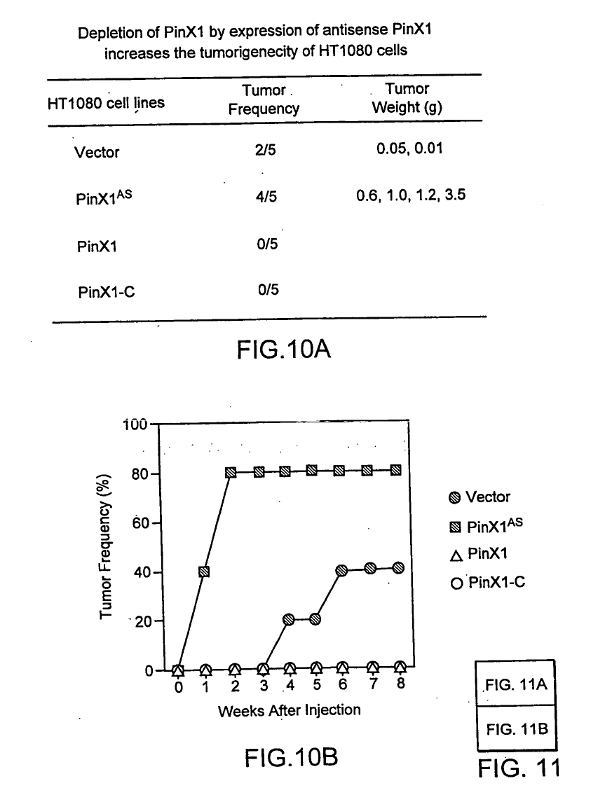

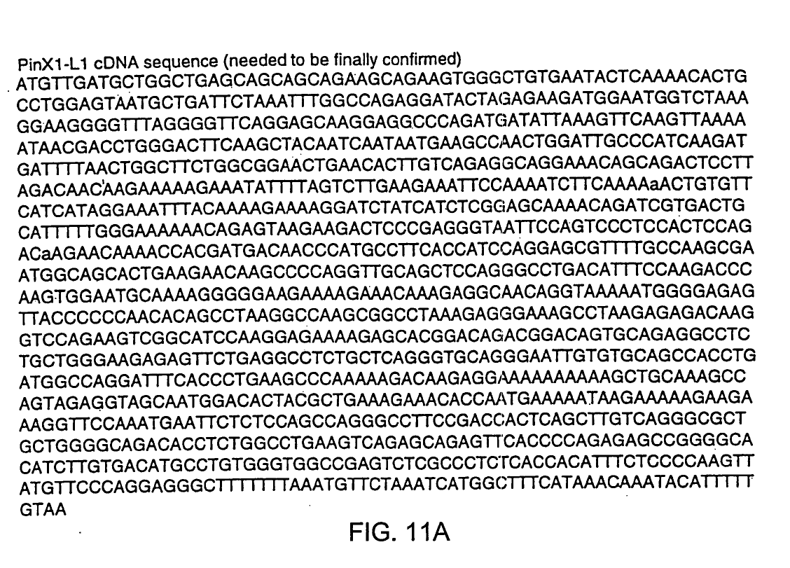

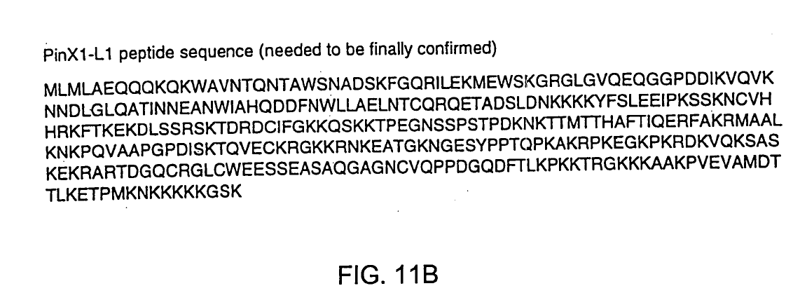

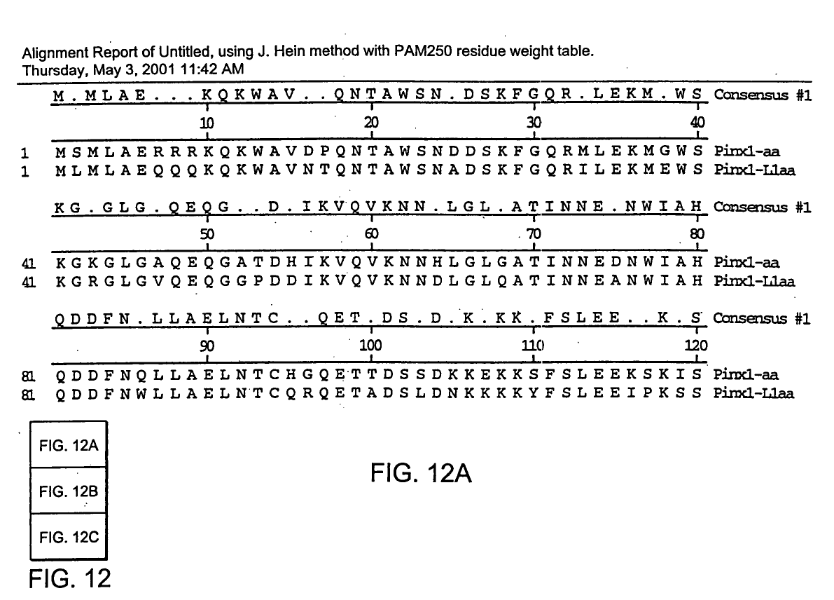

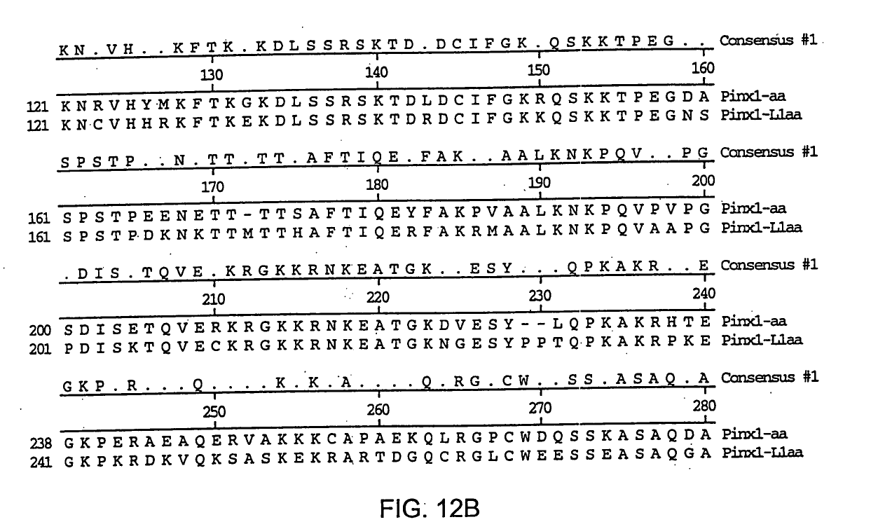

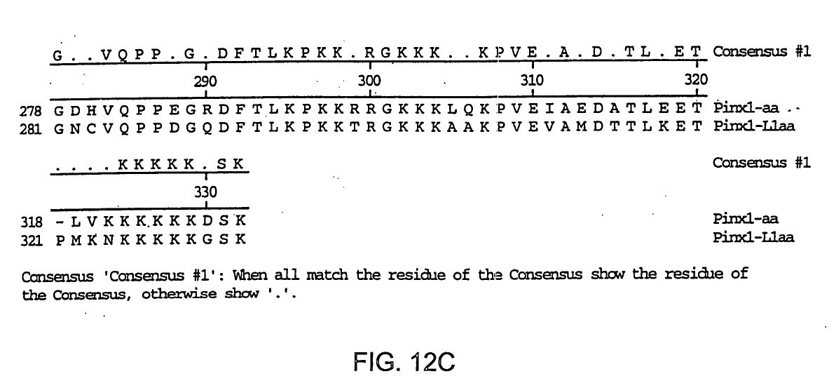

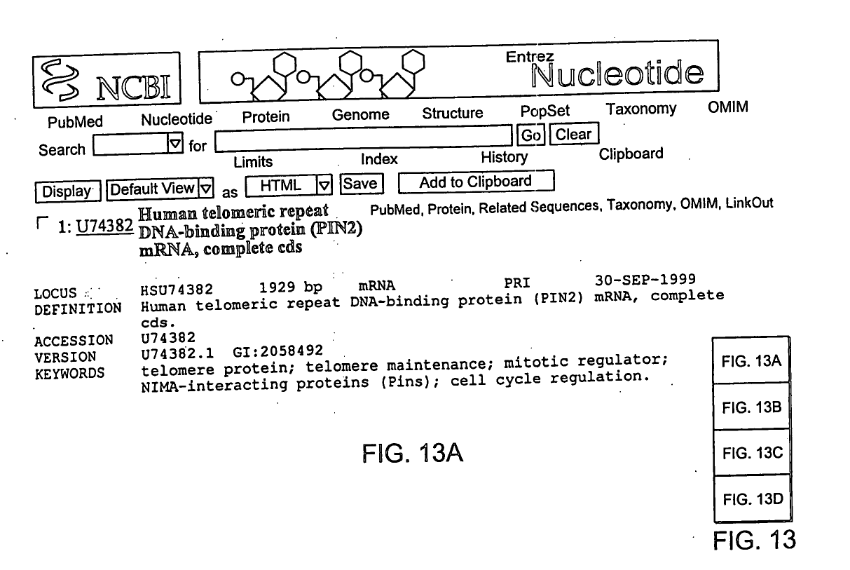

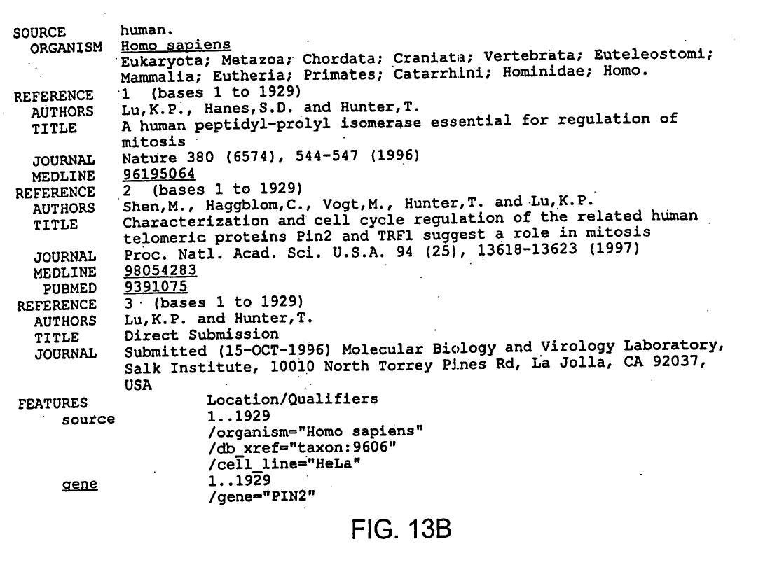

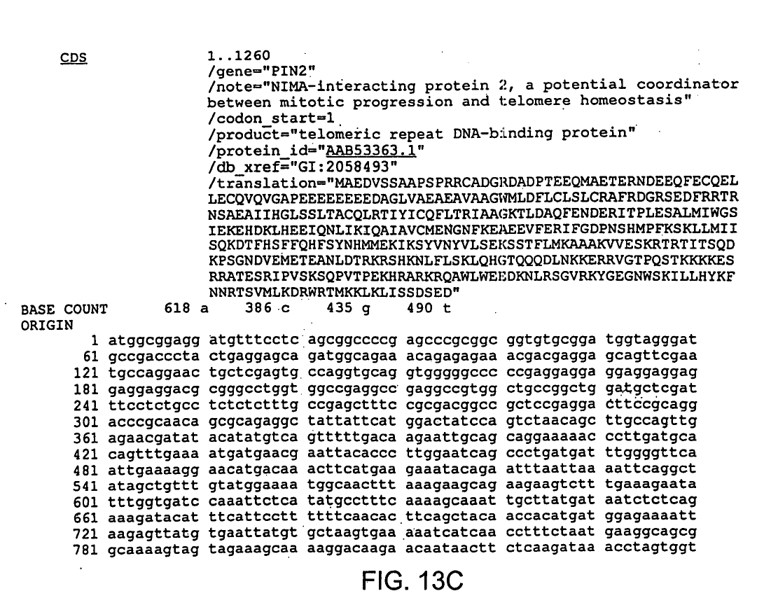

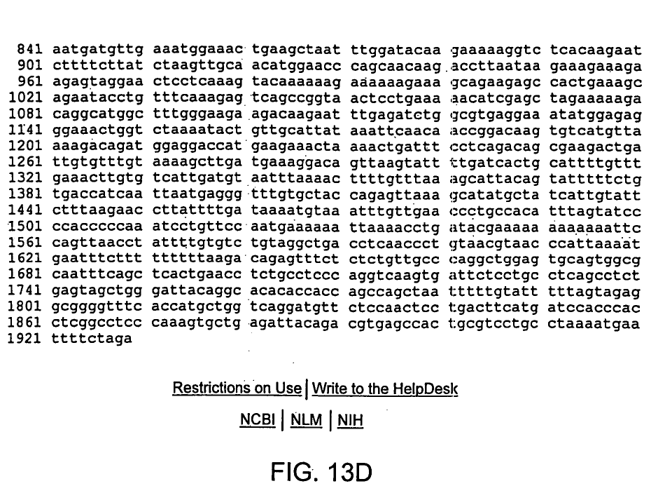

The stable cell lines were analyzed by the standard TRAP assay to measure their telomerase activity. Telomerase activity was readily detected in vector control HT1080 cells (FIG. 6), with the activity similar to that present in parent HT1080 cell. However, no activity was detected if the extracts were heat inactivated or pre-treated with RNase to degrade hTR before telomerase assay prior to the telomerase assay (FIG. 6A). Thus, HT1080 cells contain active telomerase and the control vector has no effect on telomerase activity in cells, as reported previously (Kim et al., 1999; van Steensel and de Lange, 1997). Importantly, as compared with that in vector control cells, telomerase activity in PinX1-stable cells was significantly reduced by about 5 fold, without affecting Tag polymerase used in TRAP assay (FIG. 6), indicating that overexpression of PinX1 significantly inhibits cellular telomerase activity. Most strikingly, telomerase activity was almost not detectable in cells expressing PinX1-C (FIG. 6). Similar inhibitions on telomerase activity were also observed with at least two other independent PinX1- and PinX1-C cell lines examined (data not shown). These results demonstrated that PinX1 partially and PinX1-C almost completely inhibits telomerase activity in cells. This different ability of PinX1 and PinX1-C to inhibit cellular telomerase activity correlates with the ability of PinX1 to induce a fraction of transformed cells to senescence-like state and PinX1-C to force most cells into crisis (FIG. 4, 5).

### Example 10

### Depletion of Endogenous PinX1 Increases Telomerase Activity in vivo

The above results demonstrate that PinX1 inhibits telomerase activity in vivo, suggesting that PinX1 may be an endogenous telomerase inhibitor. If this is the case, depletion of endogenous PinX1 in telomerase-positive cells would result in an increase in cellular telomerase activity. To test this possibility, we assayed hTERT protein level and telomerase activity in HT1080 cell lines that stably expressed PinX1AS and contained less PinX1 protein than that in vector control cells. Telomerase activity in several PinX1AS stable cells was significantly higher than that in the vector-transfected cells (FIG. 6). Interestingly, cellular telomerease activity was increased by about 5 fold in a PinX1AS-expressing cell line where PinX1 was reduced by about 5 fold (FIG. 4B left panel, FIG. 6). These results indicate that depletion of endogenous PinX1 results in an increase in telomerase activity in cells, further confirming that overexpression of PinX1 inhibits activity of cellular telomerase in vivo.

### Example 11

### PinX1 and PinX1-C Potently Binds to Telomerase and Inhibit Telomerase Activity in vitro

The above results show that overexpression of PinX1-C almost completely inhibits and PinX1 significantly reduces telomerase activity, whereas depletion of PinX1 significantly increases telomerase activity in cells. Since the hTERT level was not significantly affected in these stable cell lines, as detected by immunoblotting analysis with anti-hTERT antibodies (data not shown), these results suggest that PinX1 and PinX-C function as telomerase inhibitors in vivo. To further determine the ability of PinX1 and PinX1-C to directly inhibit telomerase, we examined whether they bind hTERT and inhibit telomerase activity in vitro.

To examine the interaction between PinX1 and hTERT, we used GST pulldown experiments as described above. When human TERT was produced either in cells as a HA epitope tagged protein or a GFP fusion protein by transient transfection or synthesized by in vitro transcription and translation, GST-PinX1, but not GST, precipitated hTERT produced by all three procedures (FIG. 7A-C). It appeared that both N- and C-terminal domains of PinX1 precipitated hTERT (FIG. 7A-C). These results indicate that PinX1 can bind hTERT at least in vitro.

To examine the effects of PinX1 proteins on telomerase activity in vitro, telomera-secontaining fraction from normal HT1080 cells was incubated with GST or GST-PinX1 fusion proteins for 10 min on ice, followed by the TRAP assay. Both GST-PinX1 and GST-PinX1-C potently and specifically inhibited telomerase activity in a concentration-dependent manner, with an IC50 of about 50 nM for both proteins (FIG. 7D-G). In contrast, GST had no significant effect on telomerase activity. Furthermore, GST-PinX1-N had no significant effect on telomerase activity even at higher concentrations (FIG. 7F, G), although it bound hTERT (FIG. 7A-C). To insure that the telomerase inhibitory effect of PinX1 is not due to the GST tag, we used His-tag PinX1 fusion proteins in the same assay. As shown in FIG. 7G, His-PinX1 also potently inhibited telomerase with an IC50 of about 25 nM, close to that of GST-PinX1. Although at higher concentrations, these two recombinant proteins could also inhibit Tag polymerase, as indicated by the reduced internal control (IC) signal (FIG. 7D, E), they had no effect at all when expressed in cells (FIG. 6), indicating that the inhibitory effect of PinX1 and PinX1-C on telomerase is rather specific. These results indicate that PinX1 is a potent telomerase inhibitor in vitro. Furthermore, we have identified the domain responsible for the inhibition to be located at its C-terminal 74 amino acid fragment, which is designed the telomerase inhibitory domain (TID).

### Example 12

### Expression of PinX1 is Decreased in Some Human Tumor Tissues as Determined by Immunostaining

Human normal or cancer tissues were immunostated with affinity-purified anti-PinX1 antibodies.

### Example 13

### Depletion of PinX1 by Expression of Antisense PinX Increases the Tumorigenecity of HT1080 Cells

HT1080 cell lines that stably expressed PinX1, PinX1-C, antisense PinX1 (PinX1^{AS}) or control vector were injected to the back of nude mice. The appearance of tumors at the injection sites were monitored weekly, followed by removing the tumors at 8 weeks after injection.

### REFERENCES

Ali, M. et al., Gene Ther., 1:367-384, 1994

Allshire R C, Gosden J R, Cross S H, Cranston G, Rout D, Sugawara N, Szostak J W, Fantes P A, Hastie N D Telomeric repeat from T. thermophila cross hybridizes with human telomeres. Nature. 1988 Apr. 14;332(6165):656-9

Allsopp, R. C., Vaziri, H., Patterson, C., Goldstein, S., Younglai, E. V., Futcher, A. B., Greider, C. W., and Harley, C. B. (1992) Proc. Natl. Acad. Sci. USA, 89, 10114-10118

Angello, J. C., et al., J. Cell. Physiol. 132:125-130 (1987)

Angello, J. C., et al., J. Cell. Physiol. 140:288-294 (1989)

Baffa, R., Santoro, R., Bullrich, F., Mandes, B., Ishii, H., and Croce, C. M. (2000). Definition and refinement of chromosome 8p regions of loss of heterozygosity in gastric cancer. Clin Cancer Res 6, 1372-1377

Bartel et al., 1993, In Cellular Interactions in Development: A Practical Approach, Oxford University Press, Oxford, pp. 153-179; Chien et al., 1991, Proc. Natl. Acad. Sci. USA, 88:9578-9582

Berger and Kimmel, Methods in Enzymology, Volume 152, Guide to Molecular Cloning Techniques (1987), Academic Press, Inc., San Diego, Calif.

Berger et al. ed., “Methods in Enzymology”, Vol. 152, p. 307, Academic Press, New York (1987)

Berger et al. ed., “Methods in Enzymology”, Vol. 152, p. 33 & p. 215, Academic Press, New York (1987)

Bianchi, A., Smith, S., Chong, L., Elias, P., and de Lange, T. (1997). TRF1 is a dimer and bends telomeric DNA. EMBO J. 16, 1785-1794.

Bilaud, T., Koering, C. E., Binet, B. E., Ancelin, K., Pollice, A., Gasser, S. M. & Gilson, E. (1996) Nucleic Acids Res. 24, 1294-1303

Blackburn E H, Greider C W, Henderson E, Lee M S, Shampay J, Shippen-Lentz D. Recognition and elongation of telomeres by telomerase. Genome. 1989;31(2):553-60

Blackburn, 1991 Structure and function of telomeres. Nature. 1991 Apr. 18;350(6319):569-73.

Bockmuhl, U., Ishwad, C. S., Ferrell, R. E., and Gollin, S. M. (2001). Association of 8p23 deletions with poor survival in head and neck cancer. Otolaryngol Head Neck Surg 124, 451-455.

Broccoli, D., Young, J. W., and de Lange, T. (1995). Telomerase activity in normal and malignant hematopoietic cells. Proc Natl Acad Sci USA 92, 9082-9086.

Carter and Samulski, 2000, Int. J. Mol. Med. 6:17-27

Chirgwin J M, Przybyla A E, MacDonald R J, Rutter W J. Isolation of biologically active ribonucleic acid from sources enriched in ribonuclease. Biochemistry. 1979 Nov. 27;1 8(24):5294-9

Chong, L, Van Steensel, B., Broccoli, D., Erdjument, B. H., Hanish, J., Tempst, P. & de Lange, T. (1995) Science 270, 1663-1667

Cole et al., Monoclonal Antibodies and Cancer Theraphy, Alan R Liss Inc., New York N.Y., pp. 77-96 (1985)

Collier and Neidle, 1988 Synthesis, molecular modeling, DNA binding, and antitumor properties of some substituted amidoanthraquinones. J Med Chem. 1988 Apr.;31(4):847-57.

Cote et al., 1983, Proc. Natl. Acad. Sci. 80:2026-2030

Counter C M, Hirte H W, Bacchetti S, Harley C B. Telomerase activity in human ovarian carcinoma. Proc Natl Acad Sci USA. 1994 Apr. 12;91(8):2900-4.

Counter, C. M., Avilion, A. A., LeFeuvre, C. E., Stewart, N. G., Greider, C. W., Harley, C. B., and Bacchetti, S. (1992). Telomere shortening associated with chromosome instability is arrested in immortal cells which express telomerase activity. Embo J 11, 1921-1929

Counter, C. M., Meyerson, M., Eaton, E. N., and Weinberg, R. A. (1997). The catalytic subunit of yeast telomerase. Proc Natl Acad Sci USA 94, 9202-9207.

Cristofalo, V. J. and Kritchevsky, D., Med. Exp. 19:313-320 (1969)

Cristofalo, V. J., et al., Exp. Gerontol. 24:367 (1989)

Dichek D A et al., Circulation 80:1347-53 (1989)

Dimri, G. P., Lee, X., Basile, G., Acosta, M., Scott, G., Roskelley, C., Medrano, E. E., Linskens, M., Rubelj, I., Pereira-Smith, O., and et al. (1995). A biomarker that identifies senescent human cells in culture and in aging skin in vivo. Proc Natl Acad Sci USA 92, 9363-9367.

Emi, M., Fujiwara, Y., Nakajima, T., Tsuchiya, E., Tsuda, H., Hirohashi, S., Maeda, Y., Tsuruta, K., Miyaki, M., and Nakamura, Y. (1992). Frequent loss of heterozygosity for loci on chromosome 8p in hepatocellular carcinoma, colorectal cancer, and lung cancer. Cancer Res 52, 5368-5372.

Feng, J., Funk, W. D., Wang, S. S., Weinrich, S. L., Avilion, A. A., Chiu, C. P., Adams, R. R., Chang, E., Allsopp, R. C., Yu, J., and et, a. l. (1995). The RNA component of human telomerase. Science 269, 1236-1241

Fields et al., 1989, Nature, 340:245-247

Flugelman M Y et al., Circulation 85:1110-17 (1992)

Fritz et al., 1992, Curr. Biol., 2:403-405

Gee et al., in Huber and Carr, Molecular and Immunologic Approaches, Futura Publishing Co, Mt Kisco N.Y. (1994)

Goding, Monoclonal Antibodies: Principles and Practice, pp. 98-118 (N.Y. Academic Press 1983

Greider et al., 1996 in Cellular Aging and Cell Death, Wiley-Liss Inc., New York, N.Y., pp. 123-138

Greider, 1996 Annu. Rev. Biochem., 65:337-365

Grossman, et al. ed., “Methods in Enzymology”, Vol. 12, Parts A & B, Academic Press, New York (1968)

Guarente, L., 1993, Proc. Natl. Acad. Sci. USA, 90:1639-1641)

Gubler et al., “Gene”, Vol. 25, 263-269 (1983)

Hahn, W. C., Counter, C. M., Lundberg, A. S., Beijersbergen, R. L., Brooks, M. W., and Weinberg, R. A. (1999). Creation of human tumour cells with defined genetic elements. Nature 400, 464-468.

Halvorsen, T. L., Leibowitz, G., and Levine, F. (1999). Telomerase activity is sufficient to allow transformed cells to escape from crisis. Mol Cell Biol 19, 1864-1870.

Hanahan, J. Mol. Biol., Vol. 166, p. 557 (1983)

Harley et al., 1990 nature, 345:458-460

Harley et al., 1994 Cold Spring Harbor Symposium on Quantitative Biology, 59:307-315

Harley, C. B., Futcher, A. B., and Greider, C. W. (1990) Nature, 345, 458-460

Harlow and Lane (1988), Antibodies, A Laboratory Manual, Cold Spring Harbor Publications, New York

Harlow, et. al., editors, Antibodies: A Laboratory Manual (1988)

Herbert, B., Pitts, A. E., Baker, S. I., Hamilton, S. E., Wright, W. E., Shay, J. W., and Corey, D. R. (1999). Inhibition of human telomerase in immortal human cells leads to progressive telomere shortening and cell death. Proc Natl Acad Sci USA 96, 14276-14281.

Hiyama et al., 1995a Activation of telomerase in human lymphocytes and hematopoietic progenitor cells. J Immunol. 1995 Oct. 15;155(8):3711-5

Hiyama et al., 1995b Telomerase activity in gastric cancer. Cancer Res. 1995 Aug. 1;55(15):3258-62.

Ishwad, C. S., Shuster, M., Bockmuhl, U., Thakker, N., Shah, P., Toomes, C., Dixon, M., Ferrell, R. E., and Gollin, S. M. (1999). Frequent allelic loss and homozygous deletion in chromosome band 8p23 in oral cancer. Int J Cancer 80, 25-31.

Kennedy et al., (1976) Clin. Chim. Acta 70, 1-31

Kim, N. W., Piatyszek, M. A., Prowse, K. R., Harley, C. B., West, M. D., Ho, P. L., Coviello, G. M., Wright, W. E., Weinrich, S. L., and Shay, J. W. (1994). Specific association of human telomerase activity with immortal cells and cancer [see comments]. Science 266, 2011-2015

Kim, S. H., Kaminker, P., and Campisi, J. (1999). TIN2, a new regulator of telomere length in human cells. Nat Genet 23, 405-412.

Kishi, S., Wulf, G., Nakamura, M., and Lu, K. P. (2000). Telomeric protein Pin2/TRF1 induces mitotic arrest and apoptosis in cells containing short telomeres and is down-regulated in breast tumors. Oncogene 20, 1497-1508.

Konig, P., Giraldo, R., Chapman, L & Rhodes, D. (1996) Cell 85, 125-136

Kosbor et al., 983, Immunol. Today 4:72

Land et al., “Nucleic Acids Res.”, Vol. 9, 2251 (1981)

Leclerc G et al., J. Clin. Invest. 90:936-44 (1992)

Lever et al., 1999, Biochem. Soc. Trans. 27: 841-7

Levy, M. Z., Allsopp, R. C., Futcher, A. B., Grieder, C. W., and Harley, C. B. (1992) J. Mol. Biol., 225, 951-960

Lindsey, J., McGill, N. I., Lindsey, L. A., Green, D. K., and Cooke, H. J. (1991) Mutat. Res., 256, 45-48

Linskens et al., PCT No. WO 96/13610, published May 9, 1996 and incorporated herein by reference

Lu, K. P., and Hunter, T. (1995). Evidence for a NIMA-like mitotic pathway in vertebrate cells. Cell 81, 413-424.

Lu, P. J., Wulf, G., Zhou, X. Z., Davies, P., and Lu, K. P. (1999). The Prolyl isomerase Pin1 restores the function of Alzheimer-associated phosphorylated tau protein. Nature 399, 784-788.

Lu, P. J., Zhou, X. Z., Shen, M., and Lu, K. P. (1999). A function of WW domains as phosphoserine- or phosphothreonine-binding modules. Science 283, 1325-1328.

Maniatis et al., Molecular Cloning: A Laboratory Manual, 2nd Ed., (1989), Cold Spring Harbor, N.Y.

Marshall, E., Science, 269:1050-1055, 1995

Matsuyama, H., Pan, Y., Skoog, L., Tribukait, B., Naito, K., Ekman, P., Lichter, P., and Bergerheim, U.S. (1994). Deletion mapping of chromosome 8p in prostate cancer by fluorescence in situ hybridization. Oncogene 9, 3071-3076.

Morin, G. B. (1989) Cell, 59, 521-529

Mullis and Faloona, 1987, Methods Enzymol., 155: 335

Nielsen. K. V., and Briand, P. (1989). Cytogenetic analysis of in vitro karyotvne evolution in a cell line established from nonmalignant human mammary epithelium. Cancer Genet Cytogenet 39, 103-118.

Nippon Seikagaku Kai (Biochemical Society of Japan) ed., “Shin-Seikagaku Jikken Kouza 2, Kakusan III (Kumikae DNA Gijutsu) (New Lectures on Biochemical Experiments 2, Nucleic Acids III (Recombinant DNA Technique))”, Tokyo Kagaku Dojin, Japan (1992)

Nippon Seikagaku Kai (Biochemical Society of Japan) ed., “Shin-Seikagaku Jikken Kouza 1, Tanpakushitsu VII, Tanpakushitsu Kougaku (New Lectures on Biochemical Experiments 1, Proteins VII, (Protein Engineering))”, Tokyo Kagaku Dojin, Japan (1993)

Nippon Seikagaku Kai (Biochemical Society of Japan) ed., “Zoku-Seikagaku Jikken Kouza 1, Idenshi Kenkyuho II (Lectures on Biochemical Experiments (Second Series; 1), Methods for Gene Study II)”, Tokyo Kagaku Dojin, Japan (1986)

Nosaka, K., Kawahara, M., Masuda, M., Satomi, Y., and Nishino, H. (1998). Association of nucleoside diphosphate kinase nm23-H2 with human telomeres. Biochem Biophys Res Commun 243, 342-348.

Orlandi et al., 1989, Proc. Natl. Acad. Sci. 86: 3833

Perinchery, G., Bukurov, N., Nakajima, K., Chang, J., Hooda, M., Oh, B. R., and Dahiya, R. (1999). Loss of two new loci on chromosome 8 (8p23 and 8q12-13) in human prostate cancer. Int J Oncol 14, 495-500.

Pineau, P., Nagai, H., Prigent, S., Wei, Y., Gyapay, G., Weissenbach, J., Tiollais, P., Buendia, M. A., and Dejean, A. (1999). Identification of three distinct regions of allelic deletions on the short arm of chromosome 8 in hepatocellular carcinoma. Oncogene 18, 3127-3134.

Rhyu et al., 1995 Telomeres, telomerase, and immortality. J Natl Cancer Inst. 1995 Jun. 21;87(12):884-94.

Saiki, et al., Science, Vol. 230, pp. 1350 (1985); R. Saiki, et al., Science, Vol. 239, pp. 487 (1985)

Schurs et al., (1977) Clin. Chim. Acta 81, 1-40

Shao, J. Y., Wang, H. Y., Huang, X. M., Feng, Q. S., Huang, P., Feng, B. J., Huang, L. X., Yu, X. J., Li, J. T., Hu, L. F., Ernberg, I., and Zeng, Y. X. (2000). Genome-wide allelotype analysis of sporadic primary nasopharyngeal carcinoma from southern China. Int J Oncol 17, 1267-1275.

Shay et al., 1992, The two-stage mechanism controlling cellular senescence and immortalization. Exp Gerontol. 1992 July-August; 27(4):383-9

Shen et al., 1997, Characterization and cell cycle regulation of the related human telomeric proteins Pin2 and TRF1 suggest a role in mitosis, Proc. Natl. Acad. Sci. USA, 94: 13618-13623

Shen, M., Haggblom, C., Vogt, M., Hunter, T., and Lu, K. P. (1997). Characterization and cell cycle regulation of related human telomeric proteins Pin2 and TRF1 suggest a role in mitosis. Proc. Natl. Acad. Sci. USA 94, 13618-13623.

Shen, M., Stukenberg, P. T., Kirschner, M. W., and Lu, K. P. (1998). The essential mitotic peptidylprolyl isomerase Pin1 binds and regulates mitosis-specific phosphoproteins. Genes Dev. 12, 706-720.

Smith, S., Giriat, I., Schmitt, A., and de Lange, T. (1998). Tankyrase, a poly(ADP-ribose) polymerase at human telomeres. Science 282, 1484-1487.

Sun, P. C., Schmidt, A. P., Pashia, M. E., Sunwoo, J. B., and Scholnick, S. B. (1999). Homozygous deletions define a region of 8p23.2 containing a putative tumor suppressor gene. Genomics 62, 184-188.

van Steensel, B., and de Lange, T. (1997). Control of telomere length by the human telomeric protein Trf1. Nature 385, 740-743.

Wilson J M et al., Science 244:1344-46 (1989)

Winter and Milstein, 1991, Nature 349:293

Wright, W. E., Shay, J. W., and Piatyszek, M. A. (1995). Modifications of a telomeric repeat amplification protocol (TRAP) result in increased reliability, linearity and sensitivity. Nucleic Acids Res 23, 3794-3795

Wu (ed.), “Methods in Enzymology”, Vol. 68, Academic Press, New York (1980)

Wu et al. (ed.), “Methods in Enzymology”, Vols. 100 & 101, Academic Press, New York (1983)

Wu et al. (ed.), “Methods in Enzymology”, Vols. 153, 154 & 155, Academic Press, New York (1987)

Yegorov, Y. E., Chernov, D. N., Akimov, S. S., Bolsheva, N. L., Kraevsky, A. A., and Zelenin, A. V. (1997) Mol. Biol. (Moscow), 31, 130-136

Zhang, X., Mar, V., Zhou, W., Harrington, L., and Robinson, M. O. (1999). Telomere shortening and apoptosis in telomerase-inhibited human tumor cells. Genes Dev 13, 2388-2399.

Zhou, J., Monson, E. K., Teng, S., Schulz, V. P., and Zakian, V. A. (2000). Piflp helicase, a catalytic inhibitor of telomerase in yeast. Science 289, 771-774.

Zhou, X. Z., Kops, O., Werner, A., Lu, P. J., Shen, M., Stoller, G., Küillertz, G., Stark, M., Fischer, G., and Lu, K. P. (2000). Pin1-dependent prolyl isomerization regulates dephosphorylation of Cdc25C and tau proteins. Mol Cell 6, 873-883

### OTHER EMBODIMENTS

The above examples are included to demonstrate preferred embodiments of the invention. It should be appreciated by those of skill in the art that the techniques disclosed in the examples which follow represent techniques discovered by the inventor to function well in the practice of the invention, and thus can be considered to constitute preferred modes for its practice. However, those of skill in the art should, in light of the present disclosure, appreciate that many changes can be made in the specific embodiments which are disclosed and still obtain a like or similar result without departing from the spirit and scope of the invention.

1. An isolated PinX1 polynucleotide comprising a sequence of SEQ ID No. 1 or SEQ ID No. 2,

2. An isolated PinX1-L1 polynucleotide comprising a sequence of SEQ ID No. 5.

3. A vector comprising the polynucleotide of claim 1 or 2.

4. A host cell comprising the DNA vector of claim 3.

5. The isolated polynucleotide of claim 1 or 2, said polynucleotide molecule being covalently coupled with a detectable label.

6. The isolated polynucleotide of claim 5, wherein said detectable label is one selected from the group consisting of: radiolabel, fluorescent label, chemiluminescent label and calorimetric label.

7. An isolated PinX1 polypeptide comprising SEQ ID No. 3 or SEQ ID No. 4.

8. An isolated PinX1-L1 polypeptide comprising SEQ ID No. 6.

9. A polyclonal antibody specifically immunoreactive with a PinX1 polypeptide comprising SEQ ID No. 3 or SEQ ID No. 4.

10. A monoclonal antibody specifically immunoreactive with a PinX1 polypeptide comprising SEQ ID No. 3 or SEQ ID No. 4.

11. A mouse hybridoma cell line for generating the monoclonal antibody of claim 10.

12. The antibody of claim 9 or 10, said antibody being covalently coupled with a detectable label.

13. The antibody of claim 12, wherein said detectable label is one selected from the group consisting of: radiolabel, fluorescent label, chemiluminescent label and calorimetric label.

14. A method for diagnosis of a cancerous or precancerous condition in a mammal, said method comprising performing a detection step to detect a hybrid formed between a probe and a biological sample from said mammal, said probe comprising a sequence complementary to 15 or more consecutive nucleotide sequence of SEQ ID No. 1 or SEQ ID No. 5, wherein the absence of a detectable hybrid is indicative of said cancerous or precancerous condition.

15. The method of claim 14, further comprising the step of comparing the amount of said hybrid detected in said biological sample with the amount of a control hybrid detected comprising said probe and a target polynucleotide comprising SEQ ID No. 1 or SEQ ID No. 5 in a control sample, wherein a reduction of the amount of detectable hybrid relative to said control hybrid is indicative of said cancerous or precancerous condition.

16. The method of claim 14 or 15, wherein said probe is covalently coupled with a detectable label.

17. The method of claim 16, wherein said detectable label is one selected from the group consisting of: radiolabel, fluorescent label, chemiluminescent label, and colorimetric label.

18. A method for diagnosis of a cancerous or precancerous condition in a mammal, said method comprising performing a detection step to detect an amplification of 50 or more consecutive nucleotide sequence of SEQ ID No. 1 or SEQ ID No. 5 in a biological sample from said mammal using one or more of primers, each said primer being complementary to said consecutive nucleotide sequence, wherein an absence of said amplification is indicative of said cancerous or precancerous condition.

19. The method of claim 18, further comprising the step of comparing the amount of said amplification detected in said biological sample with the amount of a control amplification detected comprising said primers and a target polynucleotide comprising SEQ ID No. 1 or SEQ ID No. 5 of a control sample, wherein a reduction of the amount of said amplification relative to said control amplification is indicative of said cancerous or precancerous condition.

20. The method of claim 18 or 19, wherein said amplification is by a polymerase chain reaction.

21. A method for diagnosis of a cancerous or precancerous condition in a mammal, said method comprising performing a detection step to detect the formation of a complex between an antibody and a polypeptide comprising SEQ ID No. 3, SEQ ID No. 4 or SEQ ID No. 6 in a biological sample from said mammal, wherein an absence of the formation of said complex is indicative of said cancerous or precancerous condition.

22. The method of claim 21, further comprising the step of comparing the amount of said complex detected in said biological sample with the amount of a control complex detected comprising said antibody and a target polypeptide comprising SEQ ID No. 3, SEQ ID No. 4 or SEQ ID No. 6 of a control sample, wherein a reduction of the amount of said complex relative to the amount of said control complex is indicative of said cancerous or precancerous condition.

23. The method of claim 21 or 22, wherein said antibody is covalently coupled with a detectable label.

24. The method of claim 23, wherein said detectable label is one selected from the group consisting of: radiolabel, fluorescent label, chemiluminescent label and colorimetric label.

25. The method of claim 14, 18 or 21, wherein said cancerous condition is selected from a solid tumor and a leukemia.

26. The method of claim 14, 18 or 21, wherein said mammal is human.

27. A method for reducing telomerase function in an eukaryotic cell comprising contacting said eukaryotic cell with a polynucleotide comprising SEQ ID No. 1, SEQ ID No. 2 or SEQ ID No. 5, and expressing said polynucleotide in said eukaryotic cell in an amount sufficient to reduce telomerase function.

28. A method for reducing telomerase function in an eukaryotic cell comprising contacting said eukaryotic cell with a polypeptide comprising SEQ ID No. 3, SEQ ID No. 4 or SEQ ID No. 6 in an amount sufficient to reduce telomerase function.

29. The method of claim 27, or 28, wherein said reduction of telomerase function is determined by measuring one or more of: a reduction in telomerase enzymatic activity, a reduction in telomere length, a reduction in cell proliferation, an induction of senescence, and an induction of crisis in said cell.

30. The method of claim 27, or 28, wherein said eukaryotic cell is a mammalian cell.

31. The method of claim 30, wherein said mammalian cell is a human cell.

32. A method for preventing or treating a cancerous condition in a mammal comprising administering a therapeutically effective amount of a polynucleotide comprising SEQ ID No. 1, SEQ ID No. 2 or SEQ ID No. 5.

33. A method for preventing or treating a cancerous condition in a mammal comprising administering a therapeutically effective amount of a polypeptide comprising SEQ ID No. 3, SEQ ID No. 4 or SEQ ID No. 6.

34. The method of claim 32 or 33, wherein said therapeutically effective administration results in a reduction in tumor size.

35. The method of claim 32 or 33, wherein said therapeutically effective administration results in a reduction in number of tumor cells.

36. The method of claim 32 or 33, wherein said mammal is a human.

37. The method of claim 32 or 33, wherein said polynucleotide or polypeptide is administered as a pharmaceutical composition further comprising a pharmaceutically acceptable carrier.

38. A method for increasing telomerase function in an eukaryotic cell comprising contacting said eukaryotic cell with a polynucleotide comprising an antisense polynucleotide complementary to the corresponding mRNA sequence comprising SEQ ID No. 1 or SEQ ID No. 5 in an sufficient amount to increase telomerase function.

39. A method of increasing telomerase function in an eukaryotic cell comprising contacting said eukaryotic cell with an antibody in an sufficient amount to increase telomerase function, said antibody being specifically immunoreactive with a polypeptide comprising SEQ ID No. 3 or SEQ ID No. 6.

40. The method of claim 38 or 39, wherein said increase of telomerase function is determined by measuring one or more of: an increase in telomerase enzymatic activity, an increase or maintenance in telomere length, an increase in cell proliferation, a reduction of senescence and a reduction of crisis in said cell.

41. The method of claim 38 or 39, wherein said eukaryotic cell is a mammalian cell.

42. The method of claim 41, wherein said mammalian cell is a human cell.

43. A method for preventing aging in a mammal comprising administering a therapeutically effective amount of an antisense polynucleotide complementary to the corresponding mRNA sequence comprising SEQ ID No. 1 or SEQ ID No. 5.

44. A method for preventing aging in a mammal comprising administering a therapeutically effective amount of an antibody, wherein said antibody is specifically immunoreactive with a polypeptide comprising SEQ ID No. 3 or SEQ ID No. 6.

45. The method of claim 43 or 44, wherein said mammal is a human.

46. The method of claim 43 or 44, wherein said antisense polynucleotide or antibody is administered as a pharmaceutical composition further comprising a pharmaceutically acceptable carrier.

47. A pharmaceutical composition comprising a therapeutically effective amount of a polynucleotide comprising SEQ ID No. 1, SEQ ID No. 2 or SEQ ID No. 5.

48. A pharmaceutical composition comprising a therapeutically effective amount of a polypeptide comprising SEQ ID No. 3, SEQ ID No. 4 or SEQ ID No. 6.

49. A pharmaceutical composition comprising a therapeutically effective amount of an antibody specifically immunoreactive with a polypeptide comprising SEQ ID No. 3 or SEQ ID No. 6.

50. A pharmaceutical composition comprising a therapeutically effective amount of an antisense oligonucleotide complementary to the corresponding mRNA sequence comprising SEQ ID No. 1 or SEQ ID No. 5.

51. The pharmaceutical composition of claim 47, 48, 49, or 50, further comprising a pharmaceutically acceptable carrier.

52. A method for screening for an agent which modulates the binding between a polypeptide (SEQ ID No. 3, SEQ ID No. 4 or SEQ ID No. 6) and a Pin 2 polypeptide, said method comprising:
(a) incubating a mixture comprising said polypeptide (SEQ ID No. 3 or SEQ ID No. 4), a Pin2 polypeptide, and a candidate agent, wherein said incubating whereby, but for the presence of said agent, allows said polypeptide (SEQ ID No. 3, SEQ ID No. 4 or SEQ ID No. 6) to bind to said Pin2 polypeptide to form a complex;
(b) detecting said complex formation in (a); and
(c) comparing said complex detected in (b) with a control comprising said polypeptide (SEQ ID No. 3, SEQ ID No. 4 or SEQ ID No. 6) and said Pin2 polypeptide in the absence of a candidate agent, wherein an absence, an increase, or a reduction of said complex detected in (b) is indicative of said candidate agent modulating the binding activity of said polypeptide (SEQ ID No. 3, SEQ ID No. 4 or SEQ ID No. 6) to said Pin2 polypeptide.

53. A method for screening for an agent which modulates the binding between a polypeptide comprising SEQ ID No. 3, SEQ ID No. 4 or SEQ ID No. 6 and a Pin 2 polypeptide in an eukaryetic cell, said method comprising:
(a) contacting said eukaryotic cell with a candidate agent, wherein said contacting whereby, but for the presence of said agent, allows said polypeptide comprising SEQ ID No. 3 SEQ ID No. 4 or SEQ ID No. 6 to bind to said Pin2 polypeptide to form a complex in said cell;
(b) detecting said complex formation in (a); and
(c) comparing said complex detected in (b) with a control cell without contacting said control cell to said candidate agent, wherein an absence, an increase, or a reduction of said complex formation in (b) is indicative of said candidate agent modulating the binding activity of said polypeptide comprising SEQ ID No. 3 or SEQ ID No. 4 or SEQ ID No. 6 to said Pin2 polypeptide.

54. A method for screening for an agent which modulates the binding between a polypeptide (SEQ ID No. 3, SEQ ID No. 4 or SEQ ID No. 6) and a telomerase polypeptide, said method comprising:
(a) incubating a mixture comprising said polypeptide (SEQ ID No. 3 or SEQ ID No. 4), a telomerase polypeptide, and a candidate agent, wherein said incubating whereby, but for the presence of said agent, allows said polypeptide (SEQ ID No. 3, SEQ ID No. 4 or SEQ ID No. 6) to bind to said telomerase polypeptide to form a complex;
(b) detecting said complex formation in (a); and
(c) comparing said complex detected in (b) with a control comprising said polypeptide (SEQ ID No. 3, SEQ ID No. 4 or SEQ ID No. 6) and said telomerase polypeptide in the absence of a candidate agent, wherein an absence, an increase, or a reduction of said complex detected in (b) is indicative of said candidate agent modulating the binding activity of said polypeptide (SEQ ID No. 3, SEQ ID No. 4 or SEQ ID No. 6) to said telomerase polypeptide.

55. A method for screening for an agent which modulates the binding between a polypeptide comprising SEQ ID No. 3, SEQ ID No. 4 or SEQ ID No. 6 and a telomerase polypeptide in an eukaryotic cell, said method comprising:
(a) contacting said eukaryotic cell with a candidate agent, wherein said contacting whereby, but for the presence of said agent, allows said polypeptide comprising SEQ ID No. 3 SEQ ID No. 4 or SEQ ID No. 6 to bind to said telomerase polypeptide to form a complex in said cell;
(b) detecting said complex formation in (a); and
(c) comparing said complex detected in (b) with a control cell without contacting said control cell to said candidate agent, wherein an absence, an increase, or a reduction of said complex formation in (b) is indicative of said candidate agent modulating the binding activity of said polypeptide comprising SEQ ID No. 3 or SEQ ID No. 4 or SEQ ID No. 6 to said telomerase polypeptide.

56. The method of claim 52, 53, 54 or 55, wherein said complex detection is through an antibody, said antibody being specifically immunoactive to a polypeptide comprising SEQ ID No. 3 or SEQ ID No. 6.

57. The method of claim 56, wherein said antibody is covalently coupled with a detectable label.

58. The method of claim 57, wherein said detectable label is one selected from the group consisting of: radiolabel, fluorescent label, chemiluminescent label, and colorimetric label.

59. A method for screening for an agent which modulates the expression of a polynucleotide comprising SEQ ID No. 1 or SEQ ID No. 5 in an eukaryotic cell, said method comprising:
(a) contacting said eukaryotic cell with a candidate agent;
(b) detecting the expression of said polynucleotide in said eukaryotic cell; and
(c) comparing the expression of said polynucleotide in (b) with a control cell without contacting said control cell to said candidate agent, wherein an increase or a decrease of the expression of said polynucleotide in (b) is indicative of said candidate agent modulating the expression of said polynucleotide.

60. The method of claim 59, wherein said expression detection is through a probe or a pair of primers, each said probe or primer having a sequence complementary to the sequence of said polynucleotide.

61. The method of claim 60, wherein said expression detection is by a polymerase chain reaction.

62. The method of claim 59, wherein said expression detection is through an antibody, said antibody being specifically immunoactive to a polypeptide comprising SEQ ID No. 3 or SEQ ID No. 6.

63. The method of claim 60 or 62, wherein said polynucleotide or said antibody is covalently coupled with a detectable label.

64. The method of claim 63, wherein said detectable label is one selected from the group consisting of: radiolabel, fluorescent label, chemiluminescent label, and colorimetric label.

65. A method for screening for an agent as a binding partner to a Pin2 polypeptide comprising SEQ ID No. 8, said method comprising:
(a) incubating a mixture comprising said Pin2 polypeptide and a candidate agent, wherein said incubating allows said Pin2 polypeptide to bind to its binding partners to form a complex; and
(b) detecting said complex formation between said Pin2 polypeptide and said candidate, wherein a presence of said complex formation is indicative of said candidate agent being a binding partner to said Pin2 polypeptide.

66. A method for treating a cancerous condition in a mammal comprising administering a therapeutically effective amount of an agent which enhances the binding between a PinX1 polypeptide comprising SEQ ID No. 3 or SEQ ID No. 4 or SEQ ID No. 6 to a Pin2 polypeptide, wherein said administration restores the binding between said PinX1 polypeptide and said Pin2 polypeptide to a normal level.

67. A method for treating a cancerous condition in a mammal comprising administering a therapeutically effective amount of an agent which increases the expression of a PinX1 polynucleotide comprising SEQ ID No. 1 or SEQ ID No. 5, wherein said administration restores the expression of said PinX1 polynucleotide to that of a normal level.

68. The method of claim 66 or 67, wherein said therapeutically effective administration results in a reduction in tumor size.

69. The method of claim 66 or 67, wherein said therapeutically effective administration results in a reduction in number of tumor cells.
